SEQUENCE LISTING (32 sequences)

SEQ ID NO: 1 - DNA, 206 bp, synthetic construct
  source 1..206: mol_type = other DNA
  misc_difference 115..120: note = a, c, t, g, unknown or other
taatacgactcactatagggataatcttgttgatgccttagcagcacgtaaatattggcgttaagattctaaaattatctccagtattaactgtgctgctgaagtagaaacaagnnnnnncctgcttttgcttagcttatcagactgatgttgactgttgaatctcatggcaacagcagtcgatgggctgagtaaaacatggatcg

SEQ ID NO: 2 - DNA, 2517 bp, synthetic construct
  source 1..2517: mol_type = other DNA
taatacgactcactatagggataatcttgttgatgccttagcagcacgtaaatattggcgttaagattctaaaattatctccagtattaactgtgctgctgaagtagaaacaaggtcgtttttaaacaattcgacactaattgatggccatccgaattcttttggtcgctgtctggctgtcagtaagtatgctagagtcccgttttcgtttcattaccaacactacgtccccttgcccaattagcacattagccttctctccttttgcaagattgctcagttcattgatgcttaatgctgggccatatctcttgtcttctttgcccaaaatgagaaatcctctcaggacagcagactccaccccagatgtgccttcatctggatcttcagtcaatgcacctgcatcctttccaagaactgtaagtcgtttggttgccttgttgtaattgaatactggagaattgcctcttaccagtatcctcaaccctgatcctctcacattcacagtcaatgaggaaaattgcatcctactctgttctggtggggcagcagcaaaggggagaagttttattatttggacagtgtcaaatgtcccaagcacatcccgcatttgctggaacagtgtccttacgaatccactgtaccggcttctggttgccttagggacaagagactgaaatggttcaaattccattttgttgtataacattgtgggatcttgtgaccattgaattttcacaatttcccagttcctgattatccattgataagtgttgactagcactgactcagggccattgatctcccacatcattgatgacgaataagttattgtcaacttctcagttccttgcgtttcactgacttcttcgggagacaatagtacgttccctctttgatctctaacccttaaaaatcggtcaatactcactaccactctctccgtgctggagtattcatctactcccattttgctgactcttatccctctcagcgacatctccgtgcttggggtcatgtcgggcagtattccgatcattcccatcacattgtcgatggattcaattccccagttctggaaaagcacttttgcatctttttggaaatgcctcaagagttggtgcatggggttcagtcgctggtttgccctattgacaaagttcagatcgcccctaactgccttgatcatgcaatcctcctgtgagaataccatggccacaattattgcctcagcaattgactgctcgtctctcccgcttactatcaactggatcaatctcctggttgcctttctgagaatagctgttgctcttctcccaaccattgtgaattcttcatacccttcatgtactcttattttcagtgtttggaggttgcccgttagcacttcttcttctttcttgactgatgatccgcttgtccttttgaaagtgaacccaccaaaactgaaagatgagctaatcctcaaccctattgctgccttgcatatgtctacggcttgttcctcagttggattctgtctaaggatgtccaccatccttactcctccaatctgtgtgctgtggcacatttccaagagagatgctaatgggtctgctgacactgctgctcttcttactatgtttctagcagcgataatcaaactttggtcaacatcatcatttctcacttctcctcctggagtgtacatctgctcccagcacgtcccttgggttaagtgcaacacttcaatataaacactgcctgttccgccggctactgggagaaaccttgttttacggaccaattctctttctagcatgtacgccaccatcaagggagcaattttacaatcctggagctcttctttcttctcttttgttattgccagctgtgactctgatgtcagtattcttgcccccacttcatttgggaaaacaacttccataatcacatcctgtgcctccttggcactgagatctgcatggccagggtttgtatcaactctcctccttattttaacttgatttctgaagtggacagggccgaaggtaccatgtttcaacctttcgaccttttcgaaataagttttatataccttagggtaatgaactgtacttgttgttgggccattcctattccaccatgttacggccagaggtgataccatcactcggtctgatccagcatcgtttgttttgctccagagggtttgtccttgttcattcctctctggaatcatgtccattattctcttgtctgctgtaattgggtatctcattgccatcatccacttcattctgagtgcggggttcttctcttgccttcctgatgtgtactttttgattatggccatatggtccacagtggtcttagtgagtatctcgcgagtgcgggactgcgacattagatctctcagttcttttattctctccatattgaatatatttgacctgcttttgcttagcttatcagactgatgttgactgttgaatctcatggcaacagcagtcgatgggctgagtaaaacatggatcg

SEQ ID NO: 3 - DNA, 2517 bp, synthetic construct
  source 1..2517: mol_type = other DNA
taatacgactcactatagggataatcttgttgatgccttagcagcacgtaaatattggcgttaagattctaaaattatctccagtattaactgtgctgctgaagtagaaacaaggcattttttcatgaaggacaagttaaattcattatttttgccgtctgagttcttcaatggtggaacagatcttcatgatctcagagaactcttctttcttgatccgtccagactcgaagtcgaccctggcatcaatccgggccctagacaccatggcctccaccatgctagaaattccaaccggtctcctatatgaactgctagggaaaaatttctcgaatagattgcagcacttctggtacatctgttcatcctcaagaattcccctttggcttgtgttgagaatagaacgattcctcttgggaatccaggaatgtgtagttgcaacggcatcatattccatgcttttggctggaccatgggctggcattaccacagcattgtttacagaatcaatctctttatgactgacaaagggattcaggggattacaaagtcttccccgataatcatcatccattagctcccattttaagcagacttcaggaatgtgaagattccgtatattgtataagtttggtcctccatctgatactaatagccctacttttgattgggtttgatcccacagcttctttaactcaaatgatcttctcgtctgaatttgtgtgtctcccctatggcacctatatgtgtatctgtagtctttgatgaacaattgaagagccatctgggccgttgcaggtccaaggtcattgtttatcatgttgttctttatcactgttactccaatactcatgtcagctgattcatttactccagacactccaaagctgggtagctccatgctaaaattagccacaaatccatagcgataaaaaaagcttgtgaattcaaatgtccctgtcttatttatataggacttctttttgctcatgttgattcccactaacttgcaggtcctgtagaatctgtccactcctgcttgtattccctcatggtttggtgcattcactatgagagcaaaatcgtcggatgattggagcccatcccaccagtatattgtcttggtgtatttcttttgtccaagattcagtatcgagactcccaagaccgtacttagcatgttgaacatgcccatcatcatcccaggactcagtgatgctgtgccatctattagaagaggccttattttctcaattttcttctttgttgattcattgaagtacttcaggtcaatgcttgctagcatttctgctggtatttgtgttcgaatcttcattcttttactctcgaacatgtaccctttccctagtcttgccattttgtttgagaacattatgggtgccatgctcaggatgtttctgaaccactcgggttgatttctggtgatatatgtaatcatcgccaggaacattcgaggattttgattttcattccacttagtgttgtccccagtgattgtgaaagaaatctctgtgtcttgtgaattagccatcatctttctcacaacatttgccagtttggccttcttttcattgccccctactgggagcccagactgttcaagcttttcgcaaatgctcctagctaaagtttcaacaaagtatacgaaacctctaatctgcatcccaggtgttgcgatagcccttctttttaacttgcctctctctgcatctttggtcatcgtatttaatgtcagtgctcttattagatagcctctcttattcagtctttgttttttcttccctattgttctttgcgtgaccatcttcttggtcatgttgtctcttactctcctttttctttgaaagtgggttgttatctctatttcctctttgttcattgattccattacatcctttaagaaatctattagccttcctgactcattagctgttaggccattcgatctaaagacttctatggtgttggccaatgcagttgctgccggttgatttctgtttaatgtccaatcataagtctggcgaccttgagttagtttatctacccttgtttgttgaacaacttccattgtttcaaggcatgaattctcaaatattcctgggtgggattcttcaaggaaagccatagcctctagaacacagtctgtttgtgcatacccacttggttcattatcctcaggtagtggtccatcaatcgggttgagctggggtgcaccagtctctgtgtttgtcgtccactttcccttttctgagtattggtgtgttctgtttactgtgtccatggtgtatcctgttcctgttccatggctgtatggaggatctccagtataagggaatgtggtgcttatggcattttgcgctggaatttttaggaaaagtagagtcggattgacatccattcaaatggtttgcctgcttttgcttagcttatcagactgatgttgactgttgaatctcatggcaacagcagtcgatgggctgagtaaaacatggatcg

SEQ ID NO: 4 - DNA, 2409 bp, synthetic construct
  source 1..2409: mol_type = other DNA
taatacgactcactatagggataatcttgttgatgccttagcagcacgtaaatattggcgttaagattctaaaattatctccagtattaactgtgctgctgaagtagaaacaaggtacttttttggacagtatggatagcaaatagtagcattgccacaactacttcagtgcatgtgtgaggaaggagttgaaccaagatgcattaagcaaaacccagggatcattaatcaggcactcctcgattgcttcatatagccccccaagatcgaaggttccaggttccaggttgtccctaagtgcctgaacaatgagaagcaattttctagattcagccgaaaacccctcaagttgtggagacgcatatagactgttgaatacagattttgccagtaaggtcctgcacactttcccaatagagccttcctccactcccctgggtgattctccgattggccatgtttccgatttgttttcaaagaattccttggtcatgtctttctctttgacagaagactcggcctcaatcatgctctcaatctgctgaagagactgaagaaggcagcgcctcatttccatgccccatttcatcttgatcttggaggttccattggttctcacatataggaacatgggcctcgacacttggcctatcgcagtcctcaagagcatgtctcctatttcaagaacacagtatttttcccatttgtgtggctccagtctcgggtcagtgagtgagaactccatacttacaaagttcaccacatcagtatcatttctcaaatgagaccttccttttataatgaacccatacaggtttgttttccgtcttccttctttggtcctacatttgcttatcattgggatcagctgaaagtcatccatggctgcacaggatgcattgagcaaggccgtatttatgtacactcccttcattatgtattcagtagccctgcagtgggacacttctgctgtaaaatagttcctcctcatgcttgcgatatgttcaatcggggcaacatcttctcctatttcatcaagttctatccagcttgaatcagtcaattcacatgccttattgaattcattttggacccagcttgctagagatctgggctctggctcatcactgtcatactgtttaaggtctccaacatctttgcagtcatcaaagtctactttttctggtgccatattttcaccgagtgcccacttcaattggcttgttctcttcatgttctttgtccttgggatcttctcttcattttcaatgtcctgtagctctgctagcacctgcttccaagccatgaggtaattgggatttatgcctttctcatgtggtttgactatgttaggctctttccagccaaagaatgtcttcatgcatttgattgcatcatatagtggtattccctccccctcgtgactcgggtcttcaatacttaatttcagagcatccatcagcaggaactttgaccgctgatggcaaagaggcccatcaggcaatctgagggggcgtggtgtcgtcctcaagaatggttcaattttggcgttcacttcttttgacatttgggaaagcttgccctcaatgcagccgttcggctcgaatccatctacataggctctaaagttttcaaggctggggaagttcggtgggagactttggtcggcaagcttgcgcatagttcctgtaatctcgaatttttcttcaattgtctcttcgcctctttcggactgacgaaaggaatcccatagactcctactggccatttcttgtcttatagtgaaaagcctagttttgattcttgccctgctctcttcgtcaagggtgtagtccgctttggtggccatctcctctccagtgaatgaaaagatgtgaatgtgtgtcttctcagattttattttgttggctttctctaggtaatatatgtggacttccctccgtgttactccaatttcaatgaaccggttctctttgtaatcatacaaatcaggaagaaatttaggcttctctacccctgttgtgttacatatactgttcaccactgtccaggccatgattcggtctcttccttcaattatctcaaatcggtgcttcaatagtgcattcgggtcaccagattctacaattattgattcaccccgttcgtcgatgaaatggaaatccgaatacatgaaacaaacttccaaatgtgtgcatattgcagcaaacttgttagtttcgattttcggatcttccccatattctttcattgccttttccgcaagctcgacgatcattggattgaagcattgtcgcacaaagtcttccattttggatcagtacctgcttttgcttagcttatcagactgatgttgactgttgaatctcatggcaacagcagtcgatgggctgagtaaaacatggatcg

SEQ ID NO: 5 - DNA, 1923 bp, synthetic construct
  source 1..1923: mol_type = other DNA
taatacgactcactatagggataatcttgttgatgccttagcagcacgtaaatattggcgttaagattctaaaattatctccagtattaactgtgctgctgaagtagaaacaagggtgtttttctcatgcttctgaaatcctaatgttaaatacatattctacactgtagagacccattagagcacatccagaaactgattgcccccagggagactaccagtaccaatgaactggcgacagttgaatagatcgccaaaatctggtaaatccttgttgattccagctttaccccatctatttcttctctgtttaattttgcttcctctgagtattttgggtagtcataagtcccatttttgacactttccatgcacgtgttatcgcatttgtggtaaaattcaaagcagccgtttccaatttccttggcattgttttttagctggcttcttaccttttcatataagttcttcacatttgaatcgtggtagtccaaagttctttcattttccaatagaaccaacagttcggcattgtaagtccaaatgtccaggaaaccatcatcaacttttttatttaaattctctactcttttttccaggtggttgaactctttacctactgctgtgaactgtgtattcatcttttcaataacagaatttactttgttagtaatctcgtcaatggcattctgtgtgctcttcaggtcggctgcatatcctgacccctgctcattttgatggtgataaccgtaccatccatctaccatccctgtccaccccccttcaatgaaaccggcaatggccccaaataggcctctagattgaatagacgggatattcctcaatcctgtggccagtctcaattttgtgctttttacatattttggacattttccaattgtgatcggatgtatattctgaaatgggaggctggtgtttatagcacccttgggtgtttgacaagttgtattgcaatcgtggactggtgtatctgaaatgataataccagatccagcatttctttccattgcgaatgcatatctcggtaccactagatttccagttgcttcgaatgttattttgtctcccggctctactagtgtccagtaatagttcattctcccttcttgatccctcactttgggtcttattgctatttccggcttgaacttcttgctgtatcttgatgaccccacaaaaacatatgtatctgcattctgatagagactttgttggtcagcactagtagatggatggtgaatgccccatagcacgaggacttctttccctttatcattaatgtaggatttgctgagctttgggtatgaatttccttttttaactagccatattaaatttttgtagaagctttttgctccagcatgaggacatgctgccgttacacctttgttcgagtcatgattgggccatgaacttgtcttggggaatatctcaaacctttcaaatgatgacactgagctcaattgctctcttagctcctcataatcgatgaaatctcctgggtaacacgttccattgtctgaactaggtgtttccacaatgtaggaccatgagcttgctgtggagagtgattcacactctggatttcccaggatccagccagcaatgttacatttacccaaatgcaatggggctacccctcttagtttgcatagtttcccgttatgcttgtcttctagaaggttaacagagtgtgttactgttacattcttttctagtactgtgtctacagtgtctgttgaattgttcgcatgataacctatacataatgtgtctgcatttgcggttgcaaatgtatatagcagaactactagtacctgcttttgcttagcttatcagactgatgttgactgttgaatctcatggcaacagcagtcgatgggctgagtaaaacatggatcg

SEQ ID NO: 6 - DNA, 1741 bp, synthetic construct
  source 1..1741: mol_type = other DNA
taatacgactcactatagggataatcttgttgatgccttagcagcacgtaaatattggcgttaagattctaaaattatctccagtattaactgtgctgctgaagtagaaacaagggtatttttcctcaactgtcatactcctctgcattgtctccgaagaaataagacccttcattactcatgtcaaaggaaggcacgatcgggttcgttgccttttcgtccgagagctcgaagactccccgcccctggaaggacaaatcttctggctttgcactttccatcattcttataacttctgttcgcatgtcggatgtccgtccttcattgttcccgctgaatgctgccataacggttgctctttcaaaagggagattccgctgcactgagaatgtaggctgcacactgatctggcctgcggatgccttttgttgattggtatttcctccactcctggtccttatggcccagtatctgcttctcagttccagggtattggagttcatggtttccacattctcatttgaagcaatctggacccctcttgtggaaagctttcctcttggaatcactttctttcctcttatgaaacttgatactcttaaatcttcaaatgcagcagagtggcatgccatccacaccaattgactcttgtgagctgggttttcatttggtctcatcaggctgaccacttggctgttttggagtaatttgaatgggtctatcccgaccagtgagtacccttccctttcaaagtcatgcccacttgctactgcaagcccatacacacaagcaggcaggcaggatttatgtgcaactgatcccctcagaatgagtgctgaccgtgccaggaaaatgaggtcttcaatctcagcgtttcctgggtttcgactttctcttacttgatccatcattgccctctgggcagctgtttgaaattttcctttgaggatattgcacattctttcataagcaacccttgtccttcgtccattttcacccctccagaaatttcggtcattgattccacgtttgatcattctgattaactccattgctattgttccaactcctttcaccgcagcacctgcggcaccagaccttctgggaagtgttgaaccttgcattagagagcacattctgggatccattccggtgcgaacaagcgctcttgttctctgatatgtggcatcattcaggttggaatgccaaatcatgatatgagtaagacctgctgttgcatcttcgccattgtttgcttggcgccaaactctccttatttcttctttgtcataaaggatgagttctctcatccactttccgtctactcttctatatatgggtcctcctgttttcttagggtccttcccagcactgggatgctcttctaggtatttatttcttctctcatcaaaagcagaaagcaccatcctctctattgttatgctattctggattagtcgtccatcataatcactgagtttgagttcagtgcacatttggatgtagaatctcccgattccaccaatcattcttccgacagatgctctgatttctgtggcatcctggcgctccccaccagtctccatttgttcatatgatcgtttggtgccttgagacgccatggcttcgatgtcactcattgagtgattatctaccctgcttttgcttagcttatcagactgatgttgactgttgaatctcatggcaacagcagtcgatgggctgagtaaaacatggatcg

SEQ ID NO: 7 - DNA, 1646 bp, synthetic construct
  source 1..1646: mol_type = other DNA
taatacgactcactatagggataatcttgttgatgccttagcagcacgtaaatattggcgttaagattctaaaattatctccagtattaactgtgctgctgaagtagaaacaaggagttttttgaacaaattacttgtcaatggtaaatggcaactcagcaccgtctggccaagaccaacccacagtgtcactgtttacaccacaaaaggatatgctgctcccgctagtccagattgtgttctctttgggtcgccctctgattagttcaacccagaagcaaggtcttatacaatccagccctgttagttctggatgctgaacaaaactcccgctatatcctgaccactcatttattcctacgatatcttgctttattgagaagttattgtctgtcccagtccatccgttcggatcccaaatcatctcaaaaccgtttcttgaactaatgcttttagttctccctatccaaacaccattgccgtatttgaatgaaaacccttttactccatttgctccattagacgatactggaccacaactgcctgtcttatcattagggcgtggattgtctccgaaaatcccactgcatatgtatcctatctgatattccagattctggttgaaagacacccacggtcgattcgagccatgccagttatccctgcacacacatgtgatttcactagaatcaggataacaggagcattcctcatagtgataattaggggcattcatttcgactgatttgactatctttcccttttctattctgaagatcttgtatgaggcctgtccattacttggtccatcggtcattacagtaaagcaagaaccatttacacatgcacattcagactcttgtgttctcaatatattgtttctccaactcttgatagtgtctgttattatgccgttgtactttaacacagccactgccccattgtctgggccagaaattccaattgttagccaattgatgccatcatgacaagcacttgctgaccaagcgactgactcaaatcttgagttgtatggagagggaacttcaccaataggacagctcattagggttcgatatgggctcctgtctttaatggttccattggaatgtttgtcatttagcaaggccccttgagtcaagaagaaggttctgcattccaagggggagcatgatatgaatggttcccttatgacaaacacatcccccttggaaccgattcttacactgttgtctttactgtatatagcccatccactaacagggcagagagaggaattgcccgctaatttcacggaaaccactgactgtccagcagcaaagttggtgttgctgatgttaacatatgtctgatttacccaagtgttgttttcataagtaatgacgctttgattgcatgtttcaatctgattttgattcccaagttgaattgagtggctaatccatattgagattatgtttccaatttgtaatattaagttagccattccaattgtcatacagaccgaaccaatggttattatcttttggtttggattcattttaaacccctgcttttgctcctgcttttgcttagcttatcagactgatgttgactgttgaatctcatggcaacagcagtcgatgggctgagtaaaacatggatcg

SEQ ID NO: 8 - DNA, 1203 bp, synthetic construct
  source 1..1203: mol_type = other DNA
taatacgactcactatagggataatcttgttgatgccttagcagcacgtaaatattggcgttaagattctaaaattatctccagtattaactgtgctgctgaagtagaaacaaggtagttttttactctagctctatgttgacaaaatgaccatcgtcaacatccacagcactctgctgttcctgttgatattcttccctcatggactcaggcactccttccgtagaaggccctcttttcaaaccgtatttaaagcgacgataaatacatttgaaaaaaagacgatcagtaatccacaatatcaggtgcaagatcccaatgatatttgctgcaatgacgagaggatcacttgaatcgctgcatctgcactcccattcgcttctggtaggcctgcaaattttcaagaaggtcatctttcagaccagcactggagctaggatgagtcccaatagttctcattgcatgtaccatctgcctagtctgattagcaacctccatggcctccgctgcctgttcactcgatccagccatctgttccatagcctttgccgtagtgctagccagcaccattctgttttcatgcctgattagtggattggtggtagtagccatctgtctgtgagaccgatgctgtgaatcagcaatctgttcacaagtggcacacactagaccaaaagcagcttctgtggtcactgttcccatcctgttgtatatgaggcccatgcaactggcaagtgcaccagttgaatagcttagtgacacctccttggccccatggaacgttatttctcttttgagcttcttgtatagtttaactgctctatccatgttgttcgggtccccattcccatttagggcattttggacaaagcgtctacgctgcagtcctcgctcactgggcacggtgagcgtgaacacaaatcctaaaattcccttagtcagaggtgacaagattggtcttgtctttagccattccatgagagcctcaagatctgtgttctttcctgcaaagacactttccagtttctgcgcgatctcggctttgagggggcctgacgggatgatagaaagaacgtacgtttcgacctcggttagaagactcatctttaaatatctacctgcttttgcttagcttatcagactgatgttgactgttgaatctcatggcaacagcagtcgatgggctgagtaaaacatggatcg

SEQ ID NO: 9 - DNA, 1066 bp, synthetic construct
  source 1..1066: mol_type = other DNA
taatacgactcactatagggataatcttgttgatgccttagcagcacgtaaatattggcgttaagattctaaaattatctccagtattaactgtgctgctgaagtagaaacaagggtgttttttatcattaaataagctgaaacgagaaagctcttatctcttgttctacttcaagcagtagttgtaaggcttgcataaatgttatttgttcgaaactattctctgtcgctttcaatctgtgccgcatttcttcaattaaccaccttatttcctcaaatttctgtcccaattgctctcgccacttttcatttctgctctggaggtagtgaaggtctcccattctcatcacagtttctccaagcgaatctctgtatattttcagagactcgaaccgtgttaccattccattcaagtcctccgatgaggaccccaactgcatttttgacatcctcataagtatgtcctggaagagaaggtaatggtgaaatttctccaactattgctccctcctcagtgaaagcccttagtagtatcaaggtctctaatcggttaaagattacactgaagttcgctttcagtactatgttcttttccatgatcgcctggtccaatcgcacgcaaagagggcctattatcttttgcctaggcatgagcatgaaccagtctcgtgacatttcatcgagggtcatgtcagaaaggtaccgcgaagtaggtacagatgcaattgtcattctaagtgtctcgctggattcctctttcaagatccattccacgatttgtttcccaacaagagtggctgtttcgatatcgaggccaagggtgttgcctcttccttttaaggacttttgatctcggcggagccgatcaaggaatggggcatcacccaatccattgtctgcaaatcgcttgcggatatgccaaaggaaacagtctacctgaaagcttgacatggtgttggagtccattatgtttttgtcaccctgcttttgcttagcttatcagactgatgttgactgttgaatctcatggcaacagcagtcgatgggctgagtaaaacatggatcg

SEQ ID NO: 10 - DNA, 2312 bp, synthetic construct
  source 1..2312: mol_type = other DNA
taatacgactcactatagggataataccatggaacgcattaaagaactgcgcgatctgatgagccagagccgcacccgcgaaattctgaccaaaaccaccgtggatcatatggcgattattaaaaaatataccagcggccgccaggaaaaaaacccggcgctgcgcatgaaatggatgatggcgatgcgctatccgattaccgcggataaacgcattatggatatgattccggaacgcaacgaacagggccagaccctgtggagcaaaaccaacgatgcgggcagcgatcgcgtgatggtgagcccgctggcggtgacctggtggaaccgcaacggcccgaccaccagcaccgtgcattatccgaaagtgtataaaacctattttgaaaaagtggaacgcctgaaacatggcacctttggcccggtgcattttcgcaaccaggtgaaaattcgccgccgcgtggataccaacccgggccatgcggatctgagcgcgaaagaagcgcaggatgtgattatggaagtggtgtttccgaacgaagtgggcgcgcgcattctgaccagcgaaagccagctggcgattaccaaagaaaaaaaagaagaactgcaggattgcaaaattgcgccgctgatggtggcgtatatgctggaacgcgaactggtgcgcaaaacccgctttctgccggtggcgggcggcaccggcagcgtgtatattgaagtgctgcatctgacccagggcacctgctgggaacagatgtataccccgggcggcgaagtgcgcaacgatgatgtggatcagagcctgattattgcggcgcgcaacattgtgcgccgcgcggcggtgagcgcggatccgctggcgagcctgctggaaatgtgccatagcacccagattggcggcgtgcgcatggtggatattctgcgccagaacccgaccgaagaacaggcggtggatatttgcaaagcggcgattggcctgcgcattagcagcagctttagctttggcggctttacctttaaacgcaccagcggcagcagcgtgaaaaaagaagaagaagtgctgaccggcaacctgcagaccctgaaaattcgcgtgcatgaaggctatgaagaatttaccatggtgggccgccgcgcgaccgcgattctgcgcaaagcgacccgccgcctgattcagctgattgtgagcggccgcgatgaacagagcattgcggaagcgattattgtggcgatggtgtttagccaggaagattgcatgattaaagcggtgcgcggcgatctgaactttgtgaaccgcgcgaaccagcgcctgaacccgatgcatcagctgctgcgccattttcagaaagatgcgaaagtgctgtttcagaactggggcattgaaagcattgataacgtgatgggcatgattggcattctgccggatatgaccccgagcaccgaaatgagcctgcgcggcattcgcgtgagcaaaatgggcgtggatgaatatagcagcaccgaacgcgtggtggtgagcattgatcgctttctgcgcgtgcgcgatcagcgcggcaacgtgctgctgagcccggaagaagtgagcgaaacccagggcaccgaaaaactgaccattacctatagcagcagcatgatgtgggaaattaacggcccggaaagcgtgctggtgaacacctatcagtggattattcgcaactgggaaattgtgaaaattcagtggagccaggatccgaccatgctgtataacaaaatggaatttgaaccgtttcagagcctggtgccgaaagcgacccgcagccgctatagcggctttgtgcgcaccctgtttcagcagatgcgcgatgtgctgggcacctttgataccgtgcagattattaaactgctgccgtttgcggcggcgccgccggaacagagccgcatgcagtttagcagcctgaccgtgaacgtgcgcggcagcggcctgcgcattctggtgcgcggcaacagcccggtgtttaactataacaaagcgaccaaacgcctgaccgtgctgggcaaagatgcgggcgcgctgaccgaagatccggatgaaggcaccagcggcgtggaaagcgcggtgctgcgcggctttctgattctgggcaaagaagataaacgctatggcccggcgctgagcattaacgaactgagcaacctggcgaaaggcgaaaaagcgaacgtgctgattggccagggcgatgtggtgctggtgatgaaacgcaaacgcgatagcagcattctgaccgatagccagaccgcgaccaaacgcattcgcatggcgattaactagatcg

SEQ ID NO: 11 - DNA, 2306 bp, synthetic construct
  source 1..2306: mol_type = other DNA
taatacgactcactatagggataataccatggatgtgaacccgaccctgctgtttctgaaaattccggcgcagaacgcgattagcaccacctttccgtataccggcgatccgccgtatagccatggcaccggcaccggctataccatggataccgtgaaccgcacccatcagtatagcgaaaaaggcaaatggaccaccaacaccgaaaccggcgcgccgcagctgaacccgattgatggcccgctgccggaagataacgaaccgagcggctatgcgcagaccgattgcgtgctggaagcgatggcgtttctggaagaaagccatccgggcatttttgaaaacagctgcctggaaaccatggaagtggtgcagcagacccgcgtggataaactgacccagggccgccagacctatgattggaccctgaaccgcaaccagccggcggcgaccgcgctggcgaacaccattgaagtgtttcgcagcaacggcctgaccgcgaacgaaagcggccgcctgattgattttctgaaagatgtgatggaaagcatgaacaaagaagaaattgaaattaccacccattttcagcgcaaacgccgcgtgcgcgataacatgaccaaaaaaatggtgacccagcgcaccattggcaaaaaaaaacagcgcctgaacaaacgcggctatctgattcgcgcgctgaccctgaacaccatgaccaaagatgcggaacgcggcaaactgaaacgccgcgcgattgcgaccccgggcatgcagattcgcggctttgtgtattttgtggaaaccctggcgcgcagcatttgcgaaaaactggaacagagcggcctgccggtgggcggcaacgaaaaaaaagcgaaactggcgaacgtggtgcgcaaaatgatggcgaacagccaggataccgaaattagctttaccattaccggcgataacaccaaatggaacgaaaaccagaacccgcgcatgtttctggcgatgattacctatattacccgcaaccagccggaatggtttcgcaacattctgagcatggcgccgattatgtttagcaacaaaatggcgcgcctgggcaaaggctatatgtttgaaagcaaacgcatgaaaattcgcacccagattccggcggaaatgctggcgagcattgatctgaaatattttaacgaaagcaccaaaaaaaaaattgaaaaaattcgcccgctgctgattgatggcaccgcgagcctgagcccgggcatgatgatgggcatgtttaacatgctgagcaccgtgctgggcgtgagcattctgaacctgggccagaaaaaatataccaaaaccatttattggtgggatggcctgcagagcagcgatgattttgcgctgattgtgaacgcgccgaaccatgaaggcattcaggcgggcgtggatcgcttttatcgcacctgcaaactggtgggcattaacatgagcaaaaaaaaaagctatattaacaaaaccggcacctttgaatttaccagctttttttatcgctatggctttgtggcgaactttagcatggaactgccgagctttggcgtgagcggcgtgaacgaaagcgcggatatgagcattggcgtgaccgtgattaaaaacaacatgattaacaacgatctgggcccggcgaccgcgcagatggcgctgcagctgtttattaaagattatcgctatacctatcgctgccatcgcggcgatacccagattcagacccgccgcagctttgaactgaaaaaactgtgggatcagacccagagcaaagtgggcctgctggtgagcgatggcggcccgaacctgtataacattcgcaacctgcatattccggaagtgtgcctgaaatgggaactgatggatgatgattatcgcggccgcctgtgcaacccgctgaacccgtttgtgagccataaagaaattgatagcgtgaacaacgcggtggtgatgccggcgcatggcccggcgaaaagcatggaatatgatgcggtggcgaccacccatagctggattccgaaacgcaaccgcagcattctgaacaccagccagcgcggcattctggaagatgaacagatgtatcagaaatgctgcaacctgtttgaaaaattttttccgagcagcagctatcgccgcccggtgggcattagcagcatggtggaagcgatggtgagccgcgcgcgcattgatgcgcgcgtggattttgaaagcggccgcattaaaaaagaagaatttagcgaaattatgaaaatttgcagcaccattgaagaactgcgccgccagaaatagatcg

SEQ ID NO: 12 - DNA, 2183 bp, synthetic construct
  source 1..2183: mol_type = other DNA
taatacgactcactatagggataataccatggaagattttgtgcgccagtgctttaacccgatgattgtggaactggcggaaaaagcgatgaaagaatatggcgaagatccgaaaattgaaaccaacaaatttgcggcgatttgcacccatctggaagtgtgctttatgtatagcgattttcattttattgatgaacgcggcgaaagcattattgtggaaagcggcgatccgaacgcgctgctgaaacatcgctttgaaattattgaaggccgcgatcgcattatggcgtggaccgtggtgaacagcatttgcaacaccaccggcgtggaaaaaccgaaatttctgccggatctgtatgattataaagaaaaccgctttattgaaattggcgtgacccgccgcgaagtgcatatttattatctggaaaaagcgaacaaaattaaaagcgaaaaaacccatattcatatttttagctttaccggcgaagaaatggcgaccaaagcggattataccctggatgaagaaagccgcgcgcgcattaaaacccgcctgtttaccattcgccaggaaatggcgagccgcagcctgtgggatagctttcgccagagcgaacgcggcgaagaaaccattgaagaaaaatttgaaattaccggcaccatgcgcaaactggcggatcagagcctgccgccgaactttccgagcctggaaaactttcgcgcgtatgtggatggctttgaaccgaacggctgcattgaaggcaaactgagccagatgagcaaagaagtgaacgcgaaaattgaaccgtttctgcgcaccaccccgcgcccgctgcgcctgccggatggcccgctgtgccatcagcgcagcaaatttctgctgatggatgcgctgaaactgagcattgaagatccgagccatgaaggcgaaggcattccgctgtatgatgcgattaaatgcatgaaaaccttttttggctggaaagaaccgaacattgtgaaaccgcatgaaaaaggcattaacccgaactatctgatggcgtggaaacaggtgctggcggaactgcaggatattgaaaacgaagaaaaaattccgcgcaccaaaaacatgaaacgcaccagccagctgaaatgggcgctgggcgaaaacatggcgccggaaaaagtggattttgatgattgcaaagatgtgggcgatctgaaacagtatgatagcgatgaaccggaaccgcgcagcctggcgagctgggtgcagaacgaatttaacaaagcgtgcgaactgaccgatagcagctggattgaactggatgaaattggcgaagatgtggcgccgattgaacatattgcgagcatgcgccgcaactattttaccgcggaagtgagccattgccgcgcgaccgaatatattatgaaaggcgtgtatattaacaccgcgctgctgaacgcgagctgcgcggcgatggatgattttcagctgattccgatgattagcaaatgccgcaccaaagaaggccgccgcaaaaccaacctgtatggctttattattaaaggccgcagccatctgcgcaacgataccgatgtggtgaactttgtgagcatggaatttagcctgaccgatccgcgcctggaaccgcataaatgggaaaaatattgcgtgctggaaattggcgatatgctgctgcgcaccgcgattggccaggtgagccgcccgatgtttctgtatgtgcgcaccaacggcaccagcaaaattaaaatgaaatggggcatggaaatgcgccgctgcctgctgcagagcctgcagcagattgaaagcatgattgaagcggaaagcagcgtgaaagaaaaagatatgaccaaagaattttttgaaaacaaaagcgaaacctggccgattggcgaaagcccgcgcggcgtggaagaaggcagcattggcaaagtgtgccgcaccctgctggcgaaaagcgtgtttaacagcctgtatgcgagcccgcagctggaaggctttagcgcggaaagccgcaaactgctgctgattgtgcaggcgctgcgcgataacctggaaccgggcacctttgatctgggcggcctgtatgaagcgattgaagaatgcctgattaacgatccgtgggtgctgctgaacgcgagctggtttaacagctttctgacccatgcgctgaaatagatcg

SEQ ID NO: 13 - DNA, 1610 bp, synthetic construct
  source 1..1610: mol_type = other DNA
taatacgactcactatagggataataccatgcgctttaacagccagcatcagagcgataaactgagcaaaagccgcgtggataaccatagcatgagcgatattgaagcgatggcgagccagggcaccaaacgcagctatgaacagatggaaaccggcggcgaacgccaggatgcgaccgaaattcgcgcgagcgtgggccgcatgattggcggcattggccgcttttatattcagatgtgcaccgaactgaaactgagcgattatgatggccgcctgattcagaacagcattaccattgaacgcatggtgctgagcgcgtttgatgaacgccgcaacaaatatctggaagaacatccgagcgcgggcaaagatccgaaaaaaaccggcggcccgatttatcgccgcgtggatggcaaatggatgcgcgaactgattctgtatgataaagaagaaattcgccgcgtgtggcgccaggcgaacaacggcgaagatgcgaccgcgggcctgacccatattatgatttggcatagcaacctgaacgatgcgacctatcagcgcacccgcgcgctggtgcgcaccggcatggatccgcgcatgtgcagcctgatgcagggcagcaccctgccgcgccgcagcggcgcggcgggcgcggcggtgaaaggcgtgggcaccattgcgatggaactgattcgcatgattaaacgcggcattaacgatcgcaacttttggcgcggcgaaaacggccgccgcacccgcgtggcgtatgaacgcatgtgcaacattctgaaaggcaaatttcagaccgcggcgcagcgcgcgatgatggatcaggtgcgcgaaagccgcaacccgggcaacgcggaaattgaagatctgatttttctggcgcgcagcgcgctgattctgcgcggcagcgtggcgcataaaagctgcctgccggcgtgcgtgtatggcctggcggtggcgagcggccatgattttgaacgcgaaggctatagcctggtgggcattgatccgtttaaactgctgcagaacagccaggtggtgagcctgatgcgcccgaacgaaaacccggcgcataaaagccagctggtgtggatggcgtgccatagcgcggcgtttgaagatctgcgcgtgagcagctttattcgcggcaaaaaagtgattccgcgcggcaaactgagcacccgcggcgtgcagattgcgagcaacgaaaacgtggaaaccatgaacagcaacaccctggaactgcgcagccgctattgggcgattcgcacccgcagcggcggcaacaccaaccagcagaaagcgagcgcgggccagattagcgtgcagccgacctttagcgtgcagcgcaacctgccgtttgaacgcgcgaccgtgatggcggcgtttagcggcaacaacgaaggccgcaccagcgatatgcgcaccgaagtgattcgcatgatggaaagcgcgaaaccggaagatctgagctttcagggccgcggcgtgtttgaactgagcgatgaaaaagcgaccaacccgattgtgccgagctttgatatgagcaacgaaggcagctatttttttggcgataacgcggaagaatatgatagctagatcg

SEQ ID NO: 14 - DNA, 25 bp, synthetic construct
  source 1..25: mol_type = other DNA
taatacgactcactatagggataat

SEQ ID NO: 15 - DNA, 89 bp, synthetic construct
  source 1..89: mol_type = other DNA
cttgttgatgccttagcagcacgtaaatattggcgttaagattctaaaattatctccagtattaactgtgctgctgaagtagaaacaag

SEQ ID NO: 16 - DNA, 82 bp, synthetic construct
  source 1..82: mol_type = other DNA
cctgcttttgcttagcttatcagactgatgttgactgttgaatctcatggcaacagcagtcgatgggctgagtaaaacatgg

SEQ ID NO: 17 - RNA, 15 nt, synthetic construct
  source 1..15: mol_type = other RNA
agtagaaacaaggtg

SEQ ID NO: 18 - RNA, 14 nt, synthetic construct
  source 1..14: mol_type = other RNA
cacctgcttttgct

SEQ ID NO: 19 - RNA, 89 nt, synthetic construct
  source 1..89: mol_type = other RNA
gtcagcagtgccttagcagcacgtaaatattggcgttaagattctaaaattatctccagtattaactgtgctgctgaagtaaggttgac

SEQ ID NO: 20 - RNA, 89 nt, synthetic construct
  source 1..89: mol_type = other RNA
cttgttgatgccttagcagcacgtaaatattggcgttaagattctaaaattatctccagtattaactgtgctgctgaagtagaaacaag

SEQ ID NO: 21 - RNA, 81 nt, synthetic construct
  source 1..81: mol_type = other RNA
caccttgtcggatagcttatcagactgatgttgactgttgaatctcatggcaacacagtcgatgggctgtctgacattttg

SEQ ID NO: 22 - RNA, 81 nt, synthetic construct
  source 1..81: mol_type = other RNA
cctgcttttgcttagcttatcagactgatgttgactgttgaatctcatggcaacacagtcgatgggctgagtaaaacatgg

SEQ ID NO: 23 - RNA, 13 nt, Influenza A virus
  source 1..13: mol_type = genomic RNA
agtagaaacaagg

SEQ ID NO: 24 - RNA, 12 nt, Influenza A virus
  source 1..12: mol_type = genomic RNA
ccttgttcttct

SEQ ID NO: 25 - RNA, 14 nt, Influenza B virus
  source 1..14: mol_type = genomic RNA
agtagtaacaagag

SEQ ID NO: 26 - RNA, 18 nt, Influenza B virus
  source 1..18: mol_type = genomic RNA
tatacctctgcttctgct

SEQ ID NO: 27 - RNA, 11 nt, Influenza C virus
  source 1..11: mol_type = genomic RNA
tcgtcttcgtc

SEQ ID NO: 28 - RNA, 12 nt, Influenza C virus
  source 1..12: mol_type = genomic RNA
gaacgatgacga

SEQ ID NO: 29 - RNA, 11 nt, Influenza D virus
  source 1..11: mol_type = genomic RNA
tcgtattcgtc

SEQ ID NO: 30 - RNA, 11 nt, Influenza D virus
  source 1..11: mol_type = genomic RNA
aacgatgacga

SEQ ID NO: 31 - RNA, 17 nt, Thogotovirus thogotoense
  source 1..17: mol_type = genomic RNA
agagaaatcaaggcgct

SEQ ID NO: 32 - RNA, 16 nt, Thogotovirus thogotoense
  source 1..16: mol_type = genomic RNA
agcgcctgtttttgct

(19) United States
(12) Patent Application Publication
(10) Pub. No.: US 2024/0294882 A1
(43) Pub. Date: Sep. 5, 2024

(54) RNA-ONLY DERIVED ORTHOMYXOVIRUSES AND ASSOCIATED VECTORS AND USES THEREOF

(71) Applicant: NEW YORK UNIVERSITY, New York, NY (US)

(72) Inventor: Benjamin tenOever, New York, NY (US)

(73) Assignee: NEW YORK UNIVERSITY, New York, NY (US)

(21) Appl. No.: 18/593,452

(22) Filed: Mar. 1, 2024

Related U.S. Application Data
(60) Provisional application No. 63/449,457, filed on Mar. 2, 2023.

(51) Int. Cl. C12N 7/00; A61K 39/00; A61K 39/145; C07K 14/005; C12N 9/12; C12N 15/113

(52) U.S. Cl. CPC C12N 7/00; A61K 39/145; C07K 14/005; C12N 9/127; C12N 15/113; C12Y 207/07048; A61K 2039/5254; C12N 2310/141; C12N 2310/531; C12N 2760/16122; C12N 2760/16134; C12N 2760/16151

(57) ABSTRACT

The present application provides an RNA-based system for generating a virus of the Orthomyxoviridae family, and methods of producing viruses of the Orthomyxoviridae family using such system.

### CROSS REFERENCE TO RELATED APPLICATIONS

This application claims priority to U.S. Provisional Application No. 63/449,457, filed Mar. 2, 2023, the disclosure of which is herein incorporated by reference in its entirety.

### SEQUENCE LISTING

The instant application contains a Sequence Listing which has been submitted electronically in XML file format and is hereby incorporated by reference in its entirety. Said XML copy, created on Feb. 29, 2024, is named 243735_000332_SL.xml and is 53,150 bytes in size.

### TECHNICAL FIELD

The present disclosure, in some aspects, is directed to an RNA-based system for generating viruses belonging to the Orthomyxoviridae family. In other aspects, the present disclosure is directed to methods of production of viruses of the Orthomyxoviridae family.

### BACKGROUND

Orthomyxoviridae comprise a family of viruses that encode their genome in segments of negative-sense RNA. The RNA-dependent RNA-polymerase (RdRp) of this family comprises three open reading frames (ORFs), generally denoted as PB2, PB1, and PA encoded on segments 1-3, respectively. This RdRp complex recognizes distinct terminal ends of the cognate viral RNAs (vRNAs) which, in part, also define each genus (e.g. Alphainfluenzavirus, Betainfluenzavirus). In addition, the nucleoprotein (NP) viral gene product must be bound to vRNAs, forming viral ribonucleoprotein (vRNP) complexes, to enable successful RdRp amplification of any RNA longer than ˜76 nucleotides (Turrell et al., Nat. Comm. 4(1591): 1-11).

One of the limiting factors in producing viruses from the Orthomyxoviridae family is the need for precise 3′ and 5′ termini recognizable by RdRp on each of the vRNA segments, in combination with an available cell-based system that is amenable to plasmid transfection and a productive infection. The biology underlying these requirements stems from the need to generate a higher order vRNP structure composed of a double stranded substrate formed by the termini, which serve as a recognition and docking site for the RdRp (Tomescue et al. PNAS 111(32) E3335-42(2014)). In addition to lacking a 5′ cap, a 3′ poly A tail, the termini of the Orthomyxoviridae vRNAs must be represented by A or U bases, preventing the direct use of conventional DNA-dependent RNA polymerases used for in vitro synthesis such as T3, T7 or SP6 polymerases as these initiate with a G (Jorgensen et al. J Biol Chem. 266(1) 645-51 (1991)). To circumvent these issues, the use of DNA-dependent RNA polymerase I promoters, which can initiate with a 5′ A or U, coupled to a 3′ hepatitis delta virus ribozyme to ensure the proper terminal base, are commonly utilized (tenOever Cold Spring Harb Perspect Med. 10(11) 1-10 (2020)). While more recent work has demonstrated that members of the Paramyxoviridae family of viruses can be generated from plasmid DNA using T7 polymerase and a combination of 5′ and 3′ ribozymes, these two motifs fail to successfully generate (“rescue”) members of the Orthomyxoviridae family for reasons that remain unclear. In addition, leveraging 5′ and 3′ ribozymes demand specific concentrations of Mg²⁺ ions that are not amenable to high fidelity in vitro transcription (IVT) reactions, providing an additional impediment to viral generation. As a result, use of T7 to generate other viruses of the negarnavirus order demand T7 polymerase be expressed in the cytoplasm with the use of a Vaccinia helper virus or a stable cell line which creates issues with chemistry, manufacturing, and controls (CMC).

While existing techniques have proven to be effective in enabling plasmid-based generation of a subset of negative sense RNA viruses, the necessity for plasmid transfection to achieve this biology severely limits both the types of vRNAs that can be generated and the viruses and/or vectors that can be successfully launched. These restrictions derive from the necessity to transfect cells permissive to the virus of interest and a working knowledge of the promoter elements of that given species.

### SUMMARY OF THE INVENTION

The present invention provides a system to generate diverse members of the Orthomyxoviridae family using an RNA-based system (e.g., independent of DNA plasmids). As delivery of RNA only requires cytoplasmic entry, this methodology is not restricted to available transfectable cell lines nor knowledge of species-specific promoter requirements, as is the case for DNA-based rescue systems.

Thus, in some aspects, provided herein is an RNA-based system for generating a virus of the Orthomyxoviridae family, the system comprising: (i) one or more mRNAs encoding the cognate viral RNA-dependent RNA polymerase (RdRp) and a nucleoprotein (NP); and, (ii) a plurality of in vitro transcribed (“IVT”) viral RNA (“IVT-vRNA”) collectively comprising the genome of the virus, wherein each IVT-vRNA of the plurality of IVT-vRNAs comprise a genomic segment of the virus (vRNA), a miRNA hairpin structure at the 5′ end of the vRNA, and optionally a miRNA hairpin structure the 3′ end of the vRNA, and wherein the miRNA hairpin structure(s) in the IVT-vRNA can be cleaved in a host cell comprising a miRNA processing machinery to produce the vRNA with the termini that are recognizable by the cognate viral RdRp for transcription and replication.

In some embodiments, at least one of the IVT-vRNAs further comprises a miRNA hairpin structure at the 3′ end of the vRNA. The miRNA hairpin structure at the 3′ end of the vRNA (i) may the same as or different from the miRNA hairpin structure at the 5′ end of the vRNA, and (ii) can be cleaved in a host cell comprising a miRNA processing machinery to produce the vRNA with the termini that are recognizable by the cognate viral RdRp for transcription and replication.

In some embodiments, the at least one IVT-vRNAs which comprises a miRNA hairpin structure at the 3′ end of the vRNA further comprises a polyA sequence at the 3′ end of the miRNA hairpin structure located at the 3′ end of vRNA.

In some embodiments, the at least one IVT-vRNA further comprises a 5′ cap at the 5′ end of the miRNA hairpin structure located at the 5′ end of vRNA.

In some embodiments, the miRNA hairpin structure has a stem length of about 25 to about 45 nucleotides. In some embodiments, the miRNA hairpin structure has an apical loop size of about 3 to about 23 nucleotides. In some embodiments, the miRNA hairpin structure comprises a CNNC nucleotide motif or an UG nucleotide motif. In some embodiments, the miRNA hairpin structure is derived from a primary miRNA (pri-miRNA), such as pri-miR-16 or pri-miR-21a.

In some embodiments, the one or more mRNAs comprise a 5′ cap. In some embodiments, the one or more mRNAs comprise a 3′ polyA sequence.

In some embodiments, the one or more mRNAs are on separate nucleic acid molecules.

In some embodiments, the one or more mRNAs are provided in a single nucleic acid molecule.

In some embodiments, cleavage of the miRNA hairpin structure(s) in the plurality of IVT-vRNAs in a host cell comprising a miRNA processing machinery produces transcriptionally active vRNAs.

In some embodiments, the virus is replication competent or conditionally competent.

In some embodiments, the virus is of a genus selected from the group consisting of Alphainfluenzavirus, Betainfluenzavirus, Gammainfluenzavirus, Deltainfluenzavirus, Isavirus, Thogotovirus, and Quaranjavirus. In some embodiments, the virus is an influenza virus, for example, a Type A influenza virus (IAV), a Type B influenza virus (IBV), a Type C influenza virus (ICV), and a Type D influenza virus (IDV), or a variant, subtype, or reassortant thereof.

In some embodiments, the one or more mRNAs encode PB2, PB1, PA, and NP. In some embodiments, the plurality of IVT-vRNAs comprise: (i) IVT-vRNA 1 for PB2; (ii) IVT-vRNA 2 for PB1; (iii) IVT-vRNA 3 for PA; (iv) IVT-vRNA 4 for HA; (v) IVT-vRNA 5 for NP; (vi) IVT-vRNA 6 for NA; (vii) IVT-vRNA 7 for M1 and M2; and (viii) IVT-vRNA 8 for NS1 and NS2.

In some embodiments, one or more of the IVT-vRNAs comprise a heterologous nucleic acid sequence. In some embodiments, at least one of the IVT-vRNAs comprise a heterologous nucleic acid sequence. In some embodiments, at least two of the IVT-vRNAs comprise a heterologous nucleic acid sequence. In some embodiments, the IVT-vRNA comprising a heterologous nucleic acid sequence comprises IVT-vRNA 1, IVT-vRNA 2, IVT-vRNA 3, IVT-vRNA 4, IVT-vRNA 5, IVT-vRNA 6, IVT-vRNA 7, and/or IVT-vRNA 8. In some embodiments, the heterologous nucleic acid sequence is inserted at a 5′ non-coding region of the vRNA. In some embodiments, the heterologous nucleic acid sequence is inserted at a 3′ non-coding region of the vRNA. In some embodiments, the heterologous nucleic acid sequence comprises a complement of a coding sequence encoding a recombinant protein. In some embodiments, the heterologous nucleic acid has a length of about 800 to about 4,000 nucleotides.

In some aspects, provided herein is a method of producing a plurality of viruses of the Orthomyxoviridae family, or recombinant derivatives thereof, in a cell culture, comprising: (i) introducing the RNA-based system of any one of the preceding embodiments into a population of host cells, wherein the population of host cells is capable of supporting replication of the virus; and (ii) culturing the population of host cells; and (iii) recovering a plurality of the viruses. In some embodiments, the RNA-based system is introduced into the population of host cells via transfection. In some embodiments, the host cell is a mammalian cell, an insect cell, a plant cell, or a bird cell. In some embodiments, the virus is attenuated. In some embodiments, the method further comprises inactivating the virus.

In some aspects, provided herein is a method of treating or preventing a disease or disorder associated with an infection by a virus of the Orthomyxoviridae family, or recombinant derivatives thereof, in a subject in need thereof, comprising administering to the subject an effective amount of the virus produced using a method of any one of the preceding embodiments. In some embodiments, the virus is administered by intramuscular injection, intranasal administration, or inhalation administration. In some embodiments, the subject is a human.

In some aspects, provided herein is a DNA-based template for producing at least one of the plurality of IVT-vRNAs in the RNA-based system of any one of the preceding claims, comprising a complementary DNA sequence corresponding to the at least one IVT-vRNA. In some embodiments, the DNA-based template further comprises a promoter upstream of the complementary DNA sequence. In some embodiments, the DNA-based template further comprises a sequence encoding a ribozyme or a primary miRNA hairpin. In some embodiments, the promoter is selected from the group consisting of: T7 promoter, T3 promoter, and SP6 promoter.

In some aspects, provided herein is a method of making at least one of the plurality of IVT-vRNAs in the RNA-based system of any one of the preceding embodiments, comprising subjecting the DNA-based template of any one of the preceding embodiments to in vitro transcription, thereby obtaining the at least one IVT-vRNA.

### BRIEF DESCRIPTION OF THE DRAWINGS

The drawings illustrate certain embodiments of the features and advantages of this disclosure. These embodiments are not intended to limit the scope of the appended claims in any manner.

The patent or application file contains at least one drawing executed in color. Copies of this patent or patent application publication with color drawing(s) will be provided by the Office upon request and payment of the necessary fec.

FIGS. 1A-1B shows an exemplary schematic of an in vitro-based generation (“rescue”) of influenza A virus (IAV). The terminal ends of in vitro transcribed (IVT) viral RNAs (IVT-vRNAs) are precisely defined so the vRNA-dependent RNA polymerase (RdRp) can recognize them. They fold into a particular hairpin structure referred to as the ‘corkscrew’ structure (left panel of FIG. 1A). These precise ends cannot be made in vitro with recombinant polymerases such as T7, T4, or SP6 as these need the first base to be a G (FIG. 1B). While Pol I can start with an A (the required 5′ base of IAV vRNA), it does not terminate at a specific location and is not amenable to perform IVT. For this reason, viral generation relies on transfected plasmids to generate vRNA with Pol I to create the 5′ termini and a 3′ hammerhead ribozyme to result in sequence-specific cleavage. Figure discloses SEQ ID NOs: 17-18, respectively, in order of appearance.

FIGS. 2A-2B show an illustration of RNA-based system for generating (“rescuing”) a virus, in a lipid nanoparticle, using i) four capped and polyadenylated mRNAs encoding PB2, PB1, PA, and NP (FIG. 2A), and ii) eight vRNAs each flanked by at least a 5′ miRNA hairpin, e.g., IVT-vRNAs (FIG. 2B).

FIG. 3 shows an exemplary Influenza A virus (IAV), comprising a segmented genome (8 viral RNAs, i.e., vRNAs) in negative polarity bound by NP and the RdRp to form ribonucleoprotein complexes (8 vRNPs).

FIG. 4 shows an exemplary schematic illustrating the use of microRNA (miRNA) hairpins at the 5′ and/or 3′ end of RNA to enable precise processing of vRNA templates by IVT, with or without capping and/or polyadenylation of the IVT-vRNA.

FIGS. 5A-5B show schematics of the primary miR-16 (FIG. 5A; SEQ ID NOs: 19-20, respectively, in order of appearance) and miR-21a miRNAs (FIG. 5B; SEQ ID NOs: 21-22, respectively, in order of appearance), with the resulting duplex RNA illustrated with a dashed box. The primary miRNAs were modified to leverage the host microprocessor to liberate the desired 5′ end of IAV vRNA (miRNA-vRNA-5p, FIG. 5A; miRNA-vRNA-3p, FIG. 5B), circled.

FIG. 6A shows an exemplary northern blot showing total RNA from wild type or RNaseIII^{-/-} cells transfected with P32-end-labeled miRNA-vRNA-5p or miRNA-vRNA-3p constructs. Bands denote the unprocessed full-length IVT RNA (top panel) and the 5′ termini liberated from the IVT product following RNascIII processing.

FIG. 6B shows titers from allantois fluid of embryonated eggs transfected with the IVT products set forth in SEQ ID NOs: 2-13, representing precursor RNAs for IVT-vRNA segments 1-8 and mRNA for PB2, PB1, PA, and NP. Conditions included transfection of products generated following no treatment (No IVT) or IVT.

FIG. 7A shows the design of 8 influenza A virus (IAV) IVT-vRNA segments. As shown, IVT-vRNA 8 for NS1 and NS2 has been incorporated into IVT-vRNA 1 for PB2 and IVT-vRNA 2 for PB1, respectively. IVT-vRNA 8 now has the ORF for GFP.

FIG. 7B shows images of cells transfected with IAV-GFP using the RNA-based system provided herein express GFP from a heterologous nucleic acid sequence on an IVT-vRNA.

### DETAILED DESCRIPTION

In some aspects, provided herein is an RNA-based system for generating a virus of the Orthomyxoviridae family, or a recombinant derivative thereof. In some embodiments, the RNA-based system comprises (i) one or more mRNAs encoding an RNA-dependent RNA polymerase (RdRp) and a nucleoprotein of the virus; and, (ii) a plurality of in vitro transcribed (“IVT”) viral RNA (“IVT-vRNA”) collectively comprising the genome of the virus, wherein each IVT-vRNA of the plurality of IVT-vRNAs comprise a genomic segment of the virus (vRNA), wherein each IVT-vRNA is modified to comprise a miRNA hairpin structure at the 5′ end, and optionally the 3′ end, of the vRNA, and wherein the miRNA hairpin structure(s) in the IVT-vRNA can be cleaved in a host cell comprising a miRNA processing machinery to produce the vRNA with the termini that are recognizable by the cognate viral RdRp for transcription and replication.

The present disclosure is based, in part, on the inventors' surprising finding that primary microRNA (pri-miRNA) processing, which canonically only occurs in the nucleus, can be leveraged to generate the required 3′ and 5′ terminal ends of a virus of the Orthomyxoviridae family, to enable virus generation directly following the introduction of IVT-vRNA. The RNA-based systems provided herein may enable the generation of a virus of the Orthomyxoviridae family, as well as the therapeutic delivery for an array of biologic cargoes.

To generate a virus from nucleic acid (e.g., plasmid DNA), the minimal requirements are a functional RNA-dependent RNA polymerase (RdRp) and nucleoprotein (NP) for binding and amplifying viral RNAs (vRNAs), and 8 vRNA segments encoding the segmented, negative-strand viral genome. Traditionally, this was performed with 12 DNA plasmids: 4 plasmids under the control of a host DNA-dependent RNA-polymerase II (Pol II) which generates capped and poly-adenylated mRNAs that can be translated to make the core components of the RdRp (i.e., PB2, PB1, and PA), and NP; and, 8 plasmids controlled by the host DNA-dependent RNA-polymerase I (Pol I) which generates unmodified vRNA segments.

Generating a virus using plasmid DNA demands transfection into cells which take up foreign DNA readily. The DNA plasmids are transcribed by the transcriptional machinery of the host cell in the nucleus (e.g., Pol I and Pol II) to generate the 8 vRNAs and mRNA for the three RdRp subunits (e.g., PB2, PB1, PA) and NP (which is needed to structurally support the vRNAs). Following transfection, the cells and/or supernatant is used to further inoculate fertilized chicken eggs or highly susceptible cells (e.g. MDCK cells) to amplify the resulting virus.

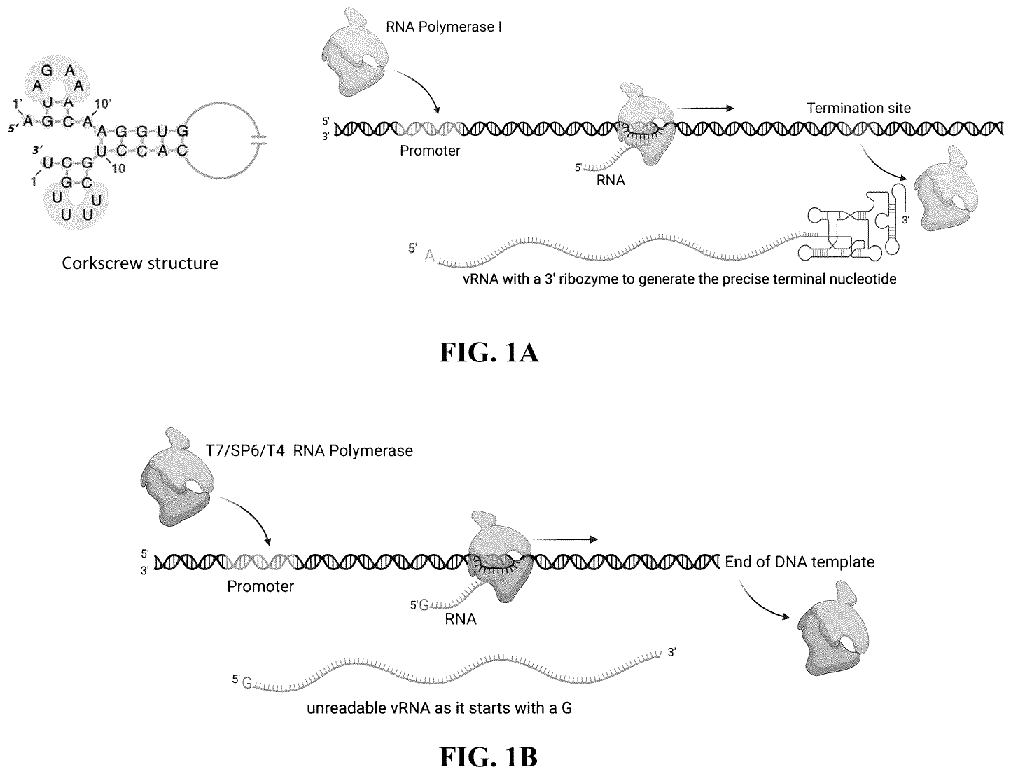

As shown in FIG. 1A, the success of DNA-based generation of a virus of the Orthomyxoviridae family demands that the terminal ends of the vRNA be precisely defined, so the RdRp can recognize them for transcription and replication. More specifically, the ends of the vRNAs fold into a particular hairpin structure historically referred to as the ‘panhandle’ or ‘corkscrew structure’ (left panel, FIG. 1A). Non-limiting examples of terminal sequences of vRNAs from a virus of the Orthomyxoviridae family are provided in Table 1 below.

TABLE 1

| | | SEQ ID | |
|---|---|---|---|
| Virus | Terminal sequence | NO | Citation |
| Influenza | 3′ end: 5′-AGUAGAAACAAGG-3′ | 23 | Flick et al. RNA |
| A virus | 5′ end: 3′-UCUUCUUGUUCC-5′ | 24 | (1996), 2:1046-1057. |
| Influenza | 3′ end: 5′-AGUAGUAACAAGAG-3′ | 25 | Reich et al. Nature |
| B virus | 5′ end: 3′-UCGUCUUCGUCUCCAUAU-5′ | 26 | (2014), 516:361-366 |
| Influenza | 3′ end: 5′-UCGUCUUCGUC-3′ | 27 | Crescenzo-Chaigne et |
| C virus | 5′ end: 3′-AGCAGUAGCAAG-5′ | 28 | al. Virology Journal |
| | | | (2008), 132: |
| | | | doi.org/10.1186/1743- |
| | | | 422X-5-132 |
| Influenza | 3′ end: 5′-UCGUAUUCGUC-3′ | 29 | Yu et al. Journal of |
| D virus | 5′ end: 3′-AGCAGUAGCAA-5′ | 30 | Virology (2019), |
| | | | 93:10.1128/jvi.01186-19. |
| Thogoto | 3′ end: 5′-AGAGAAAUCAAGGCGCU-3′ | 31 | Leahy et al. Journal of |
| virus | 5′ end: 3′-UCGUUUUUGUCCGCGA-5′ | 32 | Virology (1997), |
| | | | 71:8352-8356 |
| Infectious | 3′ end: 5′-UCGAUUCU-3′ | — | Cardenas et al. J. Fish |
| salmon | 5′ end: 3′-AGUAAAAA-5′ | — | Dis. (2020) |
| anaemia | | | 43(12): 1483-1496 |
| virus | | | |

The precise ends of the vRNAs cannot be made with recombinant polymerases such as T7, T4, or SP6 polymerases as these need the first base to be a ‘G’ nucleotide. While Pol I can start with an ‘A’ nucleotide (e.g., the required 5′ base of influenza type A (IAV) vRNA), Pol I does not terminate at a specific location. For this reason, virus generation relies both on utilization of Pol I and on a 3′ hammerhead ribozyme to result in sequence specific cleavage.

The need to use plasmid DNA to generate a virus limits the types of viruses that can be generated, as there are very few cell lines that are as transfectable as select mammalian cells (e.g., 293T, BHK21, Cos7 cells). As a result, the generation of non-mammalian members from the Orthomyxoviridae family can be very challenging. This issue could be circumvented if RNA can be used directly, since introducing foreign RNA into cells is significantly easier to achieve. Moreover, use of RNA is more compatible with any type of cell, regardless of whether it comprises human biological machinery (e.g., species-compatible promoters). However, an additional problem arises with an RNA-only derived vector methodology, because the 5′ terminal end of the vRNA segments will always contain a ‘G’ nucleotide given the availability of DNA-dependent RNA polymerases amenable to IVT reactions (FIG. 1B). While 5′ and 3′ ribozymes could also be used to solve this issue, recombinant polymerases (e.g., T7, T4, or SP6 RNA polymerases) show low fidelity if a ribozyme or other RNA motifs with extensive structure, are introduced due to steric hindrance and the high concentrations of cations needed to enable ribozyme activity.

Provided herein is a novel RNA-based system, and methods of production thereof, to generate diverse members of the Orthomyxoviridae family, independent of DNA plasmids, based on the incorporation of primary microRNA (pri-miRNA) hairpin structures at the 5′ end of the vRNA segments. While the total number of segments, gene organization between segments, and coding material of non-RdRp or NP products can vary amongst the Orthomyxoviridae family, the methodologies outlined here are not impacted by these differences and could thus be applied to all genera in the family. As transfection of RNA only requires cytoplasmic entry, this methodology is not restricted to available transfectable cell lines nor knowledge of species-specific promoter requirements. Therefore, the invention of the present application not only extends virus systems for diverse vectors, it enables their scalability and potential therapeutic use by leveraging non-mammalian systems already used to produce FDA-approved biologics.

Also provided herein are methods of disease treatment and prevention using the novel RNA-based systems. For example, the RNA-based systems of the present application may be used to deliver exogenous material including desired open reading frames (ORFs) or non-coding RNA (ncRNA).

### A. Definitions

The term “IVT-vRNA” refers to in vitro transcribed (IVT) RNA molecule comprising (i) a miRNA hairpin structure at the 5′ end and (ii) a viral RNA (“vRNA”), and optionally, (iii) a miRNA hairpin structure at the 3′ end. In some embodiments, the miRNA hairpin structure(s) present in IVT-vRNA is(are) cleaved in a host cell comprising a miRNA processing machinery (e.g., the RNase III nuclease Drosha) resulting in a vRNA with the precise terminal ends recognized by the cognate viral RNA-dependent RNA polymerase (RdRp).

In some embodiments, individual bases in a given miRNA hairpin can be changed or modified so long as the overall structure is maintained.

The term “influenza virus” is used herein to define a viral species of the Orthomyxoviridae family. The genera of the Orthomyxoviridae family can include Alphainfluenzavirus, Betainfluenzavirus, Gammainfluenzavirus, Deltainfluenzavirus, Isavirus, Thogotovirus, and Quaranjavirus. The term influenza is meant to include any strain or serotype of the influenza virus, including any combination of HA, e.g., H1, H2, H3, H4, H5, H6, H7, H8, H9, H10, H11, H12, H13, H14, H15, H16; and NA, e.g., N1, N2, N3, N4, N5, N6, N7, N8 or N9 genes in addition to other non-human genera.

In the context of the present disclosure, “coding region” refers to areas of viral RNA which encode amino acids that are represented in translated proteins.

The terms “microRNA” or “miRNA” as used herein refer to a structured RNA that is processed by the Rnase III components of the host cell. The resulting product is a ˜19-25 base pair endogenous single stranded RNA that regulates the expression of target mRNAs via a 7 base pair “seed” sequence (i.e., sequence at 5′ positions 1-7 or 2-8 of miRNA). Complementarity of an mRNA sequence to the “seed” is normally found in the 3′ untranslated region (3′ UTR). Bartel, Cell 116(2):281 (2004). miRNA regulation moderately affects global protein production resulting in a “fine tuning” of the cellular transcriptome. Baek et al., Nature 455(7209):64 (2008) and Selbach et al., Nature 455(7209):58 (2008).

The term “transfection” as used herein refers to the introduction or delivery of materials, such as purified nucleic acids (e.g., DNA or RNA), into cells. The transfection is often performed through methods that do not involve viral infection. The RNA-based systems provided herein are transfected into cells, for example, by lipid nanoparticles or non-lipid based delivery vehicles.

In some aspects, provided herein is an RNA-based system for generating a virus of the Orthomyxoviridae family. In some embodiments, the RNA-based system comprises (i) one or more mRNAs encoding an RNA-dependent RNA polymerase (RdRp) and a nucleoprotein of the virus; and, (ii) a plurality of in vitro transcribed (“IVT”) viral RNA (“IVT-vRNA”) collectively comprising the genome of the virus, wherein each IVT-vRNA of the plurality of IVT-vRNAs comprise a genomic segment of the virus (vRNA), wherein each IVT-vRNA is modified to comprise a miRNA hairpin structure at the 5′ end, and optionally the 3′ end, of the vRNA comprised therein, and wherein the miRNA hairpin structure(s) in the IVT-vRNA can be cleaved in a host cell comprising a miRNA processing machinery to produce the vRNA with the termini that are recognizable by the cognate viral RdRp for transcription and replication. In the context of the present invention, the term “rescue” or “rescuing” is used interchangeably with “generate” or “generating” and “produce” or “producing”.

The term “complementarity” means that a nucleic acid can form hydrogen bond(s) with another nucleic acid sequence by either traditional Watson-Crick or other non-traditional types of interactions such as Wobble-base pairing which permits binding of guanine and uracil. A percent complementarity indicates the percentage of residues in a nucleic acid molecule that can form hydrogen bonds with a second nucleic acid sequence.

In reference to the nucleic acid molecules of the present invention, the binding free energy for a nucleic acid molecule with its complementary sequence is sufficient to allow the relevant function of the nucleic acid to proceed, e.g., miRNA activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J Am. Chem. Soc. 109:3783-3785). “Perfectly complementary” means that all the contiguous residues of a nucleic acid sequence will hydrogen bond with the same number of contiguous residues in a second nucleic acid sequence. In one embodiment, the human miRNA has partial complementarity (i.e., less than 100% complementarity) with the corresponding target influenza nucleic acid molecule.

An “individual” or “subject” or “animal”, as used herein, refers to vertebrates or invertebrates that support a negative strand RNA virus infection, specifically Orthomyxoviridae infections, including, but not limited to, birds (such as water fowl and chickens) and members of the mammalian species, such as canine, feline, lupine, mustela, rodent (racine, murine, etc.), equine, bovine, ovine, caprine, porcine species, and primates, the latter including humans. In one embodiment, the subject is a human. In other embodiments, the subject is an insect.

The term “about” or “approximately” means within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

As used herein, “percent (%) sequence identity” with respect to a nucleic acid sequence are defined as the percentage of nucleic acid residues in a candidate sequence that are identical with the nucleic acid residues in the specific nucleic acid sequence, after aligning the sequences and introducing gaps, if necessary, to achieve the maximum percent sequence identity, and not considering any conservative substitutions as part of the sequence identity. Alignment for purposes of determining percent sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter.

As used herein and in the appended claims, the singular forms “a,” “an,” and “the” include plural referents unless the context clearly dictates otherwise.

### B. RNA-Based System

Provided herein is an RNA-based system for generating a virus of the Orthomyxoviridae family. The generated virus may be replication competent, conditionally active (e.g., conditionally competent), or defective. This system leverages in vitro transcription (IVT) of self-processing precursor viral RNA (vRNA) templates (e.g., IVT-vRNAs) alongside mRNA encoding PB2, PB1, PA, and NP to directly generate recombinant viruses from the Orthomyxoviridae family in the absence of plasmid transfection or helper virus. The flexibility of the RNA-based system described herein permits access to an array of viral vector delivery systems with the potential to efficiently deliver therapeutic cargoes, such as, e.g., a desired ORF or ncRNA, to the respiratory tract for the treatment of diseases such as, e.g., lung cancer, asthma, COPD, pulmonary fibrosis, or as a vaccine platform.

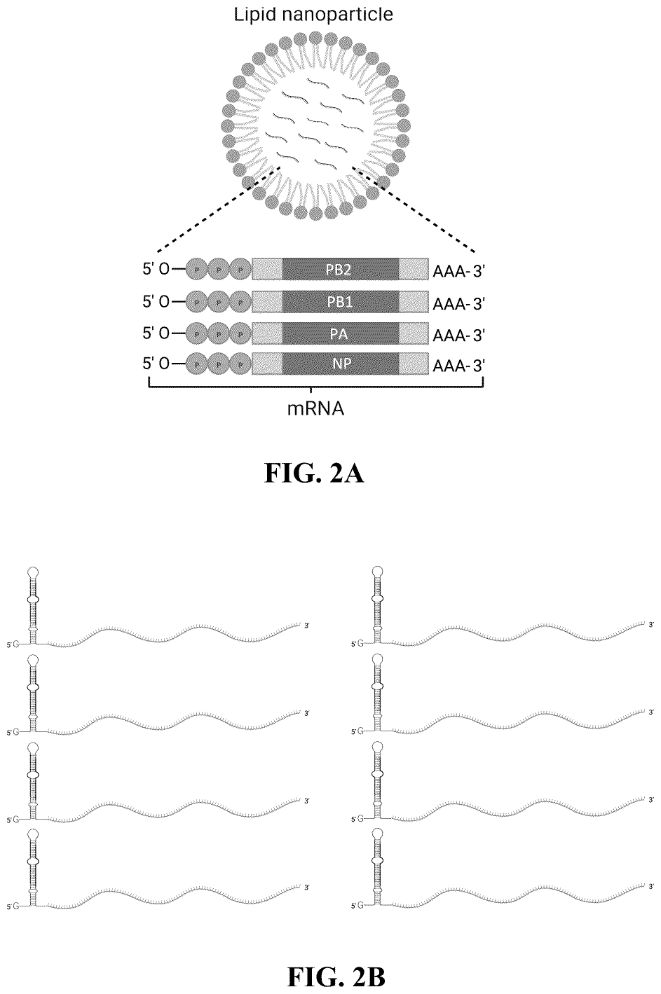

As shown in FIGS. 2A-2B, the RNA-based systems of the present invention comprise an RNA-dependent RNA polymerase (RdRp) and a nucleoprotein of the virus, and a plurality of vRNA segments collectively comprising the genome of the virus. The RdRp, which comprises three open reading frames of the core components of PB2, PB1, and PA, and the nucleoprotein (NP), may be encoded by IVT mRNA that is capped at the 5′ end and polyadenylated at the 3′ end (FIG. 2A). In some embodiments, IVT comprises the template-directed synthesis of mRNA molecules using a polymerase. The genome of an Alphainfluenza virus of the Orthomyxoviridae family may be encoded on 8, or fewer, transcriptionally active vRNA segments (e.g., segment 1 vRNA for PB2; segment 2 vRNA for PB1; segment 3 vRNA for PA; segment 4 vRNA for HA; segment 5 vRNA for NP; segment 6 vRNA for NA; segment 7 vRNA for M1 and M2; and segment 8 vRNA for NS1 and NS2), which are modified to comprise a mircroRNA (miRNA) hairpin structure at the 5′ end, and optionally the 3′ end, of the vRNA comprised therein (FIG. 2B). One of skill in the art would readily appreciate that other genera of the Orthomyxoviridae family encode different ORFs on a different number of segments, compared to the genome of an Alphainfluenza virus. Due to the ubiquitous nature of miRNA biological machinery across cells, the miRNA hairpin structures are subsequently cleaved in a host cell to produce the vRNA comprised therein, thereby allowing rescue of a virus of the Orthomyxoviridae family directly following the introduction of IVT vRNA and/or IVT mRNA.

Thus, in some aspects, provided herein is an RNA-based system for rescuing a virus of the Orthomyxoviridae family, the system comprising: (i) a RdRp and a nucleoprotein of the virus; and, (ii) a plurality of IVT-vRNAs collectively comprising the genome of the virus, wherein each IVT-vRNA of the plurality of IVT-vRNAs comprise a genomic segment of the virus (e.g., a transcriptionally active genomic segment), wherein each IVT-vRNA is modified to comprise a miRNA hairpin structure at the 5′ end, and optionally the 3′ end, of the vRNA comprised therein, and wherein the miRNA hairpin structure(s) in the IVT-vRNA can be cleaved in a host cell comprising a miRNA processing machinery to produce the vRNA with the termini that are recognizable by the cognate viral RdRp for transcription and replication. In some embodiments, at least one IVT-vRNA of the plurality of IVT-vRNAs is modified to comprise a miRNA hairpin structure at the 3′ end, which can then additionally be polyadenylated. In some embodiments, the miRNA hairpin structure is derived from naturally occurring primary miRNAs (pri-miRNAs) or miRNAs whose sequence is modified but whose secondary structure is maintained to ensure accurate processing. In some embodiments, the miRNA hairpin structure is derived from pri-miR-16. In some embodiments, the miRNA hairpin structure comprises the nucleic acid sequence set forth in SEQ ID NO: 15, or a nucleic acid sequence having at least about 80% (e.g., at least any of about 80%, 85%, 90%, 91%, 92%, 93%, 94%, 95%, 96%, 97%, 98%, or 99%) sequence identity to the nucleic acid sequence set forth in SEQ ID NO: 15. In some embodiments, the miRNA hairpin structure is derived from pri-miR-21a. In some embodiments, the miRNA hairpin structure comprises the nucleic acid sequence set forth in SEQ ID NO: 16, or a nucleic acid sequence having at least about 80% (e.g., at least any of about 80%, 85%, 90%, 91%, 92%, 93%, 94%, 95%, 96%, 97%, 98%, or 99%) sequence identity to the nucleic acid sequence set forth in SEQ ID NO: 16.

In some embodiments, the RdRp comprises PB2, PB1, and PA. In some embodiments, the nucleoprotein comprises NP. In some embodiments, the plurality of IVT-vRNAs comprise IVT-vRNA 1 for PB2, IVT-vRNA 2 for PB1, IVT-vRNA 3 for PA, IVT-vRNA 4 for HA, IVT-vRNA 5 for NP, IVT-vRNA 6 for NA, IVT-vRNA 7 for M1 and M2, and IVT-vRNA 8 for NS1 and NS2. In some embodiments, at least one of the IVT-vRNAs comprise a heterologous nucleic acid sequence. In some embodiments, the virus of the Orthomyxoviridae family is an influenza virus.

In some embodiments, provided herein is an RNA-based system for rescuing a virus of the Orthomyxoviridae family, the system comprising: (i) one or more mRNAs encoding a RdRp and a nucleoprotein of the virus; and, (ii) a plurality of IVT-vRNAs collectively comprising the genome of the virus, wherein each IVT-vRNA of the plurality of IVT-vRNAs comprise a genomic segment of the virus (vRNA), wherein each IVT-vRNA is modified to comprise a miRNA hairpin structure at the 5′ end, and optionally the 3′ end, of the vRNA with the termini that are recognizable by the cognate viral RdRp for transcription and replication, and wherein the miRNA hairpin structure(s) in the IVT-vRNA can be cleaved in a host cell comprising a miRNA processing machinery to produce the vRNA comprised therein. In some embodiments, at least one IVT-vRNA of the plurality of IVT-vRNAs is modified to comprise a miRNA hairpin structure at the 3′ end. In some embodiments, at least one IVT-vRNA is modified to comprise a miRNA hairpin structure at the 3′ end further comprises a poly A sequence at the 3′ end of the miRNA hairpin structure. In some embodiments, the miRNA hairpin structure is derived from pri-miR-16. In some embodiments, the miRNA hairpin structure comprises the nucleic acid sequence set forth in SEQ ID NO: 15, or a nucleic acid sequence having at least about 80% (e.g., at least any of about 80%, 85%, 90%, 91%, 92%, 93%, 94%, 95%, 96%, 97%, 98%, or 99%) sequence identity to the nucleic acid sequence set forth in SEQ ID NO: 15. In some embodiments, the miRNA hairpin structure is derived from pri-miR-21a. In some embodiments, the miRNA hairpin structure comprises the nucleic acid sequence set forth in SEQ ID NO: 16, or a nucleic acid sequence having at least about 80% (e.g., at least any of about 80%, 85%, 90%, 91%, 92%, 93%, 94%, 95%, 96%, 97%, 98%, or 99%) sequence identity to the nucleic acid sequence set forth in SEQ ID NO: 16. In some embodiments, the one or more mRNAs comprise a 5′ cap. In some embodiments, the one or more mRNAs comprise a 3′ polyA signal. In some embodiments, the one or more mRNAs are on separate nucleic acid molecules. In some embodiments, the one or more mRNAs are provided in a single nucleic acid molecule. In some embodiments, the one or more mRNAs encode PB2, PB1, PA, and NP. In some embodiments, the plurality of IVT-vRNAs comprise IVT-vRNA 1 for PB2, IVT-vRNA 2 for PB1, IVT-vRNA 3 for PA, IVT-vRNA 4 for HA, IVT-vRNA 5 for NP, IVT-vRNA 6 for NA, IVT-vRNA 7 for M1 and M2, and/or IVT-vRNA 8 for NS1 and NS2. In some embodiments, at least one of the IVT-vRNAs comprise a heterologous nucleic acid sequence. In some embodiments, the virus of the Orthomyxoviridae family is an influenza virus.

In some embodiments, provided herein is an RNA-based system for rescuing a virus of the Orthomyxoviridae family, the system comprising: (i) one or more mRNAs encoding a RdRp and a nucleoprotein of the virus; and, (ii) a plurality of IVT-vRNAs collectively comprising the genome of the virus, wherein each IVT-vRNA of the plurality of IVT-vRNAs comprise a genomic segment of the virus, wherein each IVT-vRNA is modified to comprise a miRNA hairpin structure at the 5′ end, and optionally the 3′ end, of the vRNA comprised therein, wherein the miRNA hairpin structure in the IVT-vRNA can be cleaved in a host cell comprising a miRNA processing machinery to produce the vRNA with the termini that are recognizable by the cognate viral RdRp for transcription and replication, and wherein the one or more mRNAs and the plurality of IVT-vRNAs are generated by IVT (e.g., IVT mRNA and IVT-vRNA). In some embodiments, the one or more IVT mRNAs and the plurality of IVT-vRNAs are in vitro transcribed using the T7 promoter. In some embodiments, the one or more IVT mRNAs comprise the nucleic acid sequence set forth in any one of SEQ ID NOs: 10-13. In some embodiments, the plurality of IVT vRNAs comprise the nucleic acid sequence set forth in any one of SEQ ID NOs: 2-9. In some embodiments, at least one of the IVT-vRNAs comprise a heterologous nucleic acid sequence. In some embodiments, the virus of the Orthomyxoviridae family is an influenza virus.

In some embodiments, provided herein is an RNA-based system, the system comprising: (i) one or more mRNAs encoding a RdRp and a nucleoprotein of the virus; and, (ii) a plurality of IVT-vRNAs collectively comprising the genome of the virus, wherein each IVT-vRNA of the plurality of IVT-vRNAs comprise a genomic segment of the virus, wherein each IVT-vRNA is modified to comprise a miRNA hairpin structure at the 5′ end, and optionally the 3′ end, of the vRNA comprised therein, wherein the miRNA hairpin structure in the IVT-vRNA can be cleaved in a host cell comprising a miRNA processing machinery to produce the vRNA with the termini that are recognizable by the cognate viral RdRp for transcription and replication, and wherein at least one of the IVT-vRNAs comprise a heterologous nucleic acid sequence. In some embodiments, at least one of the IVT-vRNAs comprise a heterologous nucleic acid sequence. In some embodiments, at least two of the IVT-vRNAs comprise a heterologous nucleic acid sequence. In some embodiments, the IVT-vRNA comprising a heterologous nucleic acid sequence comprises IVT-vRNA 1 for PB2, IVT-vRNA 2 for PB1, IVT-vRNA 3 for PA, IVT-vRNA 4 for HA, IVT-vRNA 5 for NP, IVT-vRNA 6 for NA, IVT-vRNA 7 for M1 and M2, and/or IVT-vRNA 8 for NS1 and NS2. In some embodiments, the heterologous nucleic acid sequence is inserted at a 5′ non-coding region of the vRNA. In some embodiments, the heterologous nucleic acid sequence is inserted at a 3′ non-coding region of the vRNA. In some embodiments, the heterologous nucleic acid sequence comprises a complement sequence of a coding sequence encoding a recombinant protein. In some embodiments, the heterologous nucleic acid has a length of about 800 to about 4000 nucleotides. In some embodiments, at least one IVT-vRNAs of the plurality of IVT-vRNAs is modified to comprise a miRNA hairpin structure at the 3′ end. In some embodiments, the at least one IVT-vRNA modified to comprise a miRNA hairpin structure at the 3′ end further comprises a poly A sequence 3′ to the miRNA hairpin structure. In some embodiments, the one or more mRNAs comprise a 5′ cap. In some embodiments, the one or more mRNAs comprise a 3′ polyA signal. In some embodiments, the one or more mRNAs are on separate nucleic acid molecules. In some embodiments, the one or more mRNAs are provided in a single nucleic acid molecule. In some embodiments, the one or more mRNAs encode PB2, PB1, PA, and NP. In some embodiments, the plurality of vRNA segments comprise IVT-vRNA 1 for PB2, IVT-vRNA 2 for PB1, IVT-vRNA 3 for PA, IVT-vRNA 4 for HA, IVT-vRNA 5 for NP, IVT-vRNA 6 for NA, IVT-vRNA 7 for M1 and M2, and/or IVT-vRNA 8 for NS1 and NS2. In some embodiments, at least one of the IVT-vRNAs comprise a heterologous nucleic acid sequence. In some embodiments, the virus of the Orthomyxoviridae family is an influenza virus.

In some embodiments, cleavage of the miRNA hairpin structure(s) in the plurality of IVT-vRNAs in a host cell comprising a miRNA processing machinery produces transcriptionally active vRNAs.

In some embodiments, the generated virus is replication competent, conditionally competent, or defective.

i. Viral Components
a. Orthomyxoviridae Virus Family

In some aspects, the RNA-based systems provided herein are useful for rescuing a virus of the Orthomyxoviridae family. The generated virus may be replication competent or conditionally active (e.g., conditionally competent), or defective. Orthomyxoviridae are a group of related, yet antigenically and genetically diverse viruses. In mammals, such as humans, Orthomyxoviridae cause respiratory tract infections that can range from mild to lethal.

More specifically, Orthomyxoviridae is a family of virus that encode their genome in segments of negative-sense RNA. In some embodiments, Orthomyxoviridae contain six to eight segments of linear negative-sense single stranded RNA. They have a total genome length that is between about 10,000 and about 14,600 nucleotides.

The Orthomyxoviridae family comprises seven genera: Alphainfluenzavirus, Betainfluenzavirus, Gammainfluenzavirus, Deltainfluenzavirus, Isavirus, Thogotovirus, and Quaranjavirus. Alphainfluenzaviruses, betainfluenzaviruses, gammainfluenzavirused, and Deltainfluenzaviruses are viruses that cause influenza in birds and mammals (e.g., humans); isavirususes infect salmon; thogotoviruses are arboviruses, infecting vertebrates and invertebrates (e.g., ticks and mosquitoes); and quaranjaviruses are arboviruses, infecting vertebrates (e.g., birds) and invertebrates (e.g., arthropods). In particular, Alphainfluenzavirus infects humans, other mammals, and birds, Betainfluenzavirus infects humans and seals, Gammainfluenzavirus infects humans and pigs, and Deltainfluenzavirus infects pigs and cattle. The RNA systems and methods provided herein can be used with any genera of the Orthomyxoviridae family.

In some embodiments, the virus of the Orthomyxoviridae family is an influenza virus (e.g., an Alphainfluenzavirus, Betainfluenzavirus, Gammainfluenzavirus, or a Deltainfluenzavirus). There are four type of influenza viruses: Type A (IAV), Type B (IBV), Type C (ICV), and Type D (IDV). Of the four types of influenza virus, three types (A, B, and C) affect humans. In some embodiments, the virus is IAV, IBV, ICV, IDV, or a variant, subtype, or reassortant thereof.

IAV may be categorized by the major viral surface proteins present, hemagglutinin (HA or H) and neuraminidase (NA or N). At present, there are 18 different Has and 11 different Nas, resulting in various subtype or strain combinations of the viral surface proteins. In some embodiments, the influenza virus comprises a HA antigen selected from the group consisting of H1, H2, H3, H5, H6, H7, H9, and H10, or a variant or reassortant thereof. In some embodiments, the influenza virus comprises a NA antigen selected from the group consisting of N1, N2, N3, N7, N8, and N9, or a variant or reassortant thereof. In some embodiments, the influenza virus is selected from the group consisting of HIN1, HIN2, H2N2, H3N2, H3N8, H5N1, H5N9, H6N1, H7N2, H7N3, H7N7, H7N9, H9N2, H10N7, or a variant or reassortant thereof.

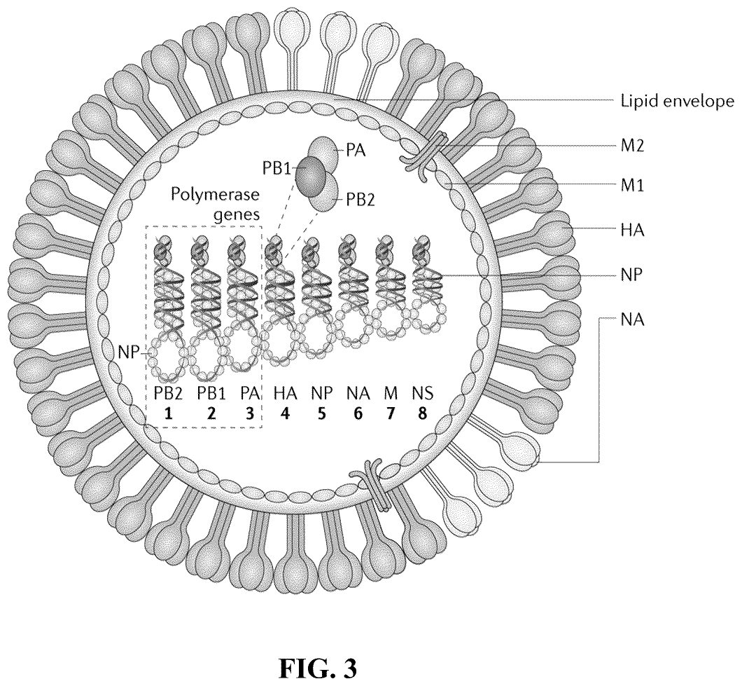

As shown in FIG. 3, IAV is comprised of a segmented genome comprising 8 negative-sense vRNAs, making up the entire viral genome of IAV. The negative-sense vRNAs are the complement sequence to their open reading frames (ORFs). Segment 1-6 generate one major protein each, whereas segment 7-8 undergo splicing to make two proteins per vRNA segment. Generation of IAV mRNA demands the viral RNA-dependent RNA polymerase (RdRp), which recognizes the terminal ends of each vRNA, and comprises three subunits (PA, PB1 and PB2). The segments of IAV illustrated in FIG. 3 are as follows: segment 1-PB2 (core RdRp component); segment 2-PB1 (core RdRp component); segment 3-PA (core RdRp component); segment 4-HA (viral attachment protein); segment 5-nucleoprotein (“NP,” RNA binding protein for viral segments); segment 6-NA (viral attachment protein); segment 7-matrix protein 1 (“M1,” virion structural protein) and matrix protein 2 (“M2,” ion channel required for entry); and, segment 8-non-structural protein 1 (“NS1,” antagonist of host antiviral defenses) and non-structural protein 2 (“NS2,” RdRp modulation and virus egress). Viruses of different genera of the Orthomyxoviridae family may have different ORFs encoding their viral components, and different vRNA segments in their genome comprising such ORFS (i.e., less than or more than 8 vRNA segments).

In some embodiments, the virus of the Orthomyxoviridae family comprises a genome comprising a plurality of vRNA segments (e.g., a segmented viral genome). In some embodiments, the plurality of vRNA segments comprise 8 or more vRNA segments. In some embodiments, each vRNA segment of the plurality of vRNA segments encodes one protein. In some embodiments, each vRNA segment of the plurality of vRNA segments may encode one or more proteins. In some embodiments, at least two of the vRNA segments each encode two proteins. In some embodiments, the plurality of vRNA segments comprise segment 1 vRNA for PB2, segment 2 vRNA for PB1, segment 3 vRNA for PA, segment 4 vRNA for HA, segment 5 vRNA for NP, segment 6 vRNA for NA, segment 7 vRNA for M1 and M2, and segment 8 vRNA for NS1 and NS2. In some embodiments, the plurality of vRNA segments comprise at least 4 vRNA segments. In some embodiments, the plurality of vRNA segments comprise, segment 1 vRNA for PB2, segment 2 vRNA for PB1, segment 3 vRNA for PA, and segment 5 vRNA for NP. In some embodiments, the plurality of vRNA segments do not comprise less than 8 vRNA segments. In some embodiments, the plurality of vRNA segments comprise collectively comprise the genome of the virus of the Orthomyxoviridae family.

In some embodiments, the virus is self-propagating. In some embodiments, the self-propagation of the virus is attenuated (e.g., limited). In some embodiments, the viral self-propagation is stopped. In some embodiments, the virus is self-propagating in particular cells, but is not self-propagating in other cells. For example, the virus may be self-propagating in an insect cell, a plant cell, or a bird cell, but not self-propagating in mammalian cells (e.g., human cells). In some embodiments, the virus is self-propagating insect cells but not self-propagating in human cells. In some embodiments, the virus is self-propagating plant cells but not self-propagating in human cells. In some embodiments, the virus is self-propagating bird cells but not self-propagating in human cells. Additional information may be found in, for example, US20120148622, the contents of which are incorporated by references in its entirety.

In some embodiments, the virus is a conditionally competent virus. In some embodiments, the virus is a partially functional virus. For example, the virus may be replication competent in a certain species or under certain conditions (e.g., the virus is replication competent at a particular temperature, but not at other temperatures). Methods of generating a conditionally competent virus are described, for example, in U.S. Pat. No. 8,986,705 B2, the contents of which are incorporated herein by reference in its entirety.

b. RdRp and Nucleoprotein

In some embodiments, the RNA-based system provided herein comprises a RdRp and a nucleoprotein (NP) of the virus of the Orthomyxoviridae family. The RdRp and nucleoprotein may be used to bind and amplify the plurality of vRNA segments of the virus.

In some embodiments, the system comprises any sequence that results in a RdRp protein and a nucleoprotein (NP) of the virus. In some embodiments, the system comprises one or more proteins, DNAs, and/or mRNAs encoding a RdRp and nucleoprotein of the virus. In some embodiments, the system comprises a protein expressing a RdRp of the virus. In some embodiments, the system comprises a protein expressing a nucleoprotein of the virus. In some embodiments, the system comprises a protein expressing a RdRp of the virus and a protein expressing a nucleoprotein of the virus. In some embodiments, the protein expressing the RdRp comprises PB2, PB1, and PA. In some embodiment, the protein expressing the nucleoprotein comprises NP. In some embodiments, the system comprises a protein expressing a RdRp of the virus and one or more mRNAs encoding a nucleoprotein of the virus. In some embodiments, the system comprises one or more mRNAs encoding a RdRp of the virus and protein expressing a nucleoprotein of the virus. In some embodiments, the one or more mRNAs and the protein comprise PB2, PB1, PA, and NP. In some embodiments, the system comprises one or more mRNAs encoding a RdRp and a nucleoprotein of the virus. In some embodiments, the RdRp and the nucleoprotein comprise PB2, PB1, PA, and NP. In some embodiments, the RdRp comprises PB2, PB1, and PA. In some embodiments, the nucleoprotein comprises NP. In some embodiments, the one or more mRNAs encode PB2, PB1, PA, and NP.

In some embodiments, the RdRp is encoded on a single nucleic acid molecule (e.g., a single DNA or a single mRNA). In some embodiments, the RdRp is encoded on more than one nucleic acid molecule. In some embodiments, the nucleoprotein is encoded on a single nucleic acid molecule (e.g., a single mRNA). In some embodiments, the nucleoprotein is encoded on more than one nucleic acid molecule. In some embodiments, the RdRp and the nucleoprotein are encoded on the same nucleic acid molecule. In some embodiments, the RdRp and the nucleoprotein are encoded on separate nucleic acid molecules.

In some embodiments, the one or more mRNAs are generated by IVT (i.e., IVT-mRNA). In embodiments, the IVT-mRNA sequences are codon optimized. In some embodiments, the one or more IVT-mRNAs comprise an IVT-mRNA comprising the open reading frame of PB2, PB1, PA, or NP. In embodiments, the one or more IVT-mRNAs comprise the nucleic acid sequence of any one of SEQ ID NOs: 10-13. In some embodiments, the IVT-mRNA comprises the nucleic acid sequence of SEQ ID NO: 10, or a nucleic acid sequence comprising at least about 80% sequence identity with the nucleic acid sequence set forth in SEQ ID NO: 10. In some embodiments, the IVT-mRNA comprises the nucleic acid sequence of SEQ ID NO: 11, or a nucleic acid sequence comprising at least about 80% sequence identity with the nucleic acid sequence set forth in SEQ ID NO: 11. In some embodiments, the IVT-mRNA comprises the nucleic acid sequence of SEQ ID NO: 12, or a nucleic acid sequence comprising at least about 80% sequence identity with the nucleic acid sequence set forth in SEQ ID NO: 12. In some embodiments, the IVT-mRNA comprises the nucleic acid sequence of SEQ ID NO: 13, or a nucleic acid sequence comprising at least about 80% sequence identity with the nucleic acid sequence set forth in SEQ ID NO: 13.

### C. IVT-vRNAs

In some embodiments, the RNA-based system provided herein comprises a plurality of IVT-vRNAs collectively comprising the genome of the virus of the Orthomyxoviridae family (e.g., each IVT-vRNA is a vRNA segment comprising a miRNA hairpin structure, generated by IVT). In some embodiments, each IVT-vRNA of the plurality of IVT-vRNAs is transcriptionally active. In some embodiments, a portion of each IVT-vRNA of the plurality of IVT-vRNAs is transcriptionally active.

In some embodiments, each IVT-vRNA of the plurality of IVT-vRNAs comprise a transcriptionally active genomic segment of the virus. In some embodiments, each IVT-vRNA comprises an open reading frame of a transcriptionally active genomic segment of the virus. In some embodiments, the system comprises less than 8 IVT-vRNAs. In some embodiments, the system comprises 8 IVT-vRNAs. In some embodiments, each IVT-vRNA of the plurality of IVT-vRNAs encodes one protein. In some embodiments, each IVT-vRNA of the plurality of IVT-vRNAs may encode one or more proteins. In some embodiments, at least two of the IVT-vRNAs each encode two proteins. In some embodiments, the plurality of IVT-vRNAs comprise IVT-vRNA 1 for PB2, IVT-vRNA 2 for PB1, IVT-vRNA 3 for PA, IVT-vRNA 4 for HA, IVT-vRNA 5 for NP, IVT-vRNA 6 for NA, IVT-vRNA 7 for M1 and M2, and/or IVT-vRNA 8 for NS1 and NS2. In some embodiments the plurality of IVT-vRNAs comprise at least 4 IVT-vRNAs. In some embodiments, the plurality of IVT-vRNAs comprise at least IVT-vRNA 1 for PB2, IVT-vRNA 2 for PB1, IVT-vRNA 3 for PA, and IVT-vRNA 5 for NP. In some embodiments, the plurality of IVT-vRNAs comprise less than 8 IVT-vRNAs, but comprise at least IVT-vRNA 1 for PB2, IVT-vRNA 2 for PB1, IVT-vRNA 3 for PA, and IVT-vRNA 5 for NP. In some embodiments, the plurality of IVT-vRNAs collectively comprise the genome of the virus of the Orthomyxoviridae family. In some embodiments, the IVT-vRNAs are codon optimized.

In some embodiments, each IVT-vRNA of the plurality of IVT-vRNAs does not have a 3′ poly A sequence. In some embodiments, each IVT-vRNA of the plurality of IVT-vRNAs does not have a 5′ cap. In some embodiments, each IVT-vRNA of the plurality of IVT-vRNAs has an ‘A’ or a ‘U’ base at its 5′ terminus. In some embodiments, each IVT-vRNA of the plurality of IVT-vRNAs has a ‘G’ base at its 5′ terminus.

In some embodiments, the IVT-vRNAs may be shifted, replaced, and/or redesigned to fulfill the purpose of the RNA-based system. For example, some encoded proteins may be affixed to a different IVT-vRNA to make the IVT-vRNA they previous occupied available for use (e.g., available for cargo). By way of illustration, in some embodiments, NS1 and NS2 may be moved from IVT-vRNA 8 for NS1 and NS2 to a different IVT-vRNA, thereby freeing IVT-vRNA 8 for encoding cargo of the RNA-based system. In another example, IVT-vRNA 4 and IVT-vRNA 6, encoding viral surface attachment proteins HA and NA, respectively, may be replaced with rhabdovirus VSV G to enable viral entry and egress while providing an unused segment for cargo. One of skill in the art would readily understand that there are infinite ways to shift, replace, and/or redesign the IVT-vRNAs to provide an unused IVT-vRNA for purposes of carrying cargo within the RNA-based system.

An IVT-vRNA of the plurality of IVT-vRNAs may comprise one or more sequences or features in addition to an IVT-vRNA (e.g., any of the IVT-vRNAs described herein). In some embodiments, the IVT-vRNA comprises a 3′ non-coding region. In some embodiments, the IVT-vRNA comprises a 5′ non-coding region. In some embodiments, the IVT-vRNA comprises a 3′ non-coding region and a 5′ non-coding region.

Production of the IVT-vRNAs is further described in Section E, below. In some embodiments, the IVT-vRNAs are generated by IVT. In some embodiments, the IVT-vRNAs comprise a promoter sequence (e.g., a T7 promoter sequence) and a sequence encoding the vRNA. In some embodiments, the IVT-vRNAs comprise the nucleic acid sequence set forth in any one of SEQ ID NOs: 2-9. In some embodiments, at least one IVT-vRNA of the plurality of IVT-vRNAs comprises the nucleic acid sequence set forth in SEQ ID NO: 2, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 2. In some embodiments, at least one IVT-vRNA of the plurality of IVT-vRNAs comprises the nucleic acid sequence set forth in SEQ ID NO: 3, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 3. In some embodiments, at least one IVT-vRNA of the plurality of IVT-vRNAs comprises the nucleic acid sequence set forth in SEQ ID NO: 4, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 4. In some embodiments, at least one IVT-vRNA of the plurality of IVT-vRNAs comprises the nucleic acid sequence set forth in SEQ ID NO: 5, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 5. In some embodiments, at least one IVT-vRNA of the plurality of IVT-vRNAs comprises the nucleic acid sequence set forth in SEQ ID NO: 6, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 6. In some embodiments, at least one IVT-vRNA of the plurality of IVT-vRNAs comprises the nucleic acid sequence set forth in SEQ ID NO: 7, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 7. In some embodiments, at least one IVT-vRNA of the plurality of IVT-vRNAs comprises the nucleic acid sequence set forth in SEQ ID NO: 8, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 8. In some embodiments, at least one IVT-vRNA of the plurality of IVT-vRNAs comprises the nucleic acid sequence set forth in SEQ ID NO: 9, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 9.

ii. miRNA Hairpin Structure

The success of the RNA-based system provided herein relies on the capacity of cells to recognize and process primary miRNA transcripts (pri-miRNAs) from within the cell. miRNAs are small, regulatory RNAs that may affect the translation or stability of target mRNAs, and are derived from the longer, pri-miRNA transcripts. As miRNA biology is ubiquitous in multicellular organisms, the machinery required for metabolizing the RNA intermediates involved are also conserved. Moreover, as the recognition of pri-miRNAs is structural in nature, the sequence can be modulated and still enable efficient processing. Because the miRNA hairpins are sufficient for recognition by the miRNA machinery of a host cell, adding genetic material to the 5′ or 3′ ends of the miRNA hairpin structures does not impact processing. Thus, promoter usage, 5′capping, 3′ polyadenylation, and/or other elements/motifs that might ensure high fidelity in vitro transcription may be added upstream and/or downstream of the miRNA hairpin structures as needed, as the terminal ends will be processed only following cytoplasmic entry to liberate the desired vRNA ends.

MIRNA sequences and structures may be assessed for function using various resources known in the art. In particular, miRNA hairpin structures and processed intermediates can be assessed at the internet site found at www.miRbase.org.

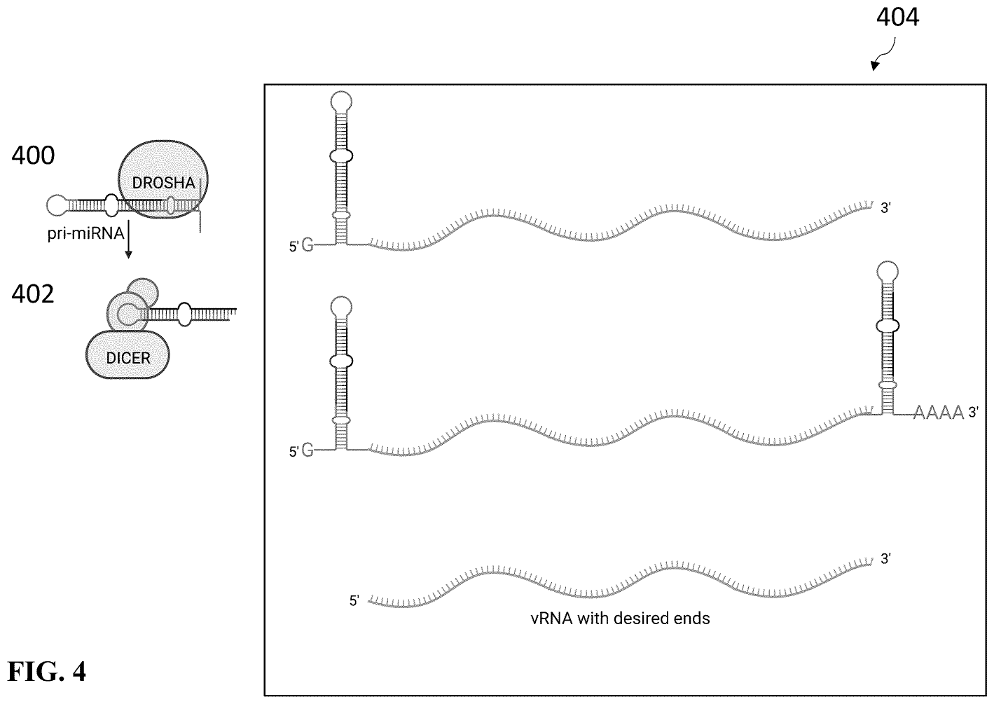

As shown in FIG. 4 in box 404, the vRNAs of the genome are modified to comprise a miRNA hairpin structure at the 5′ end, and optionally the 3′ end, of the vRNA. The miRNA hairpin structure can be amplified by recombinant polymerases. Moreover, miRNA hairpins are structurally recognized which means they can be “rewired” to maintain the shape but have a different sequence. This allows the addition of a miRNA hairpin structure to the 5′ end of the vRNA, and a full T7 promoter upstream of the hairpin structural modification. Additionally, a second miRNA hairpin structure may be optionally added to the 3′ end of the vRNA and can further include a polyA tail, for example, to aid in RNA stability. Transfection of these vRNAs, which can now be in vitro transcribed into IVT-vRNAs, can make functional vRNAs to be introduced into any cell type regardless of their transfectability profile. Each miRNA hairpin structure in the vRNA segments shown in box 404 can be cleaved in a host cell comprising a miRNA processing machinery (e.g., the Drosha and Dicer system, illustrated in FIG. 4), to produce vRNAs with the termini that are recognizable by the cognate viral RdRp for transcription and replication. Briefly, the Drosha complex crops the miRNA into a hairpin shaped pre-miRNA 400, which may then be exported to the cytoplasm for Dicer processing 402.

Thus, in some aspects, each IVT-vRNA of the plurality of the IVT-vRNAs are modified. The IVT-vRNAs may be modified such that the terminal ends of the vRNA can be precisely defined to generate a higher order structure that serves as a recognition and docking site for the RdRp. In some embodiments, the IVT-vRNAs are modified to comprise a miRNA hairpin structure. In some embodiments, the miRNA (e.g., the miRNA hairpin structure) is derived from a pri-miRNA.

In some embodiments, each IVT-vRNA is modified to comprise a miRNA hairpin structure at the 5′ end of the vRNA comprised therein. In some embodiments, each IVT-vRNA is modified to comprise a miRNA hairpin structure at the 3′ end of the vRNA comprised therein. In some embodiments, each IVT-vRNA is modified to comprise a miRNA hairpin structure at the 5′ end, and an optional miRNA hairpin structure at the 3′ end, of the vRNA comprised therein. In some embodiments, the miRNA hairpin structure at the 3′ end increases stability of the vRNA. In some embodiments, at least one of the IVT-vRNAs is modified to comprise a miRNA hairpin structure at the 3′ end. In some embodiments, the at least one IVT-vRNA modified to comprise a miRNA hairpin structure at the 3′ end further comprises a poly A sequence downstream of the miRNA hairpin structure.

The miRNA hairpin structure has been extensively described in the art, see, e.g., Adams, L. Pri-miRNA processing: structure is key. Nat Rev Genet 18, 145 (2017), which is incorporated herein by reference in its entirety. MiRNA hairpins are small regulatory RNAs that are derived from distinct primary transcripts (pri-miRNA) to yield precursor miRNA (pre-miRNAs). In particular, miRNA hairpin structures have been shown to increase the processing efficiency of pri-miRNAs. The overall structure of theses hairpins may comprise a stem region and an apical loop region. Studies have indicated an optimal hairpin stem length of ˜35 nucleotides and an optimal apical loop sizes of ˜3-23 nucleotides. In addition, the presence of some primary sequence motifs—for example, the CNNC nucleotide motif and the UG nucleotide motif, optionally within the miRNA hairpin structure-appear to enhance processing.

In some embodiments, the structure of the miRNA hairpin structure can be of any form, so long as the miRNA hairpin structure is recognized in the cell (e.g., recognized by Drosha in the cell). In some embodiments, the miRNA hairpin structure on the 3′ end has a different nucleic acid sequence from the miRNA hairpin structure on the 5′ end. In some embodiments, the miRNA hairpin structure on the 3′ end has the same nucleic acid sequence as the miRNA hairpin structure on the 5′ end. In some embodiments, the miRNA hairpin structure on the 3′ end has the same structure as the miRNA hairpin structure on the 5′ end. In some embodiments, the miRNA hairpin structure on the 3′ end has a different structure from the miRNA hairpin structure on the 5′ end. In some embodiments, the miRNA hairpin structure on the 3′ end has a different nucleic acid sequence from the miRNA hairpin structure on the 5′ end, and the miRNA hairpin structure on the 3′ end has the same structure as the miRNA hairpin structure on the 5′ end.

In some embodiments, the miRNA hairpin structure is recognized by Drosha in a host cell. In some embodiments, each miRNA hairpin structure in the vRNA segment can be cleaved to produce a vRNA. In some embodiments, each miRNA hairpin structure is cleaved in a host cell (e.g., by Drosha). In some embodiments, the host cell comprises a miRNA processing machinery. In some embodiments, the miRNA processing machinery specifically recognizes the miRNA hairpin structure. In some embodiments, the miRNA processing machinery comprises Drosha. In some embodiments, the miRNA processing machinery comprises the Drosha and Dicer system. In some embodiments, the miRNA hairpin structure is cleaved by Drosha. In some embodiments, the cleavage of the miRNA hairpin structure in a host cell comprising miRNA processing machinery produces a vRNA (e.g., which can generate a replication competent, or conditionally active (e.g., conditionally competent), or defective virus of the Orthomyxoviridae family).

In some embodiments, the miRNA hairpin structure has a stem length of about 25 to about 45 nucleotides, such as any of about 25 to about 35 nucleotides, about 30 to about 40 nucleotides, and about 35 to about 45 nucleotides. In some embodiments, the miRNA hairpin structure has a stem length of greater than about 25 nucleotides, such as great than any of about 30, 35, 40, 45, or more, nucleotides. In some embodiments, the mRNA hairpin structure has a stem length of less than about 45 nucleotides, such as less than any of about 40, 35, 30, 25, or fewer, nucleotides. In some embodiments, the mRNA hairpin structure has a stem length of any of about 25, 26, 27, 28, 29, 30, 31, 32, 33, 34, 35, 36, 37, 38, 39, 40, 41, 42, 43, 44, or 45 nucleotides.

In some embodiments, the miRNA hairpin structure has an apical loop size of about 3 to about 23 nucleotides, such as any of about 3 to about 15 nucleotides, about 10 to about 20 nucleotides, and about 12 to about 23 nucleotides. In some embodiments, the miRNA hairpin structure has an apical loop size of greater than about 3 nucleotides, such as greater than any of about 5, 6, 7, 8, 9, 10, 11, 12, 13, 14, 15, 16, 17, 18, 19, 20, 21, 22, 23, or more, nucleotides. In some embodiments, the miRNA hairpin structure has an apical loop size of less than about 23 nucleotides, such as less than any of about 22, 21, 20, 19, 18, 17, 16, 15, 14, 13, 12, 11, 10, 9, 8, 7, 6, 5, 4, 3, or fewer, nucleotides. In some embodiments, the miRNA hairpin structure has an apical loop size of any of about 3, 4, 5, 6, 7, 8, 9, 10, 11, 12, 13, 14, 15, 16, 17, 18, 19, 20, 21, 22, or 23 nucleotides.

In some embodiments, the miRNA hairpin structure comprises one or more nucleotide motifs. In some embodiments, the one or more nucleotide motifs selectively enhance the processing of optimal-length hairpins. In some embodiments, the one or more nucleotide motifs reduce the accumulation of mature miRNA in a host cell. In some embodiments, the one or more nucleotide motifs are present in the apical loop or the stem of the miRNA hairpin structure. For example, in some embodiments the miRNA hairpin structure contains a CNNC nucleotide motif, where ‘N’ represents any nucleotide. In some embodiments, the miRNA hairpin structure contains a UG nucleotide motif. In some embodiments, the UG nucleotide motif is present in the apical loop of the miRNA hairpin structure. In some embodiments, UG nucleotide motif is upstream of the apical loop of the miRNA hairpin structure. In some embodiments, the miRNA hairpin structure contains a CNNC nucleotide motif and a UG nucleotide motif. In some embodiments, the CNNC nucleotide motif is downstream (e.g., 3′) to the UG nucleotide motif.

In some embodiments, the miRNA hairpin structure is derived from a pri-miRNA.

In some embodiments, the pri-miRNA contains a miRNA hairpin structure. In some embodiments, the miRNA hairpin structure is derived from a pri-miRNA hairpin structure. In some embodiments, the pri-miRNA hairpin structure is synthetic. In some embodiments, the pri-miRNA has a high level of processing efficiency. Processing efficiency may be determined by any method known in the art, for example, RNA-Seq read captures. In some embodiments, the miRNA hairpin structure is derived from pri-miR-16. In some embodiments, the miRNA hairpin structure comprises the nucleic acid sequence set forth in SEQ ID NO: 15, or a nucleic acid sequence having at least about 80% (e.g., at least any of about 80%, 85%, 90%, 91%, 92%, 93%, 94%, 95%, 96%, 97%, 98%, or 99%) sequence identity to the nucleic acid sequence set forth in SEQ ID NO: 15. In some embodiments, the miRNA hairpin structure is derived from pri-miR-21a. In some embodiments, the miRNA hairpin structure comprises the nucleic acid sequence set forth in SEQ ID NO: 16, or a nucleic acid sequence having at least about 80% (e.g., at least any of about 80%, 85%, 90%, 91%, 92%, 93%, 94%, 95%, 96%, 97%, 98%, or 99%) sequence identity to the nucleic acid sequence set forth in SEQ ID NO: 16.

It should be understood that the present invention may encompass any alternate pri-miRNA, and that the provided RNA-based systems are not limited by the particular pri-miRNAs disclosed herein. In some embodiments, IVT constructs (e.g., IVT-vRNAs) are generated from the pri-miRNA, such as pri-miR-16 or pri-miR-21a.

iii. mRNA Features

The mRNAs provided herein (e.g., one or more mRNAs encoding a RdRp and/or a nucleoprotein of the virus) may comprise sequences or features in addition to a coding sequence encoding a RdRp and/or a nucleoprotein. These features may be used, for example, to improve targeting of the RdRp and/or nucleotide encoded by the mRNA and to increase stability of the mRNA. Exemplary features may include, but are not limited to, a polyA signal sequence and a 5′ cap, or a combination thereof. The mRNAs may further include 5′ and/or 3′ untranslated regions, chemical modifications within the mRNA sequences, and/or sequences encoding a signal peptide.

In some embodiments, the one or more mRNA comprise a polyA sequence (e.g, a polyadenylation signal sequence). PolyA sequences consist of multiple adenosine monophosphates in succession. In some embodiments, the polyA sequence is crucial for translation of the mRNA. In some embodiments, the polyA sequence is downstream of a sequence encoding a RdRp or a nucleotide in the mRNA. In some embodiments, the polyA sequence has a length of about 50 nucleotides or longer, such as about 60, 70, 80, 90, 100, 150, or longer. In some embodiments, the polyA sequence has a length of less than about 150 nucleotides, such as less than any of about 100, 90, 80, 70, 50, or fewer, nucleotides.

In some embodiments, the one or more mRNAs comprise a 5′ cap. The 5′ cap may allow for the creation of stable and mature mRNA that is able to undergo translation during protein synthesis. In some embodiments, the 5′ cap increases stability of the one or more mRNAs. In some embodiments, the 5′ cap comprises a 7-methylguanosine (m7G) moiety, a trimethylated m2′2′7G moiety, or an NAD+. In some embodiments, the 5′ cap is added to the one or more mRNAs via a 5′-5′ triphosphate linkage to the first transcribed nucleotide of the one or more mRNAs.

iv. Heterologous Nucleic Acid

In some aspects, the plurality of IVT-vRNAs for use in the RNA-based system provided herein may comprise a heterologous nucleic acid sequence. The heterologous nucleic acid sequences are derived from a different organism than a host cell in which the RNA-based system is introduced. In some embodiments, the heterologous nucleic acid is configured to express a functional, recombinant protein. In some embodiments, the heterologous nucleic acid comprises an open reading frame encoding a protein.

In some embodiments, at least one of the IVT-vRNA of the plurality of IVT-vRNAs comprise a heterologous nucleic acid sequence. In some embodiments, at least two of the IVT-vRNAs comprise a heterologous nucleic acid sequence.

In some embodiments, the IVT-vRNA comprising a heterologous nucleic acid sequence comprises IVT-vRNA 1 for PB2. In some embodiments, the IVT-vRNA comprising a heterologous nucleic acid sequence comprises IVT-vRNA 2 for PB1. In some embodiments, the IVT-vRNA comprising a heterologous nucleic acid sequence comprises IVT-vRNA 3 for PA. In some embodiments, the IVT-vRNA comprising a heterologous nucleic acid sequence comprises IVT-vRNA 4 for HA. In some embodiments, the IVT-vRNA comprising a heterologous nucleic acid sequence comprises IVT-vRNA 5 for NP. In some embodiments, the IVT-vRNA comprising a heterologous nucleic acid sequence comprises IVT-vRNA 6 for NA. In some embodiments, the IVT-vRNA comprising a heterologous nucleic acid sequence comprises IVT-vRNA 7 for M1 and M2. In some embodiments, the IVT-vRNA comprising a heterologous nucleic acid sequence comprises IVT-vRNA 8 for NS1 and NS2. In some embodiments, the IVT-vRNA comprising a heterologous nucleic acid sequence comprises IVT-vRNA 1, IVT-vRNA 2, IVT-vRNA 3, IVT-vRNA 4, IVT-vRNA 5, IVT-vRNA 6, IVT-vRNA 7, or IVT-vRNA 8, or a combination thereof. In some embodiments, the IVT-vRNA comprising a heterologous nucleic acid sequence comprises IVT-vRNA 1. In some embodiments, the IVT-vRNA comprising a heterologous nucleic acid sequence comprises IVT-vRNA 2. In some embodiments, the IVT-vRNA comprising a heterologous nucleic acid sequence comprises IVT-vRNA 3. In some embodiments, the vRNA segment comprising a heterologous nucleic acid sequence comprises IVT-vRNA 5. In some embodiments, the IVT-vRNA comprising a heterologous nucleic acid sequence comprises IVT-vRNA 8. In some embodiments, the IVT-vRNA comprising a heterologous nucleic acid sequence does not comprise any segment of vRNA of the virus.

In some embodiments, the heterologous nucleic acid sequence is inserted at a 5′ non-coding region of the vRNA. In some embodiments, the heterologous nucleic acid sequence is inserted at the 3′ non-coding region of the vRNA. In some embodiments, a first heterologous nucleic acid sequence is inserted at a 5′ non-coding region of the vRNA and a second heterologous nucleic acid sequence is inserted at the 3′ non-coding region of the vRNA.

In some embodiments, the heterologous nucleic acid sequence comprises a complement of a coding sequence encoding a recombinant protein. In some embodiments, the heterologous nucleic acid sequence comprises an open reading frame encoding a recombinant protein.

In some embodiments, the heterologous nucleic acid has a length between about 800 nucleotides to about 4000 nucleotides, such as between about 800 nucleotides to about 2000 nucleotide, between about 1000 nucleotides and about 3000 nucleotides, and between about 2000 nucleotides and about 4000 nucleotides. In some embodiments, the heterologous nucleic acid has a length of greater than about 800 nucleotides, such as greater than any of about 900, 1000, 1500, 2000, 2500, 3000, 3500, 4000, or more, nucleotides. In some embodiments, the heterologous nucleic acid has a length of less than about 4000 nucleotides, such as less than any of about 3500, 3000, 2500, 2000, 1500, 1000, 900, 800, or fewer, nucleotides.

### C. Method of Viral Production

In some aspects, provided herein is a method of producing a plurality of viruses in a cell culture using an RNA-based system, such as any of the RNA-based systems described herein. In some embodiments, the method produces a plurality of viruses of the Orthomyxoviridae family. In some embodiments, the method produces an influenza virus.

In some embodiments, provided herein is a method of producing a plurality of viruses in cell culture, the method comprising: (i) introducing an RNA-based system into a population of host cells, wherein the population of host cells is capable of supporting replication of a virus of the Orthomyxoviridae family; (ii) culturing the population of host cells; and (iii) recovering a plurality of the viruses. In some embodiments, the RNA-based system comprises: (i) one or more mRNAs encoding an RNA-dependent RNA polymerase (RdRp) and a nucleoprotein of the virus; (ii) a plurality of IVT-vRNAs collectively comprising the genome of the virus, wherein each IVT-vRNA of the plurality of IVT-vRNAs comprises a genomic segment of the virus (vRNA), wherein each IVT-vRNA is modified to comprise a miRNA hairpin structure at the 5′ end, and optionally the 3′ end, of the vRNA comprised therein, and wherein the miRNA hairpin structure in the IVT-vRNA can be cleaved in a host cell comprising a miRNA processing machinery to produce the vRNA with the termini that are recognizable by the cognate viral RdRp for transcription and replication. In some embodiments, the population of host cells are mammalian cells, insect cells, plant cells, or bird cells. In some embodiments, the RNA-based system is introduced into the population of host cells via transfection. In some embodiments, the virus is attenuated in the population of host cells. In some embodiments, the method further comprises inactivating the virus.

In some embodiments, viral production can be assessed by standard plaque assays or cell-based cytotoxicity.

In some embodiments, the RNA-based system comprises a heterologous nucleic acid sequence. Thus, in some embodiments, provided herein is a method of producing a virus comprising a heterologous nucleic acid sequence. In some embodiments, the method produces a virus of the Orthomyxoviridae family comprising a heterologous nucleic acid sequence.

In some embodiments, the method of producing a plurality of viruses in a cell culture using an RNA-based system is conducted in a population of host cells that do not transfect well with DNA. In some embodiments, the population of host cells comprises yeast cells (e.g., (Pichia pastoris) or insect cells (Spodoptera frugiperda).

### D. Methods of Treatment and Prevention

Various applications and uses of the provided RNA-based system are contemplated herein.

In some aspects, provided herein are methods of treating or preventing a disease or disorder associated with an infection by a virus (e.g., a virus of the Orthomyxoviridae family) in an individual. In some embodiments, the method of treating or preventing a disease or disorder associated with an infection by a virus in an individual comprises administering to the individual an effective amount of the virus (e.g., a replication competent or conditionally competent virus, such as a partially replication competent virus, or defective virus) produced using any of the production methods provided herein. In some embodiments, the virus is of the Orthomyxoviridae family. In some embodiments, the virus is administered by intramuscular injection, intranasal administration, intravenous infection, or inhalation administration. In some embodiments, the virus is carrying a gene (e.g., a heterologous nucleic acid sequence).

In some embodiments, the method further comprises inactivating the virus. The inactivated virus may be used, in some embodiments, to generate an attenuated (e.g., killed) virus. The virus may be inactivated by standard methods known in the art. For example, in some embodiments, the virus is inactivated by heat inactivation, chemical inactivation, and/or UV cross linking.

In some embodiments, the individual is a mammal (e.g., human, non-human primate, rat, mouse, cow, horse, pig, sheep, goat, dog, cat, etc.). In some embodiments, the individual is a human. In some embodiments, the individual is a clinical patient, a clinical trial volunteer, an experimental animal, etc. In some embodiments, the individual is suspected of having the disease (e.g., viral infection). In some embodiments, the individual is diagnosed with the disease (e.g., viral infection). In some embodiments, the individual is diagnosed with a viral infection. In some embodiments, the individual is diagnosed with a viral infection caused by a virus of the Orthomyxoviridae family. In some embodiments, the individual is diagnosed with influenza, such as IAV. In some embodiments, the individual has not been exposed to the virus of the Orthomyxoviridae family.

In some embodiments, provided herein is a method of treating or preventing a disease or disorder associated with an infection by a virus in an individual, comprising administering an attenuated vaccine to the individual, wherein the attenuated vaccine comprises an effective amount of an attenuated virus (e.g., a conditionally defective virus or vector) produced using any of the production methods provided herein. In some embodiments, the virus is of the Orthomyxoviridae family. In some embodiments, the virus is administered by intramuscular injection, intranasal administration, or inhalation administration. In some embodiments, the virus is carrying a gene (e.g., a heterologous nucleic acid sequence). In some embodiments, the attenuated vaccine comprises the RNA-based system provided herein in a lipid nanoparticle (LNP).

Efficacy of the methods of treatment or prevention can be evaluated, for example, by evaluating viral load (e.g., via detection of viral DNA), duration of survival of the individual, quality of life of the individual, viral protein expression and/or activity, and/or detection of serological antibodies against the virus of the Orthomyxoviridae family.

### E. DNA-Based Templates and Methods of Production

In some aspects, provided herein is a DNA template (e.g., vRNA precursor) for production of a vRNA segment in any of the RNA-based systems provided herein. In some embodiments, the DNA-based template comprises a complementary DNA sequence corresponding to at least one vRNA segment in the RNA-based systems provided herein. The provided DNA-based template may be used to produce at least one of a plurality of vRNA segments used in the RNA-based systems provided herein.

In some embodiments, the DNA-based template comprises a promoter, a miRNA hairpin structure, a ribosome, an end sequence (e.g., a polyA sequence), or any combination thereof. In some embodiments, the DNA-based template comprises a promoter upstream of the complementary DNA sequence. In some embodiments, the promoter is a full T7 promoter, a full T3 promoter, a full SP6 promoter, or any functionally active component thereof. In some embodiments, the promoter is a full T7 promoter. In some embodiments, the full T7 promoter is comprises the nucleic acid sequence set forth in SEQ ID NO: 14 (in which the nascent RNA begins with the underlined sequence 5′-GATAAT . . . 3′), or a nucleic acid sequencing having at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 14. In some embodiments, one or more miRNA hairpin structures are incorporated onto the DNA-based template. In some embodiments, the miRNA hairpin structure is incorporated downstream of the promoter on the DNA-based template. In some embodiments, the at least one IVT-vRNA is incorporated downstream of the miRNA hairpin structure and the promoter on the DNA-based template. In some embodiments, the at least one IVT-vRNA is incorporated in a 5′ to 3′ orientation. In some embodiment, the at least one IVT-vRNA is codon optimized. In some embodiments, the at least one IVT-vRNA comprises IVT-vRNA 1, IVT-vRNA 2, IVT-vRNA 3, IVT-vRNA 4, IVT-vRNA 5, IVT-vRNA 6, IVT-vRNA 7, and/or IVT-vRNA 8. In some embodiments, an optional second miRNA hairpin structure is incorporated downstream of the at least one IVT-vRNA. In some embodiments, any additional end sequence may be incorporated following the optional second miRNA hairpin structure.

In some embodiments, the DNA-based template comprises the nucleic acid sequence set forth in any one of SEQ ID NOs: 2-9. In some embodiments, the DNA-based template comprises the nucleic acid sequence set forth in SEQ ID NO: 2, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 2. In some embodiments, the DNA-based template comprises the nucleic acid sequence set forth in SEQ ID NO: 3, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 3. In some embodiments, the DNA-based template comprises the nucleic acid sequence set forth in SEQ ID NO: 4, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 4. In some embodiments, the DNA-based template comprises the nucleic acid sequence set forth in SEQ ID NO: 5, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 5. In some embodiments, the DNA-based template comprises the nucleic acid sequence set forth in SEQ ID NO: 6, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 6. In some embodiments, the DNA-based template comprises the nucleic acid sequence set forth in SEQ ID NO: 7, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 7. In some embodiments, the DNA-based template comprises the nucleic acid sequence set forth in SEQ ID NO: 8, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 8.

In some embodiments, the DNA-based template comprises a codon optimized sequence of an IVT-vRNA. In some embodiments, the DNA-based template comprises a codon optimized sequence of an IVT-vRNA comprising the open reading frame of PB2, PB1, PA, or NP. In some embodiments, the DNA-based template comprises the nucleic acid sequence set forth in SEQ ID NO: 10, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 10. In some embodiments, the DNA-based template comprises the nucleic acid sequence set forth in SEQ ID NO: 11, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 11. In some embodiments, the DNA-based template comprises the nucleic acid sequence set forth in SEQ ID NO: 12, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 12. In some embodiments, the DNA-based template comprises the nucleic acid sequence set forth in SEQ ID NO: 13, or a nucleic acid sequence comprising at least about 80% (e.g., at least any of about 80%, 85%, 90%, 91%, 92%, 93%, 94%, 95%, 96%, 97%, 98%, or 99%) sequence identity with the nucleic acid sequence set forth in SEQ ID NO: 13.

In some aspects, provided herein is a method of making/producing at least one of the plurality of IVT-vRNAs in an RNA-based system, such as any of the RNA-based systems provided herein, comprising subjecting a DNA-based segment, such as any of the DNA-based segments provided herein, to in vitro transcription (IVT), thereby obtaining the at least one IVT-vRNA. In some embodiments, the at least one IVT-vRNA is purified following IVT. In some embodiments, the at least one IVT-vRNA is capped (e.g., capped at the 5′ end). In some embodiments, the at least one IVT-vRNA produced by IVT is used to generate a virus. In some embodiments, the generation of the virus is independent of plasmid transfection.

### SEQUENCE LISTING

The sequences provided below are DNA sequences which can be used, e.g., in an in vitro transcription reaction, to produce the corresponding RNA sequences. A skilled artisan can readily generate the corresponding RNA sequences by replacing the thymines (T) with uracils (U).

| SEQ ID | | |
|---|---|---|
| NO | Description | Sequence |
| 1 | gBlock base | TAATACGACTCACTATAGGGATAATCTTGTTGATGCCTTAGCAG |
| | (italics represent | CACGTAAATATTGGCGTTAAGATTCTAAAATTATCTCCAGTATT |
| | insertion site for | AACTGTGCTGCTGAAGTAGAAACAAGNNNNNNCCTGCTTTTGCT |
| | remaining vRNA | TAGCTTATCAGACTGATGTTGACTGTTGAATCTCATGGCAACAG |
| | sequence) | CAGTCGATGGGCTGAGTAAAACATGGATCG |
| 2 | IVT_Seg1 | TAATACGACTCACTATAGGGATAATCTTGTTGATGCCTTAGCAG |
| | | CACGTAAATATTGGCGTTAAGATTCTAAAATTATCTCCAGTATT |
| | | AACTGTGCTGCTGAAGTAGAAACAAGGTCGTTTTTAAACAATTC |
| | | GACACTAATTGATGGCCATCCGAATTCTTTTGGTCGCTGTCTGG |
| | | CTGTCAGTAAGTATGCTAGAGTCCCGTTTTCGTTTCATTACCAA |
| | | CACTACGTCCCCTTGCCCAATTAGCACATTAGCCTTCTCTCCTTT |
| | | TGCAAGATTGCTCAGTTCATTGATGCTTAATGCTGGGCCATATC |
| | | TCTTGTCTTCTTTGCCCAAAATGAGAAATCCTCTCAGGACAGCA |
| | | GACTCCACCCCAGATGTGCCTTCATCTGGATCTTCAGTCAATGC |
| | | ACCTGCATCCTTTCCAAGAACTGTAAGTCGTTTGGTTGCCTTGT |
| | | TGTAATTGAATACTGGAGAATTGCCTCTTACCAGTATCCTCAAC |
| | | CCTGATCCTCTCACATTCACAGTCAATGAGGAAAATTGCATCCT |
| | | ACTCTGTTCTGGTGGGGCAGCAGCAAAGGGGAGAAGTTTTATT |
| | | ATTTGGACAGTGTCAAATGTCCCAAGCACATCCCGCATTTGCTG |
| | | GAACAGTGTCCTTACGAATCCACTGTACCGGCTTCTGGTTGCCT |
| | | TAGGGACAAGAGACTGAAATGGTTCAAATTCCATTTTGTTGTAT |
| | | AACATTGTGGGATCTTGTGACCATTGAATTTTCACAATTTCCCA |
| | | GTTCCTGATTATCCATTGATAAGTGTTGACTAGCACTGACTCAG |
| | | GGCCATTGATCTCCCACATCATTGATGACGAATAAGTTATTGTC |
| | | AACTTCTCAGTTCCTTGCGTTTCACTGACTTCTTCGGGAGACAA |
| | | TAGTACGTTCCCTCTTTGATCTCTAACCCTTAAAAATCGGTCAA |
| | | TACTCACTACCACTCTCTCCGTGCTGGAGTATTCATCTACTCCC |
| | | ATTTTGCTGACTCTTATCCCTCTCAGCGACATCTCCGTGCTTGGG |
| | | GTCATGTCGGGCAGTATTCCGATCATTCCCATCACATTGTCGAT |
| | | GGATTCAATTCCCCAGTTCTGGAAAAGCACTTTTGCATCTTTTT |
| | | GGAAATGCCTCAAGAGTTGGTGCATGGGGTTCAGTCGCTGGTTT |
| | | GCCCTATTGACAAAGTTCAGATCGCCCCTAACTGCCTTGATCAT |
| | | GCAATCCTCCTGTGAGAATACCATGGCCACAATTATTGCCTCAG |
| | | CAATTGACTGCTCGTCTCTCCCGCTTACTATCAACTGGATCAAT |
| | | CTCCTGGTTGCCTTTCTGAGAATAGCTGTTGCTCTTCTCCCAACC |
| | | ATTGTGAATTCTTCATACCCTTCATGTACTCTTATTTTCAGTGTT |
| | | TGGAGGTTGCCCGTTAGCACTTCTTCTTCTTTCTTGACTGATGAT |
| | | CCGCTTGTCCTTTTGAAAGTGAACCCACCAAAACTGAAAGATG |
| | | AGCTAATCCTCAACCCTATTGCTGCCTTGCATATGTCTACGGCT |
| | | TGTTCCTCAGTTGGATTCTGTCTAAGGATGTCCACCATCCTTACT |
| | | CCTCCAATCTGTGTGCTGTGGCACATTTCCAAGAGAGATGCTAA |
| | | TGGGTCTGCTGACACTGCTGCTCTTCTTACTATGTTTCTAGCAGC |
| | | GATAATCAAACTTTGGTCAACATCATCATTTCTCACTTCTCCTCC |
| | | TGGAGTGTACATCTGCTCCCAGCACGTCCCTTGGGTTAAGTGCA |
| | | ACACTTCAATATAAACACTGCCTGTTCCGCCGGCTACTGGGAGA |
| | | AACCTTGTTTTACGGACCAATTCTCTTTCTAGCATGTACGCCAC |
| | | CATCAAGGGAGCAATTTTACAATCCTGGAGCTCTTCTTTCTTCT |
| | | CTTTTGTTATTGCCAGCTGTGACTCTGATGTCAGTATTCTTGCCC |
| | | CCACTTCATTTGGGAAAACAACTTCCATAATCACATCCTGTGCC |
| | | TCCTTGGCACTGAGATCTGCATGGCCAGGGTTTGTATCAACTCT |
| | | CCTCCTTATTTTAACTTGATTTCTGAAGTGGACAGGGCCGAAGG |
| | | TACCATGTTTCAACCTTTCGACCTTTTCGAAATAAGTTTTATATA |
| | | CCTTAGGGTAATGAACTGTACTTGTTGTTGGGCCATTCCTATTC |
| | | CACCATGTTACGGCCAGAGGTGATACCATCACTCGGTCTGATCC |
| | | AGCATCGTTTGTTTTGCTCCAGAGGGTTTGTCCTTGTTCATTCCT |
| | | CTCTGGAATCATGTCCATTATTCTCTTGTCTGCTGTAATTGGGTA |
| | | TCTCATTGCCATCATCCACTTCATTCTGAGTGCGGGGTTCTTCTC |
| | | TTGCCTTCCTGATGTGTACTTTTTGATTATGGCCATATGGTCCAC |
| | | AGTGGTCTTAGTGAGTATCTCGCGAGTGCGGGACTGCGACATT |
| | | AGATCTCTCAGTTCTTTTATTCTCTCCATATTGAATATATTTGAC |
| | | CTGCTTTTGCTTAGCTTATCAGACTGATGTTGACTGTTGAATCTC |
| | | ATGGCAACAGCAGTCGATGGGCTGAGTAAAACATGGATCG |
| 3 | IVT_Seg2 | TAATACGACTCACTATAGGGATAATCTTGTTGATGCCTTAGCAG |
| | | CACGTAAATATTGGCGTTAAGATTCTAAAATTATCTCCAGTATT |
| | | AACTGTGCTGCTGAAGTAGAAACAAGGCATTTTTTCATGAAGG |
| | | ACAAGTTAAATTCATTATTTTTGCCGTCTGAGTTCTTCAATGGT |
| | | GGAACAGATCTTCATGATCTCAGAGAACTCTTCTTTCTTGATCC |
| | | GTCCAGACTCGAAGTCGACCCTGGCATCAATCCGGGCCCTAGA |
| | | CACCATGGCCTCCACCATGCTAGAAATTCCAACCGGTCTCCTAT |
| | | ATGAACTGCTAGGGAAAAATTTCTCGAATAGATTGCAGCACTT |
| | | CTGGTACATCTGTTCATCCTCAAGAATTCCCCTTTGGCTTGTGTT |
| | | GAGAATAGAACGATTCCTCTTGGGAATCCAGGAATGTGTAGTT |
| | | GCAACGGCATCATATTCCATGCTTTTGGCTGGACCATGGGCTGG |
| | | CATTACCACAGCATTGTTTACAGAATCAATCTCTTTATGACTGA |
| | | CAAAGGGATTCAGGGGATTACAAAGTCTTCCCCGATAATCATC |
| | | ATCCATTAGCTCCCATTTTAAGCAGACTTCAGGAATGTGAAGAT |
| | | TCCGTATATTGTATAAGTTTGGTCCTCCATCTGATACTAATAGC |
| | | CCTACTTTTGATTGGGTTTGATCCCACAGCTTCTTTAACTCAAAT |
| | | GATCTTCTCGTCTGAATTTGTGTGTCTCCCCTATGGCACCTATAT |
| | | GTGTATCTGTAGTCTTTGATGAACAATTGAAGAGCCATCTGGGC |
| | | CGTTGCAGGTCCAAGGTCATTGTTTATCATGTTGTTCTTTATCAC |
| | | TGTTACTCCAATACTCATGTCAGCTGATTCATTTACTCCAGACA |
| | | CTCCAAAGCTGGGTAGCTCCATGCTAAAATTAGCCACAAATCC |
| | | ATAGCGATAAAAAAAGCTTGTGAATTCAAATGTCCCTGTCTTAT |
| | | TTATATAGGACTTCTTTTTGCTCATGTTGATTCCCACTAACTTGC |
| | | AGGTCCTGTAGAATCTGTCCACTCCTGCTTGTATTCCCTCATGG |
| | | TTTGGTGCATTCACTATGAGAGCAAAATCGTCGGATGATTGGA |
| | | GCCCATCCCACCAGTATATTGTCTTGGTGTATTTCTTTTGTCCAA |
| | | GATTCAGTATCGAGACTCCCAAGACCGTACTTAGCATGTTGAAC |
| | | ATGCCCATCATCATCCCAGGACTCAGTGATGCTGTGCCATCTAT |
| | | TAGAAGAGGCCTTATTTTCTCAATTTTCTTCTTTGTTGATTCATT |
| | | GAAGTACTTCAGGTCAATGCTTGCTAGCATTTCTGCTGGTATTT |
| | | GTGTTCGAATCTTCATTCTTTTACTCTCGAACATGTACCCTTTCC |
| | | CTAGTCTTGCCATTTTGTTTGAGAACATTATGGGTGCCATGCTC |
| | | AGGATGTTTCTGAACCACTCGGGTTGATTTCTGGTGATATATGT |
| | | AATCATCGCCAGGAACATTCGAGGATTTTGATTTTCATTCCACT |
| | | TAGTGTTGTCCCCAGTGATTGTGAAAGAAATCTCTGTGTCTTGT |
| | | GAATTAGCCATCATCTTTCTCACAACATTTGCCAGTTTGGCCTT |
| | | CTTTTCATTGCCCCCTACTGGGAGCCCAGACTGTTCAAGCTTTT |
| | | CGCAAATGCTCCTAGCTAAAGTTTCAACAAAGTATACGAAACC |
| | | TCTAATCTGCATCCCAGGTGTTGCGATAGCCCTTCTTTTTAACTT |
| | | GCCTCTCTCTGCATCTTTGGTCATCGTATTTAATGTCAGTGCTCT |
| | | TATTAGATAGCCTCTCTTATTCAGTCTTTGTTTTTTCTTCCCTATT |
| | | GTTCTTTGCGTGACCATCTTCTTGGTCATGTTGTCTCTTACTCTC |
| | | CTTTTTCTTTGAAAGTGGGTTGTTATCTCTATTTCCTCTTTGTTC |
| | | ATTGATTCCATTACATCCTTTAAGAAATCTATTAGCCTTCCTGA |
| | | CTCATTAGCTGTTAGGCCATTCGATCTAAAGACTTCTATGGTGT |
| | | TGGCCAATGCAGTTGCTGCCGGTTGATTTCTGTTTAATGTCCAA |
| | | TCATAAGTCTGGCGACCTTGAGTTAGTTTATCTACCCTTGTTTGT |
| | | TGAACAACTTCCATTGTTTCAAGGCATGAATTCTCAAATATTCC |
| | | TGGGTGGGATTCTTCAAGGAAAGCCATAGCCTCTAGAACACAG |
| | | TCTGTTTGTGCATACCCACTTGGTTCATTATCCTCAGGTAGTGGT |
| | | CCATCAATCGGGTTGAGCTGGGGTGCACCAGTCTCTGTGTTTGT |
| | | CGTCCACTTTCCCTTTTCTGAGTATTGGTGTGTTCTGTTTACTGT |
| | | GTCCATGGTGTATCCTGTTCCTGTTCCATGGCTGTATGGAGGAT |
| | | CTCCAGTATAAGGGAATGTGGTGCTTATGGCATTTTGCGCTGGA |
| | | ATTTTTAGGAAAAGTAGAGTCGGATTGACATCCATTCAAATGGT |
| | | TTGCCTGCTTTTGCTTAGCTTATCAGACTGATGTTGACTGTTGAA |
| | | TCTCATGGCAACAGCAGTCGATGGGCTGAGTAAAACATGGATC |
| | | G |
| 4 | IVT_Seg3 | TAATACGACTCACTATAGGGATAATCTTGTTGATGCCTTAGCAG |
| | | CACGTAAATATTGGCGTTAAGATTCTAAAATTATCTCCAGTATT |
| | | AACTGTGCTGCTGAAGTAGAAACAAGGTACTTTTTTGGACAGT |
| | | ATGGATAGCAAATAGTAGCATTGCCACAACTACTTCAGTGCAT |
| | | GTGTGAGGAAGGAGTTGAACCAAGATGCATTAAGCAAAACCCA |
| | | GGGATCATTAATCAGGCACTCCTCGATTGCTTCATATAGCCCCC |
| | | CAAGATCGAAGGTTCCAGGTTCCAGGTTGTCCCTAAGTGCCTGA |
| | | ACAATGAGAAGCAATTTTCTAGATTCAGCCGAAAACCCCTCAA |
| | | GTTGTGGAGACGCATATAGACTGTTGAATACAGATTTTGCCAGT |
| | | AAGGTCCTGCACACTTTCCCAATAGAGCCTTCCTCCACTCCCCT |
| | | GGGTGATTCTCCGATTGGCCATGTTTCCGATTTGTTTTCAAAGA |
| | | ATTCCTTGGTCATGTCTTTCTCTTTGACAGAAGACTCGGCCTCA |
| | | ATCATGCTCTCAATCTGCTGAAGAGACTGAAGAAGGCAGCGCC |
| | | TCATTTCCATGCCCCATTTCATCTTGATCTTGGAGGTTCCATTGG |
| | | TTCTCACATATAGGAACATGGGCCTCGACACTTGGCCTATCGCA |
| | | GTCCTCAAGAGCATGTCTCCTATTTCAAGAACACAGTATTTTTC |
| | | CCATTTGTGTGGCTCCAGTCTCGGGTCAGTGAGTGAGAACTCCA |
| | | TACTTACAAAGTTCACCACATCAGTATCATTTCTCAAATGAGAC |
| | | CTTCCTTTTATAATGAACCCATACAGGTTTGTTTTCCGTCTTCCT |
| | | TCTTTGGTCCTACATTTGCTTATCATTGGGATCAGCTGAAAGTC |
| | | ATCCATGGCTGCACAGGATGCATTGAGCAAGGCCGTATTTATGT |
| | | ACACTCCCTTCATTATGTATTCAGTAGCCCTGCAGTGGGACACT |
| | | TCTGCTGTAAAATAGTTCCTCCTCATGCTTGCGATATGTTCAAT |
| | | CGGGGCAACATCTTCTCCTATTTCATCAAGTTCTATCCAGCTTG |
| | | AATCAGTCAATTCACATGCCTTATTGAATTCATTTTGGACCCAG |
| | | CTTGCTAGAGATCTGGGCTCTGGCTCATCACTGTCATACTGTTT |
| | | AAGGTCTCCAACATCTTTGCAGTCATCAAAGTCTACTTTTTCTG |
| | | GTGCCATATTTTCACCGAGTGCCCACTTCAATTGGCTTGTTCTCT |
| | | TCATGTTCTTTGTCCTTGGGATCTTCTCTTCATTTTCAATGTCCT |
| | | GTAGCTCTGCTAGCACCTGCTTCCAAGCCATGAGGTAATTGGGA |
| | | TTTATGCCTTTCTCATGTGGTTTGACTATGTTAGGCTCTTTCCAG |
| | | CCAAAGAATGTCTTCATGCATTTGATTGCATCATATAGTGGTAT |
| | | TCCCTCCCCCTCGTGACTCGGGTCTTCAATACTTAATTTCAGAG |
| | | CATCCATCAGCAGGAACTTTGACCGCTGATGGCAAAGAGGCCC |
| | | ATCAGGCAATCTGAGGGGGCGTGGTGTCGTCCTCAAGAATGGT |
| | | TCAATTTTGGCGTTCACTTCTTTTGACATTTGGGAAAGCTTGCCC |
| | | TCAATGCAGCCGTTCGGCTCGAATCCATCTACATAGGCTCTAAA |
| | | GTTTTCAAGGCTGGGGAAGTTCGGTGGGAGACTTTGGTCGGCA |
| | | AGCTTGCGCATAGTTCCTGTAATCTCGAATTTTTCTTCAATTGTC |
| | | TCTTCGCCTCTTTCGGACTGACGAAAGGAATCCCATAGACTCCT |
| | | ACTGGCCATTTCTTGTCTTATAGTGAAAAGCCTAGTTTTGATTCT |
| | | TGCCCTGCTCTCTTCGTCAAGGGTGTAGTCCGCTTTGGTGGCCA |
| | | TCTCCTCTCCAGTGAATGAAAAGATGTGAATGTGTGTCTTCTCA |
| | | GATTTTATTTTGTTGGCTTTCTCTAGGTAATATATGTGGACTTCC |
| | | CTCCGTGTTACTCCAATTTCAATGAACCGGTTCTCTTTGTAATCA |
| | | TACAAATCAGGAAGAAATTTAGGCTTCTCTACCCCTGTTGTGTT |
| | | ACATATACTGTTCACCACTGTCCAGGCCATGATTCGGTCTCTTC |
| | | CTTCAATTATCTCAAATCGGTGCTTCAATAGTGCATTCGGGTCA |
| | | CCAGATTCTACAATTATTGATTCACCCCGTTCGTCGATGAAATG |
| | | GAAATCCGAATACATGAAACAAACTTCCAAATGTGTGCATATT |
| | | GCAGCAAACTTGTTAGTTTCGATTTTCGGATCTTCCCCATATTCT |
| | | TTCATTGCCTTTTCCGCAAGCTCGACGATCATTGGATTGAAGCA |
| | | TTGTCGCACAAAGTCTTCCATTTTGGATCAGTACCTGCTTTTGCT |
| | | TAGCTTATCAGACTGATGTTGACTGTTGAATCTCATGGCAACAG |
| | | CAGTCGATGGGCTGAGTAAAACATGGATCG |
| 5 | IVT_Seg4 | TAATACGACTCACTATAGGGATAATCTTGTTGATGCCTTAGCAG |
| | | CACGTAAATATTGGCGTTAAGATTCTAAAATTATCTCCAGTATT |
| | | AACTGTGCTGCTGAAGTAGAAACAAGGGTGTTTTTCTCATGCTT |
| | | CTGAAATCCTAATGTTAAATACATATTCTACACTGTAGAGACCC |
| | | ATTAGAGCACATCCAGAAACTGATTGCCCCCAGGGAGACTACC |
| | | AGTACCAATGAACTGGCGACAGTTGAATAGATCGCCAAAATCT |
| | | GGTAAATCCTTGTTGATTCCAGCTTTACCCCATCTATTTCTTCTC |
| | | TGTTTAATTTTGCTTCCTCTGAGTATTTTGGGTAGTCATAAGTCC |
| | | CATTTTTGACACTTTCCATGCACGTGTTATCGCATTTGTGGTAA |
| | | AATTCAAAGCAGCCGTTTCCAATTTCCTTGGCATTGTTTTTTAGC |
| | | TGGCTTCTTACCTTTTCATATAAGTTCTTCACATTTGAATCGTGG |
| | | TAGTCCAAAGTTCTTTCATTTTCCAATAGAACCAACAGTTCGGC |
| | | ATTGTAAGTCCAAATGTCCAGGAAACCATCATCAACTTTTTTAT |
| | | TTAAATTCTCTACTCTTTTTTCCAGGTGGTTGAACTCTTTACCTA |
| | | CTGCTGTGAACTGTGTATTCATCTTTTCAATAACAGAATTTACTT |
| | | TGTTAGTAATCTCGTCAATGGCATTCTGTGTGCTCTTCAGGTCG |
| | | GCTGCATATCCTGACCCCTGCTCATTTTGATGGTGATAACCGTA |
| | | CCATCCATCTACCATCCCTGTCCACCCCCCTTCAATGAAACCGG |
| | | CAATGGCCCCAAATAGGCCTCTAGATTGAATAGACGGGATATT |
| | | CCTCAATCCTGTGGCCAGTCTCAATTTTGTGCTTTTTACATATTT |
| | | TGGACATTTTCCAATTGTGATCGGATGTATATTCTGAAATGGGA |
| | | GGCTGGTGTTTATAGCACCCTTGGGTGTTTGACAAGTTGTATTG |
| | | CAATCGTGGACTGGTGTATCTGAAATGATAATACCAGATCCAG |
| | | CATTTCTTTCCATTGCGAATGCATATCTCGGTACCACTAGATTTC |
| | | CAGTTGCTTCGAATGTTATTTTGTCTCCCGGCTCTACTAGTGTCC |
| | | AGTAATAGTTCATTCTCCCTTCTTGATCCCTCACTTTGGGTCTTA |
| | | TTGCTATTTCCGGCTTGAACTTCTTGCTGTATCTTGATGACCCCA |
| | | CAAAAACATATGTATCTGCATTCTGATAGAGACTTTGTTGGTCA |
| | | GCACTAGTAGATGGATGGTGAATGCCCCATAGCACGAGGACTT |
| | | CTTTCCCTTTATCATTAATGTAGGATTTGCTGAGCTTTGGGTATG |
| | | AATTTCCTTTTTTAACTAGCCATATTAAATTTTTGTAGAAGCTTT |
| | | TTGCTCCAGCATGAGGACATGCTGCCGTTACACCTTTGTTCGAG |
| | | TCATGATTGGGCCATGAACTTGTCTTGGGGAATATCTCAAACCT |
| | | TTCAAATGATGACACTGAGCTCAATTGCTCTCTTAGCTCCTCAT |
| | | AATCGATGAAATCTCCTGGGTAACACGTTCCATTGTCTGAACTA |
| | | GGTGTTTCCACAATGTAGGACCATGAGCTTGCTGTGGAGAGTG |
| | | ATTCACACTCTGGATTTCCCAGGATCCAGCCAGCAATGTTACAT |
| | | TTACCCAAATGCAATGGGGCTACCCCTCTTAGTTTGCATAGTTT |
| | | CCCGTTATGCTTGTCTTCTAGAAGGTTAACAGAGTGTGTTACTG |
| | | TTACATTCTTTTCTAGTACTGTGTCTACAGTGTCTGTTGAATTGT |
| | | TCGCATGATAACCTATACATAATGTGTCTGCATTTGCGGTTGCA |
| | | AATGTATATAGCAGAACTACTAGTACCTGCTTTTGCTTAGCTTA |
| | | TCAGACTGATGTTGACTGTTGAATCTCATGGCAACAGCAGTCGA |
| | | TGGGCTGAGTAAAACATGGATCG |
| 6 | IVT_Seg5 | TAATACGACTCACTATAGGGATAATCTTGTTGATGCCTTAGCAG |
| | | CACGTAAATATTGGCGTTAAGATTCTAAAATTATCTCCAGTATT |
| | | AACTGTGCTGCTGAAGTAGAAACAAGGGTATTTTTCCTCAACTG |
| | | TCATACTCCTCTGCATTGTCTCCGAAGAAATAAGACCCTTCATT |
| | | ACTCATGTCAAAGGAAGGCACGATCGGGTTCGTTGCCTTTTCGT |
| | | CCGAGAGCTCGAAGACTCCCCGCCCCTGGAAGGACAAATCTTC |
| | | TGGCTTTGCACTTTCCATCATTCTTATAACTTCTGTTCGCATGTC |
| | | GGATGTCCGTCCTTCATTGTTCCCGCTGAATGCTGCCATAACGG |
| | | TTGCTCTTTCAAAAGGGAGATTCCGCTGCACTGAGAATGTAGGC |
| | | TGCACACTGATCTGGCCTGCGGATGCCTTTTGTTGATTGGTATT |
| | | TCCTCCACTCCTGGTCCTTATGGCCCAGTATCTGCTTCTCAGTTC |
| | | CAGGGTATTGGAGTTCATGGTTTCCACATTCTCATTTGAAGCAA |
| | | TCTGGACCCCTCTTGTGGAAAGCTTTCCTCTTGGAATCACTTTCT |
| | | TTCCTCTTATGAAACTTGATACTCTTAAATCTTCAAATGCAGCA |
| | | GAGTGGCATGCCATCCACACCAATTGACTCTTGTGAGCTGGGTT |
| | | TTCATTTGGTCTCATCAGGCTGACCACTTGGCTGTTTTGGAGTA |
| | | ATTTGAATGGGTCTATCCCGACCAGTGAGTACCCTTCCCTTTCA |
| | | AAGTCATGCCCACTTGCTACTGCAAGCCCATACACACAAGCAG |
| | | GCAGGCAGGATTTATGTGCAACTGATCCCCTCAGAATGAGTGC |
| | | TGACCGTGCCAGGAAAATGAGGTCTTCAATCTCAGCGTTTCCTG |
| | | GGTTTCGACTTTCTCTTACTTGATCCATCATTGCCCTCTGGGCAG |
| | | CTGTTTGAAATTTTCCTTTGAGGATATTGCACATTCTTTCATAAG |
| | | CAACCCTTGTCCTTCGTCCATTTTCACCCCTCCAGAAATTTCGGT |
| | | CATTGATTCCACGTTTGATCATTCTGATTAACTCCATTGCTATTG |
| | | TTCCAACTCCTTTCACCGCAGCACCTGCGGCACCAGACCTTCTG |
| | | GGAAGTGTTGAACCTTGCATTAGAGAGCACATTCTGGGATCCA |
| | | TTCCGGTGCGAACAAGCGCTCTTGTTCTCTGATATGTGGCATCA |
| | | TTCAGGTTGGAATGCCAAATCATGATATGAGTAAGACCTGCTGT |
| | | TGCATCTTCGCCATTGTTTGCTTGGCGCCAAACTCTCCTTATTTC |
| | | TTCTTTGTCATAAAGGATGAGTTCTCTCATCCACTTTCCGTCTAC |
| | | TCTTCTATATATGGGTCCTCCTGTTTTCTTAGGGTCCTTCCCAGC |
| | | ACTGGGATGCTCTTCTAGGTATTTATTTCTTCTCTCATCAAAAGC |
| | | AGAAAGCACCATCCTCTCTATTGTTATGCTATTCTGGATTAGTC |
| | | GTCCATCATAATCACTGAGTTTGAGTTCAGTGCACATTTGGATG |
| | | TAGAATCTCCCGATTCCACCAATCATTCTTCCGACAGATGCTCT |
| | | GATTTCTGTGGCATCCTGGCGCTCCCCACCAGTCTCCATTTGTTC |
| | | ATATGATCGTTTGGTGCCTTGAGACGCCATGGCTTCGATGTCAC |
| | | TCATTGAGTGATTATCTACCCTGCTTTTGCTTAGCTTATCAGACT |
| | | GATGTTGACTGTTGAATCTCATGGCAACAGCAGTCGATGGGCT |
| | | GAGTAAAACATGGATCG |
| 7 | IVT_Seg6 | TAATACGACTCACTATAGGGATAATCTTGTTGATGCCTTAGCAG |
| | | CACGTAAATATTGGCGTTAAGATTCTAAAATTATCTCCAGTATT |
| | | AACTGTGCTGCTGAAGTAGAAACAAGGAGTTTTTTGAACAAAT |
| | | TACTTGTCAATGGTAAATGGCAACTCAGCACCGTCTGGCCAAG |
| | | ACCAACCCACAGTGTCACTGTTTACACCACAAAAGGATATGCT |
| | | GCTCCCGCTAGTCCAGATTGTGTTCTCTTTGGGTCGCCCTCTGAT |
| | | TAGTTCAACCCAGAAGCAAGGTCTTATACAATCCAGCCCTGTTA |
| | | GTTCTGGATGCTGAACAAAACTCCCGCTATATCCTGACCACTCA |
| | | TTTATTCCTACGATATCTTGCTTTATTGAGAAGTTATTGTCTGTC |
| | | CCAGTCCATCCGTTCGGATCCCAAATCATCTCAAAACCGTTTCT |
| | | TGAACTAATGCTTTTAGTTCTCCCTATCCAAACACCATTGCCGT |
| | | ATTTGAATGAAAACCCTTTTACTCCATTTGCTCCATTAGACGAT |
| | | ACTGGACCACAACTGCCTGTCTTATCATTAGGGCGTGGATTGTC |
| | | TCCGAAAATCCCACTGCATATGTATCCTATCTGATATTCCAGAT |
| | | TCTGGTTGAAAGACACCCACGGTCGATTCGAGCCATGCCAGTT |
| | | ATCCCTGCACACACATGTGATTTCACTAGAATCAGGATAACAG |
| | | GAGCATTCCTCATAGTGATAATTAGGGGCATTCATTTCGACTGA |
| | | TTTGACTATCTTTCCCTTTTCTATTCTGAAGATCTTGTATGAGGC |
| | | CTGTCCATTACTTGGTCCATCGGTCATTACAGTAAAGCAAGAAC |
| | | CATTTACACATGCACATTCAGACTCTTGTGTTCTCAATATATTGT |
| | | TTCTCCAACTCTTGATAGTGTCTGTTATTATGCCGTTGTACTTTA |
| | | ACACAGCCACTGCCCCATTGTCTGGGCCAGAAATTCCAATTGTT |
| | | AGCCAATTGATGCCATCATGACAAGCACTTGCTGACCAAGCGA |
| | | CTGACTCAAATCTTGAGTTGTATGGAGAGGGAACTTCACCAAT |
| | | AGGACAGCTCATTAGGGTTCGATATGGGCTCCTGTCTTTAATGG |
| | | TTCCATTGGAATGTTTGTCATTTAGCAAGGCCCCTTGAGTCAAG |
| | | AAGAAGGTTCTGCATTCCAAGGGGGAGCATGATATGAATGGTT |
| | | CCCTTATGACAAACACATCCCCCTTGGAACCGATTCTTACACTG |
| | | TTGTCTTTACTGTATATAGCCCATCCACTAACAGGGCAGAGAGA |
| | | GGAATTGCCCGCTAATTTCACGGAAACCACTGACTGTCCAGCA |
| | | GCAAAGTTGGTGTTGCTGATGTTAACATATGTCTGATTTACCCA |
| | | AGTGTTGTTTTCATAAGTAATGACGCTTTGATTGCATGTTTCAA |
| | | TCTGATTTTGATTCCCAAGTTGAATTGAGTGGCTAATCCATATT |
| | | GAGATTATGTTTCCAATTTGTAATATTAAGTTAGCCATTCCAAT |
| | | TGTCATACAGACCGAACCAATGGTTATTATCTTTTGGTTTGGAT |
| | | TCATTTTAAACCCCTGCTTTTGCTCCTGCTTTTGCTTAGCTTATC |
| | | AGACTGATGTTGACTGTTGAATCTCATGGCAACAGCAGTCGAT |
| | | GGGCTGAGTAAAACATGGATCG |
| 8 | IVT_Seg7 | TAATACGACTCACTATAGGGATAATCTTGTTGATGCCTTAGCAG |
| | | CACGTAAATATTGGCGTTAAGATTCTAAAATTATCTCCAGTATT |
| | | AACTGTGCTGCTGAAGTAGAAACAAGGTAGTTTTTTACTCTAGC |
| | | TCTATGTTGACAAAATGACCATCGTCAACATCCACAGCACTCTG |
| | | CTGTTCCTGTTGATATTCTTCCCTCATGGACTCAGGCACTCCTTC |
| | | CGTAGAAGGCCCTCTTTTCAAACCGTATTTAAAGCGACGATAA |
| | | ATACATTTGAAAAAAAGACGATCAGTAATCCACAATATCAGGT |
| | | GCAAGATCCCAATGATATTTGCTGCAATGACGAGAGGATCACT |
| | | TGAATCGCTGCATCTGCACTCCCATTCGCTTCTGGTAGGCCTGC |
| | | AAATTTTCAAGAAGGTCATCTTTCAGACCAGCACTGGAGCTAG |
| | | GATGAGTCCCAATAGTTCTCATTGCATGTACCATCTGCCTAGTC |
| | | TGATTAGCAACCTCCATGGCCTCCGCTGCCTGTTCACTCGATCC |
| | | AGCCATCTGTTCCATAGCCTTTGCCGTAGTGCTAGCCAGCACCA |
| | | TTCTGTTTTCATGCCTGATTAGTGGATTGGTGGTAGTAGCCATC |
| | | TGTCTGTGAGACCGATGCTGTGAATCAGCAATCTGTTCACAAGT |
| | | GGCACACACTAGACCAAAAGCAGCTTCTGTGGTCACTGTTCCC |
| | | ATCCTGTTGTATATGAGGCCCATGCAACTGGCAAGTGCACCAGT |
| | | TGAATAGCTTAGTGACACCTCCTTGGCCCCATGGAACGTTATTT |
| | | CTCTTTTGAGCTTCTTGTATAGTTTAACTGCTCTATCCATGTTGT |
| | | TCGGGTCCCCATTCCCATTTAGGGCATTTTGGACAAAGCGTCTA |
| | | CGCTGCAGTCCTCGCTCACTGGGCACGGTGAGCGTGAACACAA |
| | | ATCCTAAAATTCCCTTAGTCAGAGGTGACAAGATTGGTCTTGTC |
| | | TTTAGCCATTCCATGAGAGCCTCAAGATCTGTGTTCTTTCCTGC |
| | | AAAGACACTTTCCAGTTTCTGCGCGATCTCGGCTTTGAGGGGGC |
| | | CTGACGGGATGATAGAAAGAACGTACGTTTCGACCTCGGTTAG |
| | | AAGACTCATCTTTAAATATCTACCTGCTTTTGCTTAGCTTATCAG |
| | | ACTGATGTTGACTGTTGAATCTCATGGCAACAGCAGTCGATGG |
| | | GCTGAGTAAAACATGGATCG |
| 9 | IVT_Seg8 | TAATACGACTCACTATAGGGATAATCTTGTTGATGCCTTAGCAG |
| | | CACGTAAATATTGGCGTTAAGATTCTAAAATTATCTCCAGTATT |
| | | AACTGTGCTGCTGAAGTAGAAACAAGGGTGTTTTTTATCATTAA |
| | | ATAAGCTGAAACGAGAAAGCTCTTATCTCTTGTTCTACTTCAAG |
| | | CAGTAGTTGTAAGGCTTGCATAAATGTTATTTGTTCGAAACTAT |
| | | TCTCTGTCGCTTTCAATCTGTGCCGCATTTCTTCAATTAACCACC |
| | | TTATTTCCTCAAATTTCTGTCCCAATTGCTCTCGCCACTTTTCAT |
| | | TTCTGCTCTGGAGGTAGTGAAGGTCTCCCATTCTCATCACAGTT |
| | | TCTCCAAGCGAATCTCTGTATATTTTCAGAGACTCGAACCGTGT |
| | | TACCATTCCATTCAAGTCCTCCGATGAGGACCCCAACTGCATTT |
| | | TTGACATCCTCATAAGTATGTCCTGGAAGAGAAGGTAATGGTG |
| | | AAATTTCTCCAACTATTGCTCCCTCCTCAGTGAAAGCCCTTAGT |
| | | AGTATCAAGGTCTCTAATCGGTTAAAGATTACACTGAAGTTCGC |
| | | TTTCAGTACTATGTTCTTTTCCATGATCGCCTGGTCCAATCGCAC |
| | | GCAAAGAGGGCCTATTATCTTTTGCCTAGGCATGAGCATGAAC |
| | | CAGTCTCGTGACATTTCATCGAGGGTCATGTCAGAAAGGTACC |
| | | GCGAAGTAGGTACAGATGCAATTGTCATTCTAAGTGTCTCGCTG |
| | | GATTCCTCTTTCAAGATCCATTCCACGATTTGTTTCCCAACAAG |
| | | AGTGGCTGTTTCGATATCGAGGCCAAGGGTGTTGCCTCTTCCTT |
| | | TTAAGGACTTTTGATCTCGGCGGAGCCGATCAAGGAATGGGGC |
| | | ATCACCCAATCCATTGTCTGCAAATCGCTTGCGGATATGCCAAA |
| | | GGAAACAGTCTACCTGAAAGCTTGACATGGTGTTGGAGTCCAT |
| | | TATGTTTTTGTCACCCTGCTTTTGCTTAGCTTATCAGACTGATGT |
| | | TGACTGTTGAATCTCATGGCAACAGCAGTCGATGGGCTGAGTA |
| | | AAACATGGATCG |
| 10 | IVT_PB2 | TAATACGACTCACTATAGGGATAATACCATGGAACGCATTAAA |
| | | GAACTGCGCGATCTGATGAGCCAGAGCCGCACCCGCGAAATTC |
| | | TGACCAAAACCACCGTGGATCATATGGCGATTATTAAAAAATA |
| | | TACCAGCGGCCGCCAGGAAAAAAACCCGGCGCTGCGCATGAA |
| | | ATGGATGATGGCGATGCGCTATCCGATTACCGCGGATAAACGC |
| | | ATTATGGATATGATTCCGGAACGCAACGAACAGGGCCAGACCC |
| | | TGTGGAGCAAAACCAACGATGCGGGCAGCGATCGCGTGATGGT |
| | | GAGCCCGCTGGCGGTGACCTGGTGGAACCGCAACGGCCCGACC |
| | | ACCAGCACCGTGCATTATCCGAAAGTGTATAAAACCTATTTTGA |
| | | AAAAGTGGAACGCCTGAAACATGGCACCTTTGGCCCGGTGCAT |
| | | TTTCGCAACCAGGTGAAAATTCGCCGCCGCGTGGATACCAACC |
| | | CGGGCCATGCGGATCTGAGCGCGAAAGAAGCGCAGGATGTGAT |
| | | TATGGAAGTGGTGTTTCCGAACGAAGTGGGCGCGCGCATTCTG |
| | | ACCAGCGAAAGCCAGCTGGCGATTACCAAAGAAAAAAAAGAA |
| | | GAACTGCAGGATTGCAAAATTGCGCCGCTGATGGTGGCGTATA |
| | | TGCTGGAACGCGAACTGGTGCGCAAAACCCGCTTTCTGCCGGT |
| | | GGCGGGCGGCACCGGCAGCGTGTATATTGAAGTGCTGCATCTG |
| | | ACCCAGGGCACCTGCTGGGAACAGATGTATACCCCGGGCGGCG |
| | | AAGTGCGCAACGATGATGTGGATCAGAGCCTGATTATTGCGGC |
| | | GCGCAACATTGTGCGCCGCGCGGCGGTGAGCGCGGATCCGCTG |
| | | GCGAGCCTGCTGGAAATGTGCCATAGCACCCAGATTGGCGGCG |
| | | TGCGCATGGTGGATATTCTGCGCCAGAACCCGACCGAAGAACA |
| | | GGCGGTGGATATTTGCAAAGCGGCGATTGGCCTGCGCATTAGC |
| | | AGCAGCTTTAGCTTTGGCGGCTTTACCTTTAAACGCACCAGCGG |
| | | CAGCAGCGTGAAAAAAGAAGAAGAAGTGCTGACCGGCAACCT |
| | | GCAGACCCTGAAAATTCGCGTGCATGAAGGCTATGAAGAATTT |
| | | ACCATGGTGGGCCGCCGCGCGACCGCGATTCTGCGCAAAGCGA |
| | | CCCGCCGCCTGATTCAGCTGATTGTGAGCGGCCGCGATGAACA |
| | | GAGCATTGCGGAAGCGATTATTGTGGCGATGGTGTTTAGCCAG |
| | | GAAGATTGCATGATTAAAGCGGTGCGCGGCGATCTGAACTTTG |
| | | TGAACCGCGCGAACCAGCGCCTGAACCCGATGCATCAGCTGCT |
| | | GCGCCATTTTCAGAAAGATGCGAAAGTGCTGTTTCAGAACTGG |
| | | GGCATTGAAAGCATTGATAACGTGATGGGCATGATTGGCATTC |
| | | TGCCGGATATGACCCCGAGCACCGAAATGAGCCTGCGCGGCAT |
| | | TCGCGTGAGCAAAATGGGCGTGGATGAATATAGCAGCACCGAA |
| | | CGCGTGGTGGTGAGCATTGATCGCTTTCTGCGCGTGCGCGATCA |
| | | GCGCGGCAACGTGCTGCTGAGCCCGGAAGAAGTGAGCGAAAC |
| | | CCAGGGCACCGAAAAACTGACCATTACCTATAGCAGCAGCATG |
| | | ATGTGGGAAATTAACGGCCCGGAAAGCGTGCTGGTGAACACCT |
| | | ATCAGTGGATTATTCGCAACTGGGAAATTGTGAAAATTCAGTG |
| | | GAGCCAGGATCCGACCATGCTGTATAACAAAATGGAATTTGAA |
| | | CCGTTTCAGAGCCTGGTGCCGAAAGCGACCCGCAGCCGCTATA |
| | | GCGGCTTTGTGCGCACCCTGTTTCAGCAGATGCGCGATGTGCTG |
| | | GGCACCTTTGATACCGTGCAGATTATTAAACTGCTGCCGTTTGC |
| | | GGCGGCGCCGCCGGAACAGAGCCGCATGCAGTTTAGCAGCCTG |
| | | ACCGTGAACGTGCGCGGCAGCGGCCTGCGCATTCTGGTGCGCG |
| | | GCAACAGCCCGGTGTTTAACTATAACAAAGCGACCAAACGCCT |
| | | GACCGTGCTGGGCAAAGATGCGGGCGCGCTGACCGAAGATCCG |
| | | GATGAAGGCACCAGCGGCGTGGAAAGCGCGGTGCTGCGCGGCT |
| | | TTCTGATTCTGGGCAAAGAAGATAAACGCTATGGCCCGGCGCT |
| | | GAGCATTAACGAACTGAGCAACCTGGCGAAAGGCGAAAAAGC |
| | | GAACGTGCTGATTGGCCAGGGCGATGTGGTGCTGGTGATGAAA |
| | | CGCAAACGCGATAGCAGCATTCTGACCGATAGCCAGACCGCGA |
| | | CCAAACGCATTCGCATGGCGATTAACTAGATCG |
| 11 | IVT_PB1 | TAATACGACTCACTATAGGGATAATACCATGGATGTGAACCCG |
| | | ACCCTGCTGTTTCTGAAAATTCCGGCGCAGAACGCGATTAGCAC |
| | | CACCTTTCCGTATACCGGCGATCCGCCGTATAGCCATGGCACCG |
| | | GCACCGGCTATACCATGGATACCGTGAACCGCACCCATCAGTA |
| | | TAGCGAAAAAGGCAAATGGACCACCAACACCGAAACCGGCGC |
| | | GCCGCAGCTGAACCCGATTGATGGCCCGCTGCCGGAAGATAAC |
| | | GAACCGAGCGGCTATGCGCAGACCGATTGCGTGCTGGAAGCGA |
| | | TGGCGTTTCTGGAAGAAAGCCATCCGGGCATTTTTGAAAACAG |
| | | CTGCCTGGAAACCATGGAAGTGGTGCAGCAGACCCGCGTGGAT |
| | | AAACTGACCCAGGGCCGCCAGACCTATGATTGGACCCTGAACC |
| | | GCAACCAGCCGGCGGCGACCGCGCTGGCGAACACCATTGAAGT |
| | | GTTTCGCAGCAACGGCCTGACCGCGAACGAAAGCGGCCGCCTG |
| | | ATTGATTTTCTGAAAGATGTGATGGAAAGCATGAACAAAGAAG |
| | | AAATTGAAATTACCACCCATTTTCAGCGCAAACGCCGCGTGCG |
| | | CGATAACATGACCAAAAAAATGGTGACCCAGCGCACCATTGGC |
| | | AAAAAAAAACAGCGCCTGAACAAACGCGGCTATCTGATTCGCG |
| | | CGCTGACCCTGAACACCATGACCAAAGATGCGGAACGCGGCAA |
| | | ACTGAAACGCCGCGCGATTGCGACCCCGGGCATGCAGATTCGC |
| | | GGCTTTGTGTATTTTGTGGAAACCCTGGCGCGCAGCATTTGCGA |
| | | AAAACTGGAACAGAGCGGCCTGCCGGTGGGCGGCAACGAAAA |
| | | AAAAGCGAAACTGGCGAACGTGGTGCGCAAAATGATGGCGAA |
| | | CAGCCAGGATACCGAAATTAGCTTTACCATTACCGGCGATAAC |
| | | ACCAAATGGAACGAAAACCAGAACCCGCGCATGTTTCTGGCGA |
| | | TGATTACCTATATTACCCGCAACCAGCCGGAATGGTTTCGCAAC |
| | | ATTCTGAGCATGGCGCCGATTATGTTTAGCAACAAAATGGCGC |
| | | GCCTGGGCAAAGGCTATATGTTTGAAAGCAAACGCATGAAAAT |
| | | TCGCACCCAGATTCCGGCGGAAATGCTGGCGAGCATTGATCTG |
| | | AAATATTTTAACGAAAGCACCAAAAAAAAAATTGAAAAAATTC |
| | | GCCCGCTGCTGATTGATGGCACCGCGAGCCTGAGCCCGGGCAT |
| | | GATGATGGGCATGTTTAACATGCTGAGCACCGTGCTGGGCGTG |
| | | AGCATTCTGAACCTGGGCCAGAAAAAATATACCAAAACCATTT |
| | | ATTGGTGGGATGGCCTGCAGAGCAGCGATGATTTTGCGCTGATT |
| | | GTGAACGCGCCGAACCATGAAGGCATTCAGGCGGGCGTGGATC |
| | | GCTTTTATCGCACCTGCAAACTGGTGGGCATTAACATGAGCAA |
| | | AAAAAAAAGCTATATTAACAAAACCGGCACCTTTGAATTTACC |
| | | AGCTTTTTTTATCGCTATGGCTTTGTGGCGAACTTTAGCATGGA |
| | | ACTGCCGAGCTTTGGCGTGAGCGGCGTGAACGAAAGCGCGGAT |
| | | ATGAGCATTGGCGTGACCGTGATTAAAAACAACATGATTAACA |
| | | ACGATCTGGGCCCGGCGACCGCGCAGATGGCGCTGCAGCTGTT |
| | | TATTAAAGATTATCGCTATACCTATCGCTGCCATCGCGGCGATA |
| | | CCCAGATTCAGACCCGCCGCAGCTTTGAACTGAAAAAACTGTG |
| | | GGATCAGACCCAGAGCAAAGTGGGCCTGCTGGTGAGCGATGGC |
| | | GGCCCGAACCTGTATAACATTCGCAACCTGCATATTCCGGAAGT |
| | | GTGCCTGAAATGGGAACTGATGGATGATGATTATCGCGGCCGC |
| | | CTGTGCAACCCGCTGAACCCGTTTGTGAGCCATAAAGAAATTG |
| | | ATAGCGTGAACAACGCGGTGGTGATGCCGGCGCATGGCCCGGC |
| | | GAAAAGCATGGAATATGATGCGGTGGCGACCACCCATAGCTGG |
| | | ATTCCGAAACGCAACCGCAGCATTCTGAACACCAGCCAGCGCG |
| | | GCATTCTGGAAGATGAACAGATGTATCAGAAATGCTGCAACCT |
| | | GTTTGAAAAATTTTTTCCGAGCAGCAGCTATCGCCGCCCGGTGG |
| | | GCATTAGCAGCATGGTGGAAGCGATGGTGAGCCGCGCGCGCAT |
| | | TGATGCGCGCGTGGATTTTGAAAGCGGCCGCATTAAAAAAGAA |
| | | GAATTTAGCGAAATTATGAAAATTTGCAGCACCATTGAAGAAC |
| | | TGCGCCGCCAGAAATAGATCG |
| 12 | IVT_PA | TAATACGACTCACTATAGGGATAATACCATGGAAGATTTTGTGC |
| | | GCCAGTGCTTTAACCCGATGATTGTGGAACTGGCGGAAAAAGC |
| | | GATGAAAGAATATGGCGAAGATCCGAAAATTGAAACCAACAA |
| | | ATTTGCGGCGATTTGCACCCATCTGGAAGTGTGCTTTATGTATA |
| | | GCGATTTTCATTTTATTGATGAACGCGGCGAAAGCATTATTGTG |
| | | GAAAGCGGCGATCCGAACGCGCTGCTGAAACATCGCTTTGAAA |
| | | TTATTGAAGGCCGCGATCGCATTATGGCGTGGACCGTGGTGAA |
| | | CAGCATTTGCAACACCACCGGCGTGGAAAAACCGAAATTTCTG |
| | | CCGGATCTGTATGATTATAAAGAAAACCGCTTTATTGAAATTGG |
| | | CGTGACCCGCCGCGAAGTGCATATTTATTATCTGGAAAAAGCG |
| | | AACAAAATTAAAAGCGAAAAAACCCATATTCATATTTTTAGCTT |
| | | TACCGGCGAAGAAATGGCGACCAAAGCGGATTATACCCTGGAT |
| | | GAAGAAAGCCGCGCGCGCATTAAAACCCGCCTGTTTACCATTC |
| | | GCCAGGAAATGGCGAGCCGCAGCCTGTGGGATAGCTTTCGCCA |
| | | GAGCGAACGCGGCGAAGAAACCATTGAAGAAAAATTTGAAATT |
| | | ACCGGCACCATGCGCAAACTGGCGGATCAGAGCCTGCCGCCGA |
| | | ACTTTCCGAGCCTGGAAAACTTTCGCGCGTATGTGGATGGCTTT |
| | | GAACCGAACGGCTGCATTGAAGGCAAACTGAGCCAGATGAGCA |
| | | AAGAAGTGAACGCGAAAATTGAACCGTTTCTGCGCACCACCCC |
| | | GCGCCCGCTGCGCCTGCCGGATGGCCCGCTGTGCCATCAGCGC |
| | | AGCAAATTTCTGCTGATGGATGCGCTGAAACTGAGCATTGAAG |
| | | ATCCGAGCCATGAAGGCGAAGGCATTCCGCTGTATGATGCGAT |
| | | TAAATGCATGAAAACCTTTTTTGGCTGGAAAGAACCGAACATT |
| | | GTGAAACCGCATGAAAAAGGCATTAACCCGAACTATCTGATGG |
| | | CGTGGAAACAGGTGCTGGCGGAACTGCAGGATATTGAAAACGA |
| | | AGAAAAAATTCCGCGCACCAAAAACATGAAACGCACCAGCCA |
| | | GCTGAAATGGGCGCTGGGCGAAAACATGGCGCCGGAAAAAGT |
| | | GGATTTTGATGATTGCAAAGATGTGGGCGATCTGAAACAGTAT |
| | | GATAGCGATGAACCGGAACCGCGCAGCCTGGCGAGCTGGGTGC |
| | | AGAACGAATTTAACAAAGCGTGCGAACTGACCGATAGCAGCTG |
| | | GATTGAACTGGATGAAATTGGCGAAGATGTGGCGCCGATTGAA |
| | | CATATTGCGAGCATGCGCCGCAACTATTTTACCGCGGAAGTGA |
| | | GCCATTGCCGCGCGACCGAATATATTATGAAAGGCGTGTATATT |
| | | AACACCGCGCTGCTGAACGCGAGCTGCGCGGCGATGGATGATT |
| | | TTCAGCTGATTCCGATGATTAGCAAATGCCGCACCAAAGAAGG |
| | | CCGCCGCAAAACCAACCTGTATGGCTTTATTATTAAAGGCCGCA |
| | | GCCATCTGCGCAACGATACCGATGTGGTGAACTTTGTGAGCAT |
| | | GGAATTTAGCCTGACCGATCCGCGCCTGGAACCGCATAAATGG |
| | | GAAAAATATTGCGTGCTGGAAATTGGCGATATGCTGCTGCGCA |
| | | CCGCGATTGGCCAGGTGAGCCGCCCGATGTTTCTGTATGTGCGC |
| | | ACCAACGGCACCAGCAAAATTAAAATGAAATGGGGCATGGAA |
| | | ATGCGCCGCTGCCTGCTGCAGAGCCTGCAGCAGATTGAAAGCA |
| | | TGATTGAAGCGGAAAGCAGCGTGAAAGAAAAAGATATGACCA |
| | | AAGAATTTTTTGAAAACAAAAGCGAAACCTGGCCGATTGGCGA |
| | | AAGCCCGCGCGGCGTGGAAGAAGGCAGCATTGGCAAAGTGTGC |
| | | CGCACCCTGCTGGCGAAAAGCGTGTTTAACAGCCTGTATGCGA |
| | | GCCCGCAGCTGGAAGGCTTTAGCGCGGAAAGCCGCAAACTGCT |
| | | GCTGATTGTGCAGGCGCTGCGCGATAACCTGGAACCGGGCACC |
| | | TTTGATCTGGGCGGCCTGTATGAAGCGATTGAAGAATGCCTGAT |
| | | TAACGATCCGTGGGTGCTGCTGAACGCGAGCTGGTTTAACAGC |
| | | TTTCTGACCCATGCGCTGAAATAGATCG |
| 13 | IVT_NP | TAATACGACTCACTATAGGGATAATACCATGCGCTTTAACAGCC |
| | | AGCATCAGAGCGATAAACTGAGCAAAAGCCGCGTGGATAACCA |
| | | TAGCATGAGCGATATTGAAGCGATGGCGAGCCAGGGCACCAAA |
| | | CGCAGCTATGAACAGATGGAAACCGGCGGCGAACGCCAGGAT |
| | | GCGACCGAAATTCGCGCGAGCGTGGGCCGCATGATTGGCGGCA |
| | | TTGGCCGCTTTTATATTCAGATGTGCACCGAACTGAAACTGAGC |
| | | GATTATGATGGCCGCCTGATTCAGAACAGCATTACCATTGAAC |
| | | GCATGGTGCTGAGCGCGTTTGATGAACGCCGCAACAAATATCT |
| | | GGAAGAACATCCGAGCGCGGGCAAAGATCCGAAAAAAACCGG |
| | | CGGCCCGATTTATCGCCGCGTGGATGGCAAATGGATGCGCGAA |
| | | CTGATTCTGTATGATAAAGAAGAAATTCGCCGCGTGTGGCGCC |
| | | AGGCGAACAACGGCGAAGATGCGACCGCGGGCCTGACCCATAT |
| | | TATGATTTGGCATAGCAACCTGAACGATGCGACCTATCAGCGC |
| | | ACCCGCGCGCTGGTGCGCACCGGCATGGATCCGCGCATGTGCA |
| | | GCCTGATGCAGGGCAGCACCCTGCCGCGCCGCAGCGGCGCGGC |
| | | GGGCGCGGCGGTGAAAGGCGTGGGCACCATTGCGATGGAACTG |
| | | ATTCGCATGATTAAACGCGGCATTAACGATCGCAACTTTTGGCG |
| | | CGGCGAAAACGGCCGCCGCACCCGCGTGGCGTATGAACGCATG |
| | | TGCAACATTCTGAAAGGCAAATTTCAGACCGCGGCGCAGCGCG |
| | | CGATGATGGATCAGGTGCGCGAAAGCCGCAACCCGGGCAACGC |
| | | GGAAATTGAAGATCTGATTTTTCTGGCGCGCAGCGCGCTGATTC |
| | | TGCGCGGCAGCGTGGCGCATAAAAGCTGCCTGCCGGCGTGCGT |
| | | GTATGGCCTGGCGGTGGCGAGCGGCCATGATTTTGAACGCGAA |
| | | GGCTATAGCCTGGTGGGCATTGATCCGTTTAAACTGCTGCAGAA |
| | | CAGCCAGGTGGTGAGCCTGATGCGCCCGAACGAAAACCCGGCG |
| | | CATAAAAGCCAGCTGGTGTGGATGGCGTGCCATAGCGCGGCGT |
| | | TTGAAGATCTGCGCGTGAGCAGCTTTATTCGCGGCAAAAAAGT |
| | | GATTCCGCGCGGCAAACTGAGCACCCGCGGCGTGCAGATTGCG |
| | | AGCAACGAAAACGTGGAAACCATGAACAGCAACACCCTGGAA |
| | | CTGCGCAGCCGCTATTGGGCGATTCGCACCCGCAGCGGCGGCA |
| | | ACACCAACCAGCAGAAAGCGAGCGCGGGCCAGATTAGCGTGC |
| | | AGCCGACCTTTAGCGTGCAGCGCAACCTGCCGTTTGAACGCGC |
| | | GACCGTGATGGCGGCGTTTAGCGGCAACAACGAAGGCCGCACC |
| | | AGCGATATGCGCACCGAAGTGATTCGCATGATGGAAAGCGCGA |
| | | AACCGGAAGATCTGAGCTTTCAGGGCCGCGGCGTGTTTGAACT |
| | | GAGCGATGAAAAAGCGACCAACCCGATTGTGCCGAGCTTTGAT |
| | | ATGAGCAACGAAGGCAGCTATTTTTTTGGCGATAACGCGGAAG |
| | | AATATGATAGCTAGATCG |
| 14 | T7 promoter | TAATACGACTCACTATAGGGATAAT |
| 15 | miRNA-vRNA-5p | CTTGTTGATGCCTTAGCAGCACGTAAATATTGGCGTTAAGATTC |
| | | TAAAATTATCTCCAGTATTAACTGTGCTGCTGAAGTAGAAACAA |
| | | G |
| 16 | miRNA-vRNA-3p | CCTGCTTTTGCTTAGCTTATCAGACTGATGTTGACTGTTGAATCT |
| | | CATGGCAACAGCAGTCGATGGGCTGAGTAAAACATGG |

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1. Primary miRNA (Pri-miRNA) Design

This Example demonstrates the generation of synthetic primary miRNAs (pri-miRNAs) which, when processed, generate the shared 5′ end (i.e., miRNA-vRNA-5p) or the shared 3′ end (i.e., miRNA-vRNA-3p) of vRNA found within the influenza A virus (IAV) genera.

The success of the provided RNA derived system relies on the capacity of cells to recognize and process primary miRNA transcripts (pri-miRNAs) within the host cell. As microRNA biology is ubiquitous in multicellular organisms, the machinery required for metabolizing the RNA intermediates involved are also conserved. Moreover, as the recognition of pri-miRNAs is structural in nature, the sequence can be modulated and still enable efficient processing.

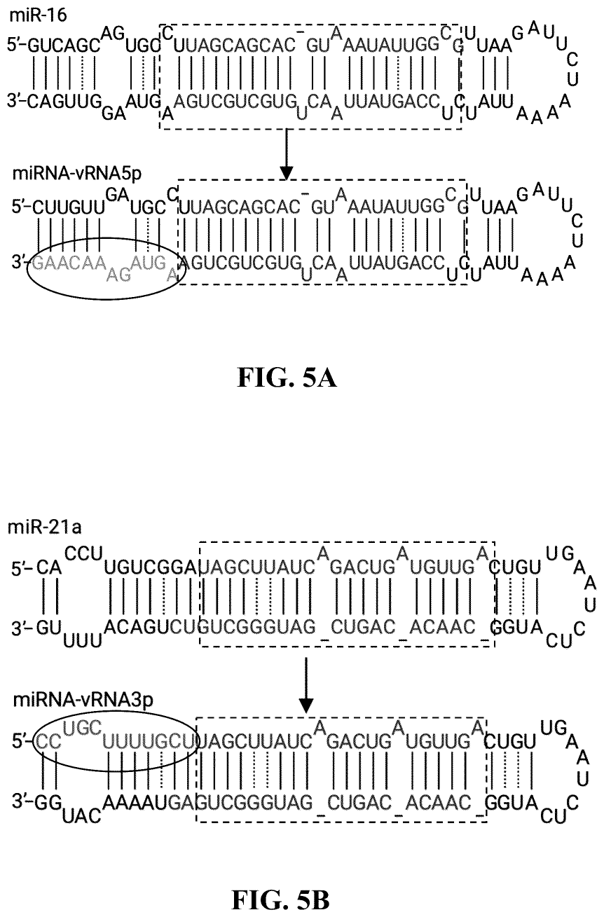

In this Example, sequences of the pri-miR-16 and pri-miR-21a hairpins were synthesized to enable the generation of distinct terminal ends in mammalian cells using an RNA that can be generated in the context of an in vitro transcription (IVT) reaction. These exemplary pri-miRNAs were chosen for their high level of processing efficiency as determined by RNA-Seq read captures on miRase (www.mirbase.org), and does not denote any other special attribute. Therefore, the selected pri-miRNAs are merely exemplary, and alternate pri-miRNA designs may be used in the provided systems. Modeling the predicted structure of hsa-miR-16, a synthetic primary miRNA was created that generates the shared 5′end of vRNA of IAV when processed, herein termed miRNA-vRNA-5p (FIG. 5A). To generate the 3′ vRNA terminal end, a similar strategy was applied to miR-21a, herein termed miRNA-vRNA-3p (FIG. 5B). As these hairpins are sufficient for host miRNA machinery recognition, adding genetic material to the 5′ or 3′ ends does not impact the generation of vRNA with the proper termini, but rather provides a more physiologically relevant primary transcript typically generated by the host. Given this, promoters and/or motifs (e.g., T7, SP6, T3, etc.) to ensure high fidelity in vitro transcription may be added as needed, as the terminal ends will be processed only following cytoplasmic entry to liberate the desired vRNA ends (circled portion of FIGS. 5A-5B).

### Example 2. Generation of vRNA Segments and RNA-Based Rescue of Influenza

This Example demonstrates the use of in vitro transcription (IVT) constructs generated from the pri-miRNA described in Example 1 to generate viruses or virus-derived vectors, independent of plasmid transfection.

### In Vitro Transcription

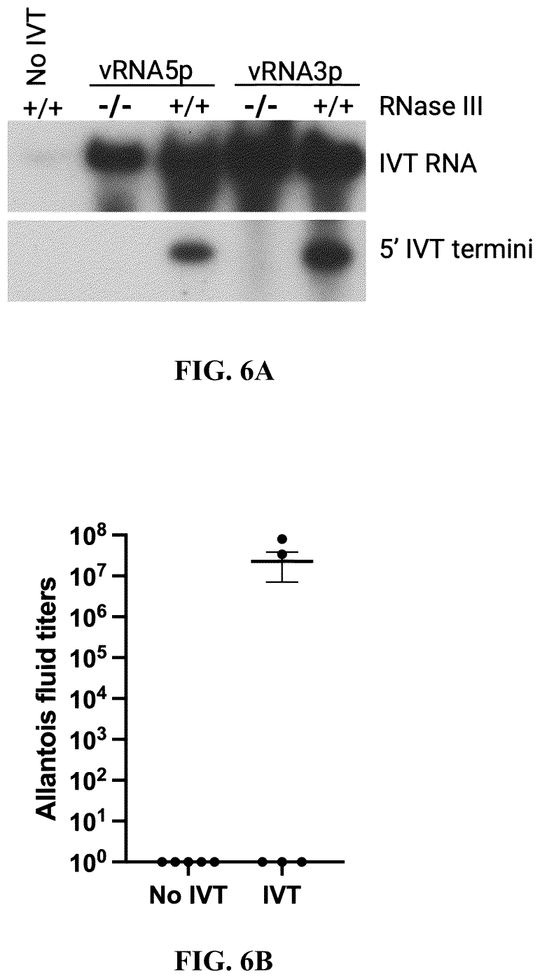

To determine whether proper processing could be achieved, IVT constructs of pri-miRNAs described in Example 1 were generated (FIGS. 5A-5B) by synthesizing a dsDNA fragment (i.e., IDT gBlock or synthetic gene product from Twist Biosciences) encoding these pri-miRNA flanked by a T7 promoter and terminator sequence. The RNA resulting from the in vitro transcription reaction were subsequently purified and end labelled with αP32 using T4 PNK as per the manufacturer's instructions (NEB®). Labelled material was then transfected into wild type 293 cells (RNaseIII^{+/+}) or cells lacking both Dicer and Drosha (RNaseIII^{-/-}), two essential components required for miRNA processing as described elsewhere (Aguado et al nature 547(7661) 114-117 (2017)). Total RNA was harvested 6 hours post transfection, resolved by SDS-PAGE, transferred to nitrocellulose, and exposed to film (FIG. 6A). These data demonstrate successful miRNA processing for both miR-vRNA-5p and miR-vRNA-3p.

Next, 8 gene fragments representing each of the 8 vRNAs were designed and synthesized to exploit this host processing activity and achieve a desired RNA sequence with defined terminal ends (gBlocks®, Integrated DNA Technologies). To generate these self-processing vRNA precursors, each gBlock included a 5′ end T7 full promoter (SEQ ID NO: 14). Downstream of the T7 promoter the miR-vRNA-5p hairpin was incorporated, followed by the vRNA sequence of interest in a 5′ to 3′ orientation, the miR-vRNA-3p motif, and finally any desired end sequence thereafter (SEQ ID NOs: 2-9). Similarly, codon optimized mRNA encoding PB2, PB1, PA, and NP were also synthesized in this way, but did not include the miR-vRNA motifs (SEQ ID NOs: 10-13).

To generate IAV, a total of 60 ng of gBlock template (5 ng/vRNA_5 ng/mRNA for PB2, PB1, PA, and NP) were used as input for the IVT reaction. IVT reactions were performed using HiScribe T7 RNA synthesis kit as per the manufacturer's instruction (NEB® E2040S). Briefly, we performed each reaction in 50 μL using 25U T7 RNA polymerase and 10 mM each of GTP, ATP, CTP, and UTP. After 3 hours at 33° C., RNA was purified using RNAClean XP beads (Beckman Coulter®), and residual template DNA was digested using the TURBO DNA-free™ (Invitrogen®), as per the manufacturer's instructions. Purified RNA subsequently underwent enzyme-based 5′ capping and A-tailing using commercial kits (NEB® #M2080 and #M0276, respectively). The pre-vRNA templates were capped to enhance their stability during the transfection process, however, this step is optional.

### RNA-Based Generation of IAV

Following purification of the IVT RNA, ˜25 μg of RNA was resuspended in rNase-free phosphate buffered saline (PBS) in a volume of 25 μL (Invitrogen®). Suspended RNA was then mixed with 25 μL Lipofectamine 3000 (Invitrogen®), also resuspended in rNase-free PBS in a volume of 25 μL. The mixture was subsequently incubated for 30 minutes at room temperature, and then used to inoculate ethanol-washed and candled, pathogen-free embryonated chicken eggs at day 10 post gestation. The injection site was plugged thereafter with wax and the eggs were incubated at 37° C. for 2 days prior to moving to 4° C. for 16 hours. Eggs were then re-washed in ethanol under sterol conditions in a laminar flow hood, candled, and the shell and membrane were removed to gain access to the allantois fluid. Using a pipette and spatula to distant the deceased embryo, ˜8 mL of fluid was removed and cleared of debris by centrifugation (1250 RPM, 5 minutes).

Standard plaque assay was used thereafter to assess virus rescue and titers, demonstrating successful generation of IAV with titers ranging from 10⁷-10⁸ plaque forming units per mL (FIG. 6B), and verified by sequencing.

Taken together, these data demonstrate that capacity of the host to recognize and process primary miRNAs can be exploited to enable the generation of IVT products that subsequently can be directly used to generate viruses or virus-derived vectors independent of plasmid transfection.

### Example 3. IAV Comprising a Heterologous Nucleic Acid Sequence DATA

This example demonstrates the generation of an influenza A virus (IAV) comprising a heterologous nucleic acid sequence encoding a green fluorescent protein (GFP).

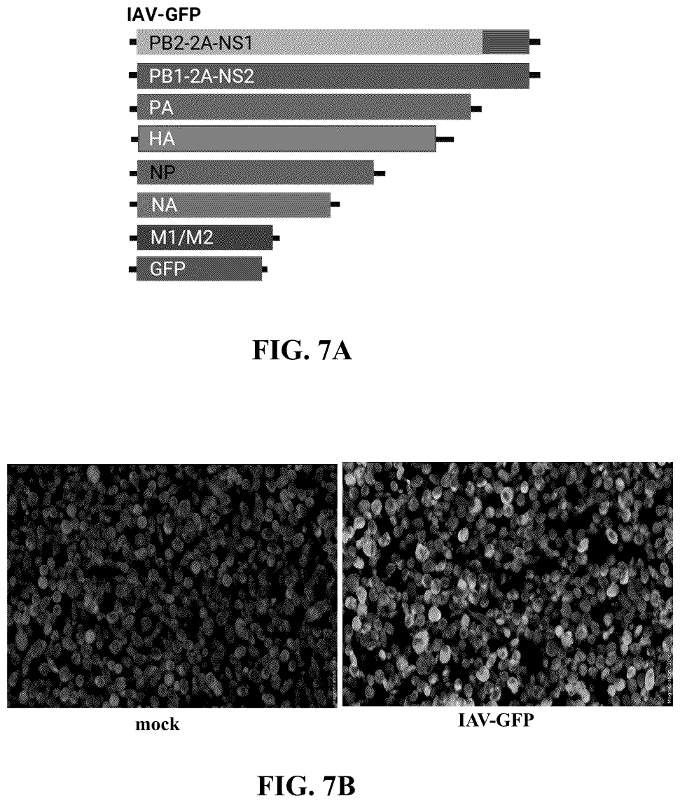

IAV expressing GFP (IAV-GFP) was generated by combining the IVT-vRNAs transcribed by the T7 promoter, using the method described in Example 2. The open reading frames (ORFs) for NS1 and NS2 were encoded as poly-cistronic RNAs using a 2A motif at the C-terminal end of PB2 and PB1 on segments 1 and 2 of the vRNA segmented genome of IAV, respectively. Segment 8 comprised the GFP ORF flanked by the non-coding RNA and packaging material of endogenous segment 8 of IAV (FIG. 7A). RNA encoding these 3 recombinant IVT-vRNAs, alongside the 5 wild type IVT-vRNAs, and IVT-mRNA for PB2, PB1, PA, and NP, were transfected into eggs to generate a replication competent IAV capable of expressing GFP as cargo (e.g., as a heterologous nucleic acid sequence on IVT-vRNA 8).

To demonstrate the expression of GFP, allantois fluid was added to primary human lung fibroblasts (Lonza). The cells were imaged 24 hours post treatment by RNA in situ hybridization for rRNA, GFP, and DNA (FIG. 7B). These data show that the RNA-based systems provided herein can successfully express protein from a heterologous nucleic acid sequence.

1. An RNA-based system for generating a virus of the Orthomyxoviridae family, or a recombinant derivative thereof, the system comprising:
(i) one or more mRNAs encoding the cognate viral RNA-dependent RNA polymerase (RdRp) and a nucleoprotein (NP); and,
(ii) a plurality of in vitro transcribed (“IVT”) viral RNA (“IVT-vRNA”) collectively comprising the genome of the virus,
wherein each IVT-vRNA of the plurality of IVT-vRNAs comprises a genomic segment of the virus (vRNA) and a miRNA hairpin structure located at the 5′ end of the vRNA, and
wherein the miRNA hairpin structure in the IVT-vRNA can be cleaved in a host cell comprising a miRNA processing machinery to produce the vRNA with the termini that are recognizable by the cognate viral RdRp for transcription and replication.

2. The RNA-based system of claim 1, wherein at least one of the IVT-vRNAs further comprises a miRNA hairpin structure at the 3′ end of the vRNA, wherein the miRNA hairpin structure at the 3′ end of the vRNA (i) is the same as or different from the miRNA hairpin structure at the 5′ end of the vRNA, and (ii) can be cleaved in a host cell comprising a miRNA processing machinery to produce the vRNA with the termini that are recognizable by the cognate viral RdRp for transcription and replication.

3. The RNA-based system of claim 2, wherein the at least one IVT-vRNA further comprises a poly A sequence at the 3′ end of the miRNA hairpin structure located at the 3′ end of vRNA.

4. The RNA-based system of claim 1, wherein the at least one IVT-vRNA further comprises a 5′ cap at the 5′ end of the miRNA hairpin structure located at the 5′ end of vRNA.

5. The RNA-based system of claim 1, wherein the miRNA hairpin structure has a stem length of about 25 to about 45 nucleotides.

6. The RNA-based system of claim 1, wherein the miRNA hairpin structure has an apical loop size of about 3 to about 23 nucleotides.

7. The RNA-based system of claim 1, wherein the miRNA hairpin structure comprises a CNNC nucleotide motif or an UG nucleotide motif.

8. The RNA-based system of claim 1, wherein the miRNA hairpin structure is derived from a primary miRNA (pri-miRNA).

9. The RNA-based system of claim 8, wherein the miRNA hairpin structure is derived from pri-miR-16 or pri-miR-21a.

10. The RNA-based system of claim 1, wherein the one or more mRNAs comprise a 5′ cap.

11. The RNA-based system of claim 1, wherein the one or more mRNAs comprise a 3′ polyA sequence.

12. The RNA-based system of claim 1, wherein the one or more mRNAs are on separate nucleic acid molecules.

13. The RNA-based system of claim 1, wherein the one or more mRNAs are provided in a single nucleic acid molecule.

14. The RNA-based system of claim 1, wherein cleavage of the miRNA hairpin structure(s) in the plurality of IVT-vRNAs in a host cell comprising a miRNA processing machinery produces transcriptionally active vRNAs.

15. The RNA-based system of claim 1, wherein the virus is replication competent or conditionally competent.

16. The RNA-based system of claim 1, wherein the virus is of a genus selected from the group consisting of Alphainfluenzavirus, Betainfluenzavirus, Gammainfluenzavirus, Deltainfluenzavirus, Isavirus, Thogotovirus, and Quaranjavirus.

17. The RNA-based system of claim 1, wherein the virus is an influenza virus.

18. The RNA-based system of claim 17, wherein the influenza virus is a Type A influenza virus (IAV), a Type B influenza virus (IBV), a Type C influenza virus (ICV), a Type D influenza virus (IDV), or a variant, subtype, or reassortant thereof.

19. The RNA-based system of claim 17, wherein the one or more mRNAs encode PB2, PB1, PA, and NP.

20. The RNA-based system of claim 17, wherein the plurality of IVT-vRNAs comprise:
(i) IVT-vRNA 1 for PB2;
(ii) IVT-vRNA 2 for PB1;
(iii) IVT-vRNA 3 for PA;
(iv) IVT-vRNA 4 for HA;
(v) IVT-vRNA 5 for NP;
(vi) IVT-vRNA 6 for NA;
(vii) IVT-vRNA 7 for M1 and M2; and
(viii) IVT-vRNA 8 for NS1 and NS2.

21. The RNA-based system of claim 1, wherein one or more of the IVT-vRNAs comprise a heterologous nucleic acid sequence.

22. The RNA-based system of claim 21, wherein the heterologous nucleic acid sequence is inserted at a 5′ non-coding region of the vRNA.

23. The RNA-based system of claim 21, wherein the heterologous nucleic acid sequence is inserted at a 3′ non-coding region of the vRNA.

24. The RNA-based system of claim 21, wherein the heterologous nucleic acid sequence comprises a complement sequence of a coding sequence encoding a recombinant protein.

25. The RNA-based system of claim 21, wherein the heterologous nucleic acid sequence has a length of about 800 to about 4,000 nucleotides.

26. A method of producing a plurality of viruses of the Orthomyxoviridae family, or recombinant derivatives thereof, in a cell culture, comprising:
(i) introducing the RNA-based system of claim 1 into a population of host cells, wherein the population of host cells is capable of supporting replication of the virus; and
(ii) culturing the population of host cells; and
(iii) recovering a plurality of the viruses.

27-30. (canceled)

31. A method of treating or preventing a disease or disorder associated with an infection by a virus of the Orthomyxoviridae family in a subject in need thereof, comprising administering to the subject an effective amount of the virus produced using the method of claim 26.

32-33. (canceled)

34. A DNA-based template for producing at least one of the plurality of IVT-vRNAs in the RNA-based system of claim 1, comprising a complementary DNA sequence corresponding to the at least one IVT-vRNA.

35-37. (canceled)

38. A method for producing at least one of the plurality of IVT-vRNAs in the RNA-based system of claim 1, comprising subjecting a DNA-based template to in vitro transcription, wherein the DNA-based template comprises a complementary DNA sequence corresponding to the at least one IVT-vRNA, thereby obtaining the at least one IVT-vRNA.
